SEQUENCE LISTING (20 sequences)

SEQ ID NO: 1 - DNA, 1425 bp, Artificial Sequence
  Synthetic construct
atgccaatagaaacaaaacgtcaggctgaagtgcttaaaaagctacaagatgtgataaaacatacagatcgtgacattgcggctggaagaaagttagctattaaaaggtgggtcgagacctatatagagtatatcaaactttttaaggatgataagctggaattcttatataatgtttttcgagatgaaggttgttggttaggtacaaggttaaataatactgttttaggtcagaaattgactgaagagaaaataggagaaatcgataacccactaccaaggtatggtatggcatctaggtactgtataacgggcaagataggtgattttttcaacaaacagtttgtactctctagaggtcaatttacttcagaagaggtagatagtcaaggtaatccgatcagtgatcaatatgtaagaaacattctgctatcatccatgaagagaaatggtcctgtgtttgatttctggatcgatagagaatctggggaattaaagaagtatgatgcagtagaaggttttgacagtactgtaaaacttaagtggagcgaaggggtagagtatttttataatcagttagaggaaaaagataaggagaagaagcttacagaagctattgttgctctttctcgtcctcaatctgttaagagagacgctcctattttagatttttgtgtaaggaatataggcgataaagatactcttttacagaaattattgcagaaagataagggagtatatttccttcttgctgaattaatagagtcatgtttttttgatacggttcatgatttggtacagtgctggtgttataaaggcgtttcagcaggaggagactgttcggacaagatattctcacagcaagactatgaactttttctttattcactttcaaatgtgatgttgaaaaatcctgagttaagtgttcaagctagatcccttattatggagatttggaaatgtgaacgctttgctgaatacagagagacctctgttaatacttctaattatacagttcctataaagagtgtacttgggggattaatcattaattggaaacgagaagatgtttgtaagcccgatagggaaatagagaaagaagaaatattagatatgatttcatttgccaaaggttgctttcctgaaaagtttgacctttttaaagaagtcatgatagaaaatcttagaatatgtggtagggaaggaaagaggaaaggtgtagattacggcaagtttgcagaagagttatttcttcagttagagaaagtaactttaccttctgtaggtgatggtccttggaataatttgcggtctcaatctaaggtatctttgccacttgatggttctggtgatggcccacagtctgagtttgaagctcctagtgtgagtggtatttctggttctcataagaaaagaagaatctag

SEQ ID NO: 2 - protein, 474 aa, Artificial Sequence
  Synthetic construct
MPIETKRQAEVLKKLQDVIKHTDRDIAAGRKLAIKRWVETYIEYIKLFKDDKLEFLYNVFRDEGCWLGTRLNNTVLGQKLTEEKIGEIDNPLPRYGMASRYCITGKIGDFFNKQFVLSRGQFTSEEVDSQGNPISDQYVRNILLSSMKRNGPVFDFWIDRESGELKKYDAVEGFDSTVKLKWSEGVEYFYNQLEEKDKEKKLTEAIVALSRPQSVKRDAPILDFCVRNIGDKDTLLQKLLQKDKGVYFLLAELIESCFFDTVHDLVQCWCYKGVSAGGDCSDKIFSQQDYELFLYSLSNVMLKNPELSVQARSLIMEIWKCERFAEYRETSVNTSNYTVPIKSVLGGLIINWKREDVCKPDREIEKEEILDMISFAKGCFPEKFDLFKEVMIENLRICGREGKRKGVDYGKFAEELFLQLEKVTLPSVGDGPWNNLRSQSKVSLPLDGSGDGPQSEFEAPSVSGISGSHKKRRI

SEQ ID NO: 3 - DNA, 3501 bp, Artificial Sequence
  Synthetic construct
gtggatggagatcttgatggttttagacaagagtttgaatcctttttagatcaatgtccatttttcttgtatcatgtaagtacaggacgtttccttcctgtattctttttcagtatgtttgctactgctcatgatgctaatatcttaaaagcaaatgagagagtgtattttcgttttgataatcatggtattgatacaggtggtagaaatagaaatacagggaacctaaaagtcgctgtttatcatgacggacagcaagttgtcagatgctacagtatttctgatcgtcttaatagtgatgggttaaggttcagtacaagggaaagaaatgctctagtgcgagagattagagggcaaaatccaaatttaagggaagaagacctaaattttgagcaatacaaagtatgcatgcatggaaagggcaagagtcagggagaggcgattgcaacagtattcgaggtgattcgtgaaaaagattctcaaggtagagatagatttgctaaatattcagcgtctgagattagccttcttaggcatatagaacgcaataggcttaatgggattaatgcgcctgcgccacgcagtttgttgacagttaaggaaataggaagtatacgactcaatcaagatcagagagtacagcttggtcatttggtcaattttgtgcaagttgcaccgggtcagcaagggattttcagttttatggaagtgctagcaagtaaccaaaaaataaatatagaacgtggaataaatgaaggaattttgccatacataactcgaatctatcgtagttacctaggcagcctacaaaatgacattcaaaatcgcagtcaaaagtttgagagtcacggatttttcttaggtttgttggcaaattttattcatctctacacaatagatattgaccttgacttgtctcctggaaattcatatgttgcttttcttatatgtcatcaggcagagagagaaaacattcctatcgttattaatgttactagatggaggacatcgtctgatattgcattaaaccgcgctagagctgatgctaaaagattacatgtttcttcatttatatctattcacactgaatcaagaaatgctgtttgtattggattaaattttaatctgaatatagatccttttagtattgatacagtagagtttttagagaatagatttcctttggtacaaagattatttgagtgtttggaggatgaaggaattagagaaaatattagagatttcttgcttcaacatcttcctaacgaaataccaagaaatgcagagaattataacagaatatttgattgcataactggttttgcttttgggaatagtattttagaagagttcagattagtaaacgcagttcaacaacgtgtaagaaagtatatatttagatatggtgatgagaatcatgctttaaccatggtcttccatactcaaggttctgatatagttatacttcatattagagataacaacgctgtacaacaaggagccatcaatttacaagatcttaatgttgacggaaataatgttcatgtacgggaagtttcatgcacacttaataatcaacttggccttaatattcatacagataaccttggtttatatcacaattaccaaaataataatgcaaataattttcttggtggtaatcttgtgcaagtgcctaatgctggaaatgtgcataatgctttaaatcaagttatgaatgatggctggcaagatagatttcagcatcaagaattatttagaaacatttctgcagtattaatgccagaagatacgcatggcaatatgataatagatgtaaatagcaaagataagtttcgctctatactacatggtacattttatgctagtgataatccttataaagtgcttgctatgtataaagttggtcaaacatatagtttaaaaaggtggcaggaagaagaaggagaaagggtaatacttacaagagttacagaacagagactaggtcttctattattaagacaacctacagcagatactcacccaattggatatgtattaggatttgctgataatgcagaagaagtagaacaggagcaagacgaggcaaggtacaaaataacagaattgatgagcaaacaaaggggatatttgcctattacttctggaaatgaggtggttttgtcttatgctgtatttaatagaggtgcacagagagcagaagactttatatctcttccacaacaagcagtgtatgtacatagacttgatcgtcgtggtcatgactcaagaccagaagtattagtgggacctgaaagtgttattgatgaaaatccaccagaaaatctattgtcagatcaaactcgtgaaaatttcaggcgcttttacatggaaaaaagaccaggacagaactcgatttttttgcttgatatagatgataatctgcacgttccatttagttacttgcaaggtactagagcacaggcaatagaaacattaaggtcaagaataaggggaggtggtacttctacagcacaaggaatattacaacaaataaacactatccttcgtagaaacaacgctcgtgaaatagaagatgtgcataatctacttgcactagactttgcaacagaaaatcaaaatttccgttattggctacaaactcatgacatgtttttcgctgcacgacaatatactttccatgatgatcgatctaatccaactaatgatcgtcatgattttgcaataacttcagtaggagtcgatggaaatcaaaatgatccaacaggtagggacttattaagtagtaacatagataactttaaacaaaaagtagattcgggtgaaaaagatagattaactgctattattaatgtaggtaatcgtcattgggttacattagttattgtccaccaaaatggaaattattatgggtattatgctgattcacttggtccagatagtcgtattgacaataatattcgaggagctttaagagaatgtgatattagcgatgataatgtccatgatgtttccgttcatcagcaaacagatggccataattgtggcatatgggcatacgaaaatgctagggatattaaccaagctattgatcaagctttacagggaaatagtaactttggagagaaaggtgaaggtattataggttatatacgtggtcttcttagtgcaggaattggaaatgacactagacaacctcaaagaaatgaacaatactttagaaatcggagaagaaatatttcacaattattccaaaatgattctctatcttctcctaggggtagattgattcaaggtcgtccaggaattcaacatgaaattgatccattactattacaatttttagaactccaatatccacagcgtggaggtgggggagcattgcaattaggcggagaaagagtgatatcaattgattttggtccgcaatctgtattggatgaaattgatggagtgaatagagtttatgatcatagcaatggtagaggcagtaggtag

SEQ ID NO: 4 - protein, 1166 aa, Artificial Sequence
  Synthetic construct
MDGDLDGFRQEFESFLDQCPFFLYHVSTGRFLPVFFFSMFATAHDANILKANERVYFRFDNHGIDTGGRNRNTGNLKVAVYHDGQQVVRCYSISDRLNSDGLRFSTRERNALVREIRGQNPNLREEDLNFEQYKVCMHGKGKSQGEAIATVFEVIREKDSQGRDRFAKYSASEISLLRHIERNRLNGINAPAPRSLLTVKEIGSIRLNQDQRVQLGHLVNFVQVAPGQQGIFSFMEVLASNQKINIERGINEGILPYITRIYRSYLGSLQNDIQNRSQKFESHGFFLGLLANFIHLYTIDIDLDLSPGNSYVAFLICHQAERENIPIVINVTRWRTSSDIALNRARADAKRLHVSSFISIHTESRNAVCIGLNFNLNIDPFSIDTVEFLENRFPLVQRLFECLEDEGIRENIRDFLLQHLPNEIPRNAENYNRIFDCITGFAFGNSILEEFRLVNAVQQRVRKYIFRYGDENHALTMVFHTQGSDIVILHIRDNNAVQQGAINLQDLNVDGNNVHVREVSCTLNNQLGLNIHTDNLGLYHNYQNNNANNFLGGNLVQVPNAGNVHNALNQVMNDGWQDRFQHQELFRNISAVLMPEDTHGNMIIDVNSKDKFRSILHGTFYASDNPYKVLAMYKVGQTYSLKRWQEEEGERVILTRVTEQRLGLLLLRQPTADTHPIGYVLGFADNAEEVEQEQDEARYKITELMSKQRGYLPITSGNEVVLSYAVFNRGAQRAEDFISLPQQAVYVHRLDRRGHDSRPEVLVGPESVIDENPPENLLSDQTRENFRRFYMEKRPGQNSIFLLDIDDNLHVPFSYLQGTRAQAIETLRSRIRGGGTSTAQGILQQINTILRRNNAREIEDVHNLLALDFATENQNFRYWLQTHDMFFAARQYTFHDDRSNPTNDRHDFAITSVGVDGNQNDPTGRDLLSSNIDNFKQKVDSGEKDRLTAIINVGNRHWVTLVIVHQNGNYYGYYADSLGPDSRIDNNIRGALRECDISDDNVHDVSVHQQTDGHNCGIWAYENARDINQAIDQALQGNSNFGEKGEGIIGYIRGLLSAGIGNDTRQPQRNEQYFRNRRRNISQLFQNDSLSSPRGRLIQGRPGIQHEIDPLLLQFLELQYPQRGGGGALQLGGERVISIDFGPQSVLDEIDGVNRVYDHSNGRGSR

SEQ ID NO: 5 - DNA, 1476 bp, Artificial Sequence
  Synthetic construct
atgccaacacagaaagagcttcgggatacgatgtccaaaaaattacaggaagctattaaacatccagatccagcagttgttgccgggaggaagtcagctatcaagagatgggtgggagtccttcaagataactttatggagcacataaaatactttaagggtgataagttgaagtttttgcacaatgtatttcaagatgaaggttgctggtcaggtgtaaggttggataatgctgctttaggtcaaaggtttactgaagaaaaaataggtggaatagataatccacttcgcaaatatgagatggcttgtagttactgtgtggtggataaaattcatcctctctttcaaaaaagatttgaatcttataggaacaagtttcctcctggtgcatttgatggtaaaactgaaactgaatttggcaaatacgtacgaaactcgttactagatagcataaagaggaaaggtcctgtatttgatttctggattgatagagaatctggggaattaaagaagtatgatgcagtagaaggttttgacagtgctgtaaaatttaagtggagtgaaggggtagagtatttttataatcatttaaaagaagaagataaggaaaagaagctcacagaagctattcttgctctttctcgcgttcaatctgttgagaaagacgcccctattttagatttttgtgtaaataagatagtcgataaagatactcttttacagaaattatcacagaaagataaaggagtatattccctttttgctgaattaatagagtcatgtttttttgatacggttcatgatttggtacagtgctggtgttataaagaagtttcagcaggaggagaccattcagagaaaatattctcacagcgagactatgagctttttctttcctctctttcagacacaatgttgaaaaatcctgagttaagcgttcaagctagatctcttattatggaattttgggaatgtggtagcttgtatcaatacagaaaagctgctgttaatacttctaattatacagttcctacaagtggtgtatttgcagagttaatagtcaattggagacgagaagacatttataagactgatgaagaaaaagaaatagagaaaaaagaaatattagatatgatgtcatttgccaaagattgctttcctgaaaagtttgagctctttaaaaaactaataataagagaccttagattatgcggtagggaaggtaaaagagtaaatgtagattacggtctgtttgcagaagaattattctctgagttagagaaaacaattttaccacctggtcctgtaggtgatggtccttgcagtaatttgcgatcacgatctaaagctcatggtagtaagaaaacaactttgccagttgatgatagtccgcagtctgagcttggaactcctagtgtaagtggtgtttcttcttataagaaaaaaagcgtctttacgcttagtggtaataagtaa

SEQ ID NO: 6 - protein, 491 aa, Artificial Sequence
  Synthetic construct
MPTQKELRDTMSKKLQEAIKHPDPAVVAGRKSAIKRWVGVLQDNFMEHIKYFKGDKLKFLHNVFQDEGCWSGVRLDNAALGQRFTEEKIGGIDNPLRKYEMACSYCVVDKIHPLFQKRFESYRNKFPPGAFDGKTETEFGKYVRNSLLDSIKRKGPVFDFWIDRESGELKKYDAVEGFDSAVKFKWSEGVEYFYNHLKEEDKEKKLTEAILALSRVQSVEKDAPILDFCVNKIVDKDTLLQKLSQKDKGVYSLFAELIESCFFDTVHDLVQCWCYKEVSAGGDHSEKIFSQRDYELFLSSLSDTMLKNPELSVQARSLIMEFWECGSLYQYRKAAVNTSNYTVPTSGVFAELIVNWRREDIYKTDEEKEIEKKEILDMMSFAKDCFPEKFELFKKLIIRDLRLCGREGKRVNVDYGLFAEELFSELEKTILPPGPVGDGPCSNLRSRSKAHGSKKTTLPVDDSPQSELGTPSVSGVSSYKKKSVFTLSGNK

SEQ ID NO: 7 - DNA, 3525 bp, Artificial Sequence
  Synthetic construct
atgagtaatggtgatggacttattaggagtttggtggatggagatcttgaaggattcagacaaggatttgaatcttttttagatcaatgtccatctttcttgtatcatgtaagtgcaggtcgtttccttcctgtattcttttttagtatgttttctactgcacatgatgctaatatcttaaatgcaaatgagagagtctattttcgttttgataaccatggtgttaatccacgtaatggtgaaaatcgaaatacggcaaacctaaaagttgctgtttatcgtgacggacagcaagttgtcagatgctacagtatttctgatcgtcctaatagtgatgggttgaggttcagtacaagggagagaaatgctctagtacaagagattagacggcaaaatccaaatttaagggaagaagacctaaattttgagcaatacaaagtatgcatgcacggaaagggcaagagtcagggagaggcaattgcaacggtattcgaggtaattcgtgaaaaagatcgtcaaggtagggataaatttgccaaatattcagcatctgaggttcatttcttgaggcaactctttagaaatcacagattaacaattaaggaaatagaaggaagacaactcaatcaaaatcagctcagacaacttggtaggtcagtcaattttacacgagtagaaccaggtcagcagaggattgacaactttatggaaatgctagcaagtaaccaaagacaagatgtaagggattctctccgaggagatattttagaatatgtaactgatacctataacaattatagggcacagatagaaaataatattgaaggtcgcagtcaaaagtttgagagtcatgggtttttattaggtttcttagcaaattttagtcatcgctacacaataggcgtcgatcttgacttatctcctagaaactcacatgttgcatttcttgtacgtcatcaagtagaaagagaaaatattcctattgttattaatcttgctacaagggcaccgccctatatcgcattaaaccgcgccagaagtcacgctgaaagattgcatgttttttcatttatacctatccatactgaatcaagaaatactgtctgtgttggattaaattttaatttaaatctagatccttttagtgttgatacagtagggcttcaacaggatagatttcctttagtacaaagattatttgagtgtttggagaatgaaggaattagagaaaatattagagatttcttgcttcaccatcttcctgctgaaataccaagaaatgcagagaattatgatagaatatttgattgcataactggttttgcttttgggaatagtgcttttgataggcaccctttagaactagaagaggaagacgaagcacctataacaaagtacatatttagacatggtgatgagggtttaagatgtttaactatggtctttcatgctgaaggttctgatatagttatacttcatattagagctcacgatgcgcaacaacaaggagccatcaatttacagactcttaatgttaatggaaatgatgttcatgtgtgggaagtttcatgcacacttaataatcaacttgaactagatattgatctaccaaatgaccttggtttatatcacgattaccaaaataataatgcaaataattttcttgctggtgatcttgtacaagtgcccaatactgaaaatgtacataatactttaaatcaagttgtgaatgatggctggaaaaatatagctcagcatagaggattatttcaagagatctctggagcattgatgccgcttgtggatacaataaatgttaatagtgaggataagttccgttctatactacatggtacattttatgctagtgataatccttataaagtgcttgctatgtataaagttggtcaaacatatagtttaaaaagggggcaggaagaagaaggagaaagggtaatactcacaagaattacagaacagagattagatcttttattattaagacaacctagagagaatgacctagatactcacccaattggatatgtgttaagacttgctaataatgcagaagaagtaggacaacagcaaaatgatgcgagacaagaaatcggaagacttaagaaacaacacagaggatttatacctattacttctggaaatgaggtggttttgtttcctattgtgtttaatagagatgcacacgaagcaggtaatcttatacttttcccagaagggataggaagagaagagcatgtacacaggcttgatcgtcatgttcgcagctcaagaccaggaggattagtgggacctgaaagtgttattgatgaaaatccaccagaaggtctattatcagatcagactcgtgaaaactttaggcgtttttacgaagaaaaagcaccaggacaaaattcgatttttttgcttgatataggcgacaatctacatgttccctttagttacttgcaaggtactagagcacaggtaatagaaacattaaagtcaagaataaggggaggtggtactcctacagcacaaggaatattacaacaaataaatgctatccttcgtagaaacaacgctcgtgagatagaagatgtgcatgatctacttgcactagactttgcaacagataatcaaaattatcgttattggctacaaactcatgacatgtttttcgctgcacgacaatatactttccttgataatcaatctcattcaactaatgatcattatggttttgaaataacttcagtaggagtcgatggaaatcaaaatgatccaacaggtaggggcttattaagtagtcacataactaactttaaacaaaaagtagattcgggtgaaaaagatagattaattgctattattaatgtaggtaatcgtcattgggttacattagttattgtacaccaaaatggaaattattatgggtattatgctgattcacttggtccagatagtggtattgacaataatattcgaggagctttaagagaatgtgatattaacgatgataatgtccataatatttccgttcatcagcaaacagatggccataattgtggcatatgggtatacgaaaatgctagggatattaaccaagctattgatcaagctttacagggaaataataactttggagagaaaggtgaaggtattataggttatatacgtggtcttcttagtgcaggcattggaaatgacactagacaacctcgaagaaatgaacaatactttgaagatcggagaagagatatttcacaattactccaaaatgatcctaacttaccttctcgccggagtgatttaattcaagctcatccaggaattcaacatgaaattgatccattactattacaatttttaggactccaatacccacagcgtggaggtggaggagcattacaattaggcggagaaagagtgatatcaattgattttggtaacccgcagtctgcattagataaaattgatggagtgagtagagtttataaccatagcaatagtagaggtagtaggtag

SEQ ID NO: 8 - protein, 1174 aa, Artificial Sequence
  Synthetic construct
MSNGDGLIRSLVDGDLEGFRQGFESFLDQCPSFLYHVSAGRFLPVFFFSMFSTAHDANILNANERVYFRFDNHGVNPRNGENRNTANLKVAVYRDGQQVVRCYSISDRPNSDGLRFSTRERNALVQEIRRQNPNLREEDLNFEQYKVCMHGKGKSQGEAIATVFEVIREKDRQGRDKFAKYSASEVHFLRQLFRNHRLTIKEIEGRQLNQNQLRQLGRSVNFTRVEPGQQRIDNFMEMLASNQRQDVRDSLRGDILEYVTDTYNNYRAQIENNIEGRSQKFESHGFLLGFLANFSHRYTIGVDLDLSPRNSHVAFLVRHQVERENIPIVINLATRAPPYIALNRARSHAERLHVFSFIPIHTESRNTVCVGLNFNLNLDPFSVDTVGLQQDRFPLVQRLFECLENEGIRENIRDFLLHHLPAEIPRNAENYDRIFDCITGFAFGNSAFDRHPLELEEEDEAPITKYIFRHGDEGLRCLTMVFHAEGSDIVILHIRAHDAQQQGAINLQTLNVNGNDVHVWEVSCTLNNQLELDIDLPNDLGLYHDYQNNNANNFLAGDLVQVPNTENVHNTLNQVVNDGWKNIAQHRGLFQEISGALMPLVDTINVNSEDKFRSILHGTFYASDNPYKVLAMYKVGQTYSLKRGQEEEGERVILTRITEQRLDLLLLRQPRENDLDTHPIGYVLRLANNAEEVGQQQNDARQEIGRLKKQHRGFIPITSGNEVVLFPIVFNRDAHEAGNLILFPEGIGREEHVHRLDRHVRSSRPGGLVGPESVIDENPPEGLLSDQTRENFRRFYEEKAPGQNSIFLLDIGDNLHVPFSYLQGTRAQVIETLKSRIRGGGTPTAQGILQQINAILRRNNAREIEDVHDLLALDFATDNQNYRYWLQTHDMFFAARQYTFLDNQSHSTNDHYGFEITSVGVDGNQNDPTGRGLLSSHITNFKQKVDSGEKDRLIAIINVGNRHWVTLVIVHQNGNYYGYYADSLGPDSGIDNNIRGALRECDINDDNVHNISVHQQTDGHNCGIWVYENARDINQAIDQALQGNNNFGEKGEGIIGYIRGLLSAGIGNDTRQPRRNEQYFEDRRRDISQLLQNDPNLPSRRSDLIQAHPGIQHEIDPLLLQFLGLQYPQRGGGGALQLGGERVISIDFGNPQSALDKIDGVSRVYNHSNSRGSR

SEQ ID NO: 9 - DNA, 1371 bp, Artificial Sequence
  Synthetic construct
atgccaaaaaagatggagcgtcatgctgcagtgcttagtaagttaaagagtgttattcaacatacagattccaaggtcatggctgaaaggcgttcagctattgaaagatgggtaaaaacttacattaggcaggtagaatatcttaaagatgataagctacaattcttatacaacatatttcgcgatgaaagttgttggtcaggtacgagattgaacaatacaatcttaggacagaggtttactgaagaaaaaataggcgaaataaagaaccctcttcctatatatgatatggcatgtcgatactgcgtgatagataaaattcctttgctctttcagaagcagtttgaatcttacaaaagtagcttctcttctgaagagatagatgatgatggtaagcctgcaactagcaataacaaatatgtaaagagtgagttgttgggttatatgaagagtcaagaccctgtatttagcttttgggttgataaaaaatctggagaatttaagaagcatgtcagcgcaacagaaggatttaagaaagctatagaacttaagtggagcgaaggagtagaatatttttatagccttctaaatgaaaaagaaagagaaagagaaaggaaaattactgatgcagttactatattatcctctgttcaatgtgaccataatggtgctgttactttagacttttgtcttagtaaaatgagcgatcaagcaaaaaacaagctgtttaaagattctgagctatcaaaaaaagataaaggagtgtactctctctttagcgcgttgatacatcaaggtttttttgatacgatgcaagctatacttccgatgtttaaagataaaatactggaggataagatactttcacctaggagttatactcttcttctctcctcactttcggacatgatgctcgaaaattctgagtcaactattcaagctagggaagctataatgaaccttataaagtgtggtaatttcaataatcatgaggggcgtgaggaaaaagctgcggtatttttttctaatggaagggttccgattaagcgtgcgcttgcaggattgattgtcgattggcaacttggttgtacaaaaaaggaagaggtgttaaaggtactacagtttgccaaagagttttgtgcagttgaaagttttatgtattttaaaaaatctgttgttgataacctaaaaatggttggtagggatggtatgagaaaaaatatagactatggtaaattagcagaaaagttgtttgctgaattagatacggtatccgtgcctaacggaagaggtgattttggtggagctggtgacccacagtctacactaggaagcactgaagttagtagtttttctggtcgcaataagtag

SEQ ID NO: 10 - protein, 456 aa, Artificial Sequence
  Synthetic construct
MPKKMERHAAVLSKLKSVIQHTDSKVMAERRSAIERWVKTYIRQVEYLKDDKLQFLYNIFRDESCWSGTRLNNTILGQRFTEEKIGEIKNPLPIYDMACRYCVIDKIPLLFQKQFESYKSSFSSEEIDDDGKPATSNNKYVKSELLGYMKSQDPVFSFWVDKKSGEFKKHVSATEGFKKAIELKWSEGVEYFYSLLNEKERERERKITDAVTILSSVQCDHNGAVTLDFCLSKMSDQAKNKLFKDSELSKKDKGVYSLFSALIHQGFFDTMQAILPMFKDKILEDKILSPRSYTLLLSSLSDMMLENSESTIQAREAIMNLIKCGNFNNHEGREEKAAVFFSNGRVPIKRALAGLIVDWQLGCTKKEEVLKVLQFAKEFCAVESFMYFKKSVVDNLKMVGRDGMRKNIDYGKLAEKLFAELDTVSVPNGRGDFGGAGDPQSTLGSTEVSSFSGRNK

SEQ ID NO: 11 - DNA, 2265 bp, Artificial Sequence
  Synthetic construct
atgcatgggttagttagaagtttaataaatggaaattgtggagaattcacggaaaagtttgaatatttcttggattcatgtccatcttttctgcattcagttggcaaagatcacttttttcctgcgttcttttttggcatgtttgctactgcacatgattctggtgttgcaaacaatgatgaaagaatcttctttcgttttgataatgatccaggtagtcctggaaggggaaatctaaaggttgcaattctaacaactgatggaaataacagaagagttgtaaggtgctatactattgctgacagagagaatagctacggttctaggtttagccagcaggaaagggagcagctggaaggtatcctgcgagatgaagagcttgaatggcaagagtataaaacatttatatgggcggataatcaaggtgaagatgaagaagaggaagcagtaagatgtaggatatttcaggcaggacaagggccgtttactggaaatcatgcatcttatttaactcgtagacatagttttcaagagattaccagaacacctgggctgcaaaataattatttaccggatttgatgaatcagctagaaagtgatgatgcagatgatgtacacgacactactgaggaagtgtttcagcatattattggtgtctacgatagatatagtcaggcattggacttctatggtagagagtctgactatcatggttttgtttccggtgttttgatgcattttagatatcgcaatgtagccaatatttaccttgagctgtttgtaggtggtggatatgcagatattacttctattgtacgtggtacacagaggttaattaattctgttccctgtgtaactgaacttaaggcaggcagaagagcagataggaatgctggccgtgcattagagcaggctggaaattatgttaatggatgtcccgtttcatccatatctattccaacattatcaccaagagctgtctccgctggagtgaatttcgattttggtaacccaggacgtttacagcttggtgtgagggcttttttagcaaaaggttcttctttaatggaaagattatttgaacctgtagaggatgaggagattggagaaaatgttagggattatctactccatccagcctttggtgtacctgctgtaccaggtattaggaataggggtggtgttaacgctagagatagaagaatatttctctatacaagtggatttgctttcgcaagtattgcatttgcaaaaggaactgtgccaatagaaggaaatcgtgcaatagtagataagcacttgtttcactatgacggtaatgcaaaaatgttagatgagcaaagatacaatacacaagtaaatattggagatcgtgctttgactatggttttgcatgtatcacgaggtagagaccagaaggaggaggtgatcgtatttcatgttcgccacgtattggctaatcaactttttccggacaatggattggatctatcgcgttggccgaatgctatggtacatgaagtggtgtgtaatttgaccataaatagaaggacaagaggagtaaatgataatcttggtttaactgttaatgtagaaacatttgactcgcctgctgactacctgcttgatagaggtaatcagccttttcaaggtgagcttttacgaataggtggcgttagtaatgtgcatcgcgctgcaaatgtaatgatgaatactggctgggaaaatgaagatccagacagtcatgaacggttttaccaagcaatttccaacgtgctaaatccaccccagccaaataatgcaggactccaatcattagcatgggtagtgaacagagataatgctagagaagctgggtttcatgctgcattgcatggattattttacacttgcgataatcctgctagggtagttagtgaatttcaggttggaggaggaggaaagttagacttagtattgtcacgagctataggaaggatgggaggtacttatccgattggaacagagctaaagtttgctgccactgaagcagacgtacaaaatagagaagaagaagcagatgaacaggtggagggttatctgcagagtagagggtttgatcgcattactgatggagataaaatggttttctcgtatgccgtatttaatgatcaagcgccagcaccagcacaaaatgtcccaaatacccttatagcagttagtaatgttctacgcataaaagataacttaggaattgacactgtggacgactttccttatagataa

SEQ ID NO: 12 - protein, 754 aa, Artificial Sequence
  Synthetic construct
MHGLVRSLINGNCGEFTEKFEYFLDSCPSFLHSVGKDHFFPAFFFGMFATAHDSGVANNDERIFFRFDNDPGSPGRGNLKVAILTTDGNNRRVVRCYTIADRENSYGSRFSQQEREQLEGILRDEELEWQEYKTFIWADNQGEDEEEEAVRCRIFQAGQGPFTGNHASYLTRRHSFQEITRTPGLQNNYLPDLMNQLESDDADDVHDTTEEVFQHIIGVYDRYSQALDFYGRESDYHGFVSGVLMHFRYRNVANIYLELFVGGGYADITSIVRGTQRLINSVPCVTELKAGRRADRNAGRALEQAGNYVNGCPVSSISIPTLSPRAVSAGVNFDFGNPGRLQLGVRAFLAKGSSLMERLFEPVEDEEIGENVRDYLLHPAFGVPAVPGIRNRGGVNARDRRIFLYTSGFAFASIAFAKGTVPIEGNRAIVDKHLFHYDGNAKMLDEQRYNTQVNIGDRALTMVLHVSRGRDQKEEVIVFHVRHVLANQLFPDNGLDLSRWPNAMVHEVVCNLTINRRTRGVNDNLGLTVNVETFDSPADYLLDRGNQPFQGELLRIGGVSNVHRAANVMMNTGWENEDPDSHERFYQAISNVLNPPQPNNAGLQSLAWVVNRDNAREAGFHAALHGLFYTCDNPARVVSEFQVGGGGKLDLVLSRAIGRMGGTYPIGTELKFAATEADVQNREEEADEQVEGYLQSRGFDRITDGDKMVFSYAVFNDQAPAPAQNVPNTLIAVSNVLRIKDNLGIDTVDDFPYR

SEQ ID NO: 13 - DNA, 1473 bp, Artificial Sequence
  Synthetic construct
atgccaaaaagtaaaactaaacgtggaacggaagatttgaagggtaatgcaggcccaagcaaaagatctcgtctcagttctgatcctaaaaaaaataaagagattatctctagcaaagtaataagtaagctgaaggatgttgttaaaggtgatagaacttcagctattgaggaatgggtcaaggctcaccctgtcacagtagagggtctaatcgttgagcaatcggacctcttatgtaatgcgtttcgtgatgaatcttgttggtcaggtgcgacactagatgttgctaaattggtaggagaattagctaaatcaggtgtgttgaatccatttgctatatataaaatagcatgtattgagtgtgtagagagtgaaattaagcaattatttgacaaggcgttagagtcttttagatctgacttatctcataaaggtgcatgtgaggaagataggaatttagcttgcagtgataagcttgcaagagttgaattgttaagttccatgggaagacgtgatcctgttttcaatttctggattgatcaagaatcaggtaaccttagagaaaatatagaagcagaagatggatttaataaggctgtagatttcaagtggagtaagggagtggaacacttctataatcgtctgtgttctgaagaaaaattagtgaaagaagagagagaaaaattgctagtttctgctattgcaaaattatctccattgcaatctagctataaacttgcttctaccttaaattcccttctaggtaaagtcataagcgcaaaagtagatcataagtcactacttgggctaccgaataagagagataggggtgtgatctatcgtcctcttagttacttagtagagcacggttttctttgcacaactaagtatgttatccagtacttgagcgagggatgttcaagatctgaagtagagaaaatgctttcacctagaggatatgcacatcttctctcatcgctttcatttgttgtagtttctaaagattatgacttggataacaggaatgaagcaaggtcagctattagcagtctttgggaatctagtgtatttaaccaaaataaaataaatgttgtcgatccttttaaagataggattgcttttgttgcaatggaaaatgcaatttcaaatttgattgtagatcaggagaacagtaaggatactcaaagtgctggcgatggtgaaaaagttgatttggtcttgagtattttaaagtttgctaaagattgttgttcagacaaaagctttaaatcattaaaagcgaggatagcaaatagtttagataaaacaaggaattctaagatgatagatgcaactagctcctgcaatttaatagaagagttgtgtaagtcagcgagaaatttgaatttattctctgctagcactgaaggtcctcaatctacgttagtgggtactaatgttagtatttcgcctgctgcagttgttaacaaatag

SEQ ID NO: 14 - protein, 490 aa, Artificial Sequence
  Synthetic construct
MPKSKTKRGTEDLKGNAGPSKRSRLSSDPKKNKEIISSKVISKLKDVVKGDRTSAIEEWVKAHPVTVEGLIVEQSDLLCNAFRDESCWSGATLDVAKLVGELAKSGVLNPFAIYKIACIECVESEIKQLFDKALESFRSDLSHKGACEEDRNLACSDKLARVELLSSMGRRDPVFNFWIDQESGNLRENIEAEDGFNKAVDFKWSKGVEHFYNRLCSEEKLVKEEREKLLVSAIAKLSPLQSSYKLASTLNSLLGKVISAKVDHKSLLGLPNKRDRGVIYRPLSYLVEHGFLCTTKYVIQYLSEGCSRSEVEKMLSPRGYAHLLSSLSFVVVSKDYDLDNRNEARSAISSLWESSVFNQNKINVVDPFKDRIAFVAMENAISNLIVDQENSKDTQSAGDGEKVDLVLSILKFAKDCCSDKSFKSLKARIANSLDKTRNSKMIDATSSCNLIEELCKSARNLNLFSASTEGPQSTLVGTNVSISPAAVVNK

SEQ ID NO: 15 - DNA, 2091 bp, Artificial Sequence
  Synthetic construct
atgcatggtaataatgaagatcgtgaattagttagggctttattaagtggaggttgtgatgagtttagtagacaatttgtaggttttttaaacaactgtccatcttttttgcattcggctaataagcctggcttttttcctacattcttttttggtatgttttctactgcacatgatgcaggtatattagttgaaggtgaaagagtctattttcgttttgacaattatggaaatctaaaagttgctgttctcactaataaagaaaatagaagaatagtcaggtgttatactgttgctgataatgagaacagccctgggtcaaggtttagtgcagaagagaagcagcaggtagaagagaatcttccacaagaattacaggaagatgaggatctggattgggaagagtataaaatatttcggtttggagaagaatgtaggtttattcatgaaatagatagatttcctcaacgtgatgaacctggagctccaatttttcatgaaattaacccaatcagagaacaaggtgaattgttagacctgatgagtgagttggcaaatgacgatacaggagaagtgcgtactaatgttaaaagaattttggaatatgttattgatatccatgatgaacatgaagatagcttagtgtttcgtgcagagtctgactaccacggttttctgtgtgggtttttagtaaattttagataccgagctttggctgatttctacccagagctacttataggaaaaggttatgcagatgttgttttgcttgttcgtggtgttgatcagacaaatgattcggttccaattataattgagttgaaggttggtgatgaggaaggattagagcaagctaaagattatgctaaaagttgttctgtttcgtctttgcctattcatacctcatcaccaagtgctgtttgtgtagcgttaaattttcaattacgtggaggtgctggtctccgaacttctgtgcaggccttttcagaaggtggtctttccttaataccgggtttactacatcctcatggaaatggagttaggggaaatgtaaaacgttttttacaacccatagcatcagagttcactcaatcgcctcattgtaacactttttcctgtacttcatcgtttgtttttggaaatgttttatctacaaggagggacttagaaacaaatgatgggcgggaggtaagggttaccaagtatctatttaaccactctcagggagagaaaatgaaacgtacaggtggtagaggagatgcagcagatattgtaagccatgcgttaactttagctctatttttatcaaatattggttttgttgtgcttcacatttttcgtcgtttaaagtggcagactttaccagacaaggcattgaacctgtcgttactgcctcaagccacagatgatgctaaggtgcgtcaagtactttgtgaagtagatgtccagggtcatctggaagtggcttctgcaaagaaattcgaatcactacgtgcttactcacgttctcatagtgaaggttatttcgagggaaggttttcagaacaaatgggtaatgttaggaatttacatcaacttgcagatcagttgatgagtgctgagcctaattttggtaatgatggtaatgttaatggtgagtacagggctaggtatgaagttttatttaatgagatttctcgtctgttgtctccgttattaaatggaaaccgtctactcgtgaacaatgaagctaaatttcaggctttgttgcgtggaatatttcaaaattgcgataatcctgccaaggtaattattgagttccagctacagagaggaaggaaaatagacctagtattatcaaaatctgcggaaaatgatgatactcatccaattggaatagagttgaagtatgctaacaccgcagaacaagttgaacgaaaaagggtggaggcaaatcgacagttaagtgaatacgaattttgtggaggatgcaagcgtattactgggggagatgcgatggttttgttatacgctatattaaatgctgtaggacaagagcaggatctgatattgattggtgggcttcgtagagcatctgggttttctagatga

SEQ ID NO: 16 - protein, 696 aa, Artificial Sequence
  Synthetic construct
MHGNNEDRELVRALLSGGCDEFSRQFVGFLNNCPSFLHSANKPGFFPTFFFGMFSTAHDAGILVEGERVYFRFDNYGNLKVAVLTNKENRRIVRCYTVADNENSPGSRFSAEEKQQVEENLPQELQEDEDLDWEEYKIFRFGEECRFIHEIDRFPQRDEPGAPIFHEINPIREQGELLDLMSELANDDTGEVRTNVKRILEYVIDIHDEHEDSLVFRAESDYHGFLCGFLVNFRYRALADFYPELLIGKGYADVVLLVRGVDQTNDSVPIIIELKVGDEEGLEQAKDYAKSCSVSSLPIHTSSPSAVCVALNFQLRGGAGLRTSVQAFSEGGLSLIPGLLHPHGNGVRGNVKRFLQPIASEFTQSPHCNTFSCTSSFVFGNVLSTRRDLETNDGREVRVTKYLFNHSQGEKMKRTGGRGDAADIVSHALTLALFLSNIGFVVLHIFRRLKWQTLPDKALNLSLLPQATDDAKVRQVLCEVDVQGHLEVASAKKFESLRAYSRSHSEGYFEGRFSEQMGNVRNLHQLADQLMSAEPNFGNDGNVNGEYRARYEVLFNEISRLLSPLLNGNRLLVNNEAKFQALLRGIFQNCDNPAKVIIEFQLQRGRKIDLVLSKSAENDDTHPIGIELKYANTAEQVERKRVEANRQLSEYEFCGGCKRITGGDAMVLLYAILNAVGQEQDLILIGGLRRASGFSR

SEQ ID NO: 17 - DNA, 1338 bp, Artificial Sequence
  Synthetic construct
atggaatctggtttggatcacaattacaataaaatacttgatatattaaaaggtgctattaaaggcgacgataatcaagttaaagcaagaaaacaccttagagtagaaagatggttgagggcttatattcaattaattgaagattttgatgaggaaaaactaatttttttttctgatatattctctgataattcttgttgggatggaataaaattaaagaataaagctgttggtgaaaggctaactgaagaaaaaaataaaaatggaaaagaaaatccgcttgatcttgcagatagatattacttggcatgtaaatattgtctagaagataagattcctggattatttgaacaagtatttatgagatttaagagaagtgcctttgaagaagatggatctgatgatgatctgagaagagaattattggaaaatatcgaagaaactagccctatagaagctttctggtcttttcttattgataagcagattggaaaactaaacgaatataaatcagttgaaggtttgcaaaaatccatacagataaattctaataaaaactgggaagaaggtatagagttcttctataataaattacacaatgattccagtatttctagtcaagataaagatgatctgttaattgaagcagctttatctgcagtaaagggttacaaagaagtagacaccatagagttttgcctgtctaaaatggatgatgagcaaaagaaaaaattactagatagagattataaggaaaatacttattatgcagtgttgaatgtgctagtaggtcagtattactttgattcttttatggaattaagccgattgtgtagtcagattgaatgtgaacgttacacaacttttttatcttcattatcagatcaagtactgaagaatccagatctgtctgaagaaacaaaaaaatgtatgatgaatgtttgggaacgtataataaaattaaaaactcaagaccgcggggagcaatctatttcctctatttttgtagactattcagttacatatacaatagcaaatttaattgtggatccaagtagacaaggggtaagtaaagaagaaatattagggaagatattaaagcacgtaaaagaaatgagtggtgaagagatgataaaggttaaagattctgtattaagtaaaattcagttatttcatgggggtaaaaaattgcagttaggagaacaagtattttctaaattagctcaagaagcttctaaagaatcaattttgcgtgaagctggtgatactttgccacagtcaagtctcagtacgactgataccccatataatataaaatctttaagccatagcaaatag

SEQ ID NO: 18 - protein, 445 aa, Artificial Sequence
  Synthetic construct
MESGLDHNYNKILDILKGAIKGDDNQVKARKHLRVERWLRAYIQLIEDFDEEKLIFFSDIFSDNSCWDGIKLKNKAVGERLTEEKNKNGKENPLDLADRYYLACKYCLEDKIPGLFEQVFMRFKRSAFEEDGSDDDLRRELLENIEETSPIEAFWSFLIDKQIGKLNEYKSVEGLQKSIQINSNKNWEEGIEFFYNKLHNDSSISSQDKDDLLIEAALSAVKGYKEVDTIEFCLSKMDDEQKKKLLDRDYKENTYYAVLNVLVGQYYFDSFMELSRLCSQIECERYTTFLSSLSDQVLKNPDLSEETKKCMMNVWERIIKLKTQDRGEQSISSIFVDYSVTYTIANLIVDPSRQGVSKEEILGKILKHVKEMSGEEMIKVKDSVLSKIQLFHGGKKLQLGEQVFSKLAQEASKESILREAGDTLPQSSLSTTDTPYNIKSLSHSK

SEQ ID NO: 19 - DNA, 2199 bp, Artificial Sequence
  Synthetic construct
atgccaagtaatgtcaagccgcttgagttggtacagcttctgttaatgagaaataaatcaaaagacgagttcctagattttcaaaaaaggttccaatcgtttatcaatcaatctccttcttttttgcattcagttggaaagccaggctttttccctagtttcttttttggtatgtttgctactgtattagacacagaacttgctactaaaattggtattaaaaaacttcattttcgttttgatgataatagaactttaaaaatagctatattaactaatgagggacttaagtgtataacgatgtctgatcaagttgatggtaacatgcatctaaagttctctcaaggagagttagaaaaaatagcacagaaatggaaaatgggagcagagtttgataaactagaaaaagaagagcatgaaataacaattacaggaaaagaagtaaagcacggaaaggttgatccagcttttagtaaaaagactgattattcacaaaaaggttttacagaaatagaaaaagatcgtgaccaacaagacctagagagcttaatttcaaaattgagtaatcaagatttcgaagaagtaaaaaagaacgctagaagaatgtttaattatattacaaatgtctataagaaatatgaaaaagaaactctatttagcggtaaagaatcaagtcatcatgggtttttagctgggtttttgataaattttaagtatcgttttcacctaaaactttatctcgaattatttgctggaaaaggttacgcagacattattttgcttgtgcgcggttctgataagtcgctaagctctattcctattattattgagcttaaagcaggtactggtgagataagtacagtgataaaagcattgaagcaagcacaagattatgttaagggctctttttctaactctataagaatgattactatagctaatgaagctatttgtgtaggattaaattttgacatggttcatcacgaaaatgttaaaattgatgtagaaaattttcttagtcgagaaggtaattctgtaatagaaaagttacttggcactgaagcaacgaatgctgaggtgataagaacacagctagagtatctttactatggaattgtttggagcaatggtggaagtgataatattaattatgtcagcagaatgatcttaggtcagctagtacttatttctaatattattaagcgtgaaaagttaggtaaacatatttttatttatgatcaaaatgataaaatggttactggatcacagaaacgcccagaagcagcaaaagaaagtattgaggattgtgttacaactatagtgctaactttaggtaagaaggtgcttatactcaacataaatgaaaaaaatgaatttgcattgagagtgccagataataaaggaattcctattgaaaatattaggagaattcaaaacgtcaatgacataaagatacaagaaataacctgtaacttatacagtacgcctagtaataagaatccatttgatcagtactgtaataagaataagggaattacagtaaatacgtatgactcattggacaaatacaaaagaggtaaagaaattttacaaggtaattttactcgaattgtggaaaataaaaaatttaaagcagctttgagcaaagctatagaatctggtaaatatgatgattacaaaaaactatttgaagaaatttctcatatactacatcctttcaaatcattaataagcaatgaggctacatttcaagctgtattgcatggtttatttagtagctacggagaagataatataaaagttattactgaatttcaaataggtggtggagagaagttggatgttatgttggttataaatgctactgatcaaaaaaaagaataccccccagttggaatagagctaaaatttgctaagaaaggagaattggataaaaaagaaaaagatgctaaggaccagttgaaaagatataaagaaggtgaagcgtataaggtaattactgatgctggcaaagtgaaactgatatatgctgtttttaataaaggtgcaacagatgaaggttcccttataaaaattggtaatgagtttgtagaggtagatgtaagacatagctctgtggttgcttttggtcaacagccaggtagtctccaacaaccttatgttaaacaagcaggtctatctcgagcagttaatcagtga

SEQ ID NO: 20 - protein, 732 aa, Artificial Sequence
  Synthetic construct
MPSNVKPLELVQLLLMRNKSKDEFLDFQKRFQSFINQSPSFLHSVGKPGFFPSFFFGMFATVLDTELATKIGIKKLHFRFDDNRTLKIAILTNEGLKCITMSDQVDGNMHLKFSQGELEKIAQKWKMGAEFDKLEKEEHEITITGKEVKHGKVDPAFSKKTDYSQKGFTEIEKDRDQQDLESLISKLSNQDFEEVKKNARRMFNYITNVYKKYEKETLFSGKESSHHGFLAGFLINFKYRFHLKLYLELFAGKGYADIILLVRGSDKSLSSIPIIIELKAGTGEISTVIKALKQAQDYVKGSFSNSIRMITIANEAICVGLNFDMVHHENVKIDVENFLSREGNSVIEKLLGTEATNAEVIRTQLEYLYYGIVWSNGGSDNINYVSRMILGQLVLISNIIKREKLGKHIFIYDQNDKMVTGSQKRPEAAKESIEDCVTTIVLTLGKKVLILNINEKNEFALRVPDNKGIPIENIRRIQNVNDIKIQEITCNLYSTPSNKNPFDQYCNKNKGITVNTYDSLDKYKRGKEILQGNFTRIVENKKFKAALSKAIESGKYDDYKKLFEEISHILHPFKSLISNEATFQAVLHGLFSSYGEDNIKVITEFQIGGGEKLDVMLVINATDQKKEYPPVGIELKFAKKGELDKKEKDAKDQLKRYKEGEAYKVITDAGKVKLIYAVFNKGATDEGSLIKIGNEFVEVDVRHSSVVAFGQQPGSLQQPYVKQAGLSRAVNQ

(19) United States
(12) Patent Application Publication
(10) Pub. No.: US 2021/0112792 A1
(43) Pub. Date: Apr. 22, 2021

(54) METHODS OF CYTOPLASMIC INCOMPATIBILITY-BASED TRANSGENICS FOR PEST OR VECTOR CONTROL

(71) Applicant: Vanderbilt University, Nashville, TN (US)

(72) Inventors: Seth R. BORDENSTEIN, Nashville, TN (US); John Dylan SHROPSHIRE, Nashville, TN (US)

(21) Appl. No.: 17/040,623

(22) PCT Filed: Apr. 10, 2019
(86) PCT No.: PCT/US2019/026736

§ 371 Date: Sep. 23, 2020

Related U.S. Application Data
(60) Provisional application No. 62/655,389, filed on Apr. 10, 2018.

(51) Int. Cl. A01K 67/033; C07K 14/195; C12N 15/87

(52) U.S. Cl. CPC A01K 67/0337; C12N 15/87; C07K 14/195

(57) ABSTRACT

The disclosure relates to improved methods of cyto-plasmic incompatibility-based transgenics for pest or vector control. Further disclosed are improved gene drivers for use in genetically modified arthropods and use in methods for controlling and/or reducing arthropod populations.

### CROSS REFERENCE TO RELATED APPLICATIONS

This application claims the benefit of U.S. Provisional Patent Application Ser. No. 62/655,389 filed Apr. 10, 2018, which is expressly incorporated herein by reference.

### STATEMENT REGARDING FEDERALLY SPONSORED RESEARCH

This invention was made with government support under Grant Nos. R21HD086833 and R01AI132581 awarded by the National Institutes of Health. The Government has certain rights in the invention.

### FIELD

The disclosure relates to improved methods of cytoplasmic incompatibility-based transgenics for pest or vector control. Further disclosed are improved gene drivers for use in genetically modified arthropods and use in methods for controlling and/or reducing arthropod populations.

### BACKGROUND

Wolbachia are an archetype of maternally-inherited, intracellular bacteria. They occur in an estimated 40-52% of arthropod species and 47% of the Onchocercidae family of filarial nematodes, making them the most widespread bacterial symbiont in the animal kingdom. In arthropods, Wolbachia mainly reside in the cells of the reproductive tissues, transmit transovarially, and often commandeer host fertility, sex ratios, and sex determination to enhance their maternal transmission via male-killing, feminization, parthenogenesis, or cytoplasmic incompatibility (CI).

Discovered nearly half a century ago, Wolbachia-induced CI is the most common reproductive modification and results in embryonic lethality when an infected male mates with an uninfected female, but this lethality is rescued when the female is likewise infected. As such, rescue provides a strong fitness advantage to infected females, the transmitting sex of Wolbachia. Alone, CI-induced lethality is deployed in vector control studies to crash the resident uninfected mosquito population through release of Wolbachia-infected males. Together, CI-induced lethality and rescue constitute a microbial drive system that is used in field studies worldwide to stably replace an uninfected mosquito population with an infected one via release of male and females harboring wMel Wolbachia, which confer resistance against dengue and Zika viruses. The efficacy of this drive system for spreading Wolbachia in target populations critically depends on Wolbachia's ability to rescue its own lethal modification of the sperm.

While CI is gaining momentum as a natural, sustainable, and inexpensive tool for vector control, the genes that underpin this microbial adaptation are not fully known. A previous screen of Wolbachia genomes and transcriptomes from infected ovaries identified two adjacent genes, cifA and cifB, from the wMel strain in Drosophila melanogaster as the only genes consistently associated with CI. These two genes occur in the eukaryotic association module of prophage WO, and they together recapitulate CI when dually expressed in uninfected male flies. Each gene alone is incapable of inducing CI, and the rescue gene remains unknown. What is needed are improved expression systems to test rescue, improved drive systems for induction of CI, and improved systems for use in vector control.

The systems and methods disclosed herein address these and other needs.

### SUMMARY

Disclosed herein are improved genetically modified bacteria, genetically modified bacteriophage, and genetically modified arthropods useful for controlling and/or reducing populations of arthropods (for example, insects). As cifA and cifB are the only two wMel genes associated with cytoplasmic incompatibility (CI), it was previously unknown whether the CI induction and rescue genes might be the same. In addition, previous gene drivers did not produce complete CI induction. Here, the inventors have shown that transgenic expression of the cifA gene using the nos-Gal4:VP16 gene driver (or the maternal triple driver (MTD)) from wMel Wolbachia in ovaries was surprisingly found to fully rescue CI and nullify associated embryonic defects. Thus, disclosed herein are improved gene drivers for use in microbial drive systems for vector control.

In some aspects, disclosed herein is a genetically modified arthropod, said arthropod comprising:
at least one bacterial gene encoding a cytoplasmic incompatibility factor or a variant thereof;
a promoter operably linked to the at least one bacterial gene, wherein the promoter comprises a Gal4 binding site; and
a nos-Gal4:VP16 gene driver;
wherein the expression of the cytoplasmic incompatibility factor in a male arthropod causes a reduction in viable offspring in comparison to a male arthropod lacking the cytoplasmic incompatibility factor.

In some embodiments, the genetically modified arthropod further comprises an additional gene driver. In some embodiments, the additional gene driver is a nos-GAL4-tubulin gene driver. In some embodiments, the additional gene driver is an otu-Gal4:VP16 gene driver. In some embodiments, the genetically modified arthropod further comprises a nos-GAL4-tubulin gene driver and an otu-Gal4:VP16 gene driver. In some embodiments, the genetically modified arthropod comprises the maternal triple driver (MTD-GAL4).

In some embodiments, the at least one bacterial gene is from Wolbachia. In some embodiments, the at least one bacterial gene is from wMel.

In some embodiments, the at least one bacterial gene encodes the cytoplasmic incompatibility factor CifA (WD0631). In some embodiments, the at least one bacterial gene encodes the cytoplasmic incompatibility factor CifB (WD0632). In some embodiments, the at least one bacterial gene encodes the cytoplasmic incompatibility factors CifA (WD0631) and CifB (WD0632).

In some embodiments, the at least one bacterial gene is from Wolbachia pipientis. In some embodiments, the at least one bacterial gene encodes the cytoplasmic incompatibility factor CidA^{wPip} (wPa_0282). In some embodiments, the at least one bacterial gene encodes the cytoplasmic incompatibility factor CidB^{wPip} (wPa_0283). In some embodiments, the at least one bacterial gene encodes the cytoplasmic incompatibility factors CidA^{wPip} (wPa_0282) and CidB^{wPip} (wPa_0283). In some embodiments, the at least one bacterial gene encodes the cytoplasmic incompatibility factor CinA^{wPip} (wPa_0294). In some embodiments, the at least one bacterial gene encodes the cytoplasmic incompatibility factor CinB^{wPip} (wPa_0295). In some embodiments, the at least one bacterial gene encodes the cytoplasmic incompatibility factors CinA^{wPip} (wPa_0294) and CinB^{wPip} (wPa_0295).

In some embodiments, the reduction in viable offspring is greater than 50%. In some embodiments, the arthropod is an insect. In some embodiments, the insect is selected from the mosquito genera consisting of Aedes, Culex and Anopheles. In some embodiments, the insect is selected from the group consisting of Aedes albopictus, Aedes aegypti and Aedes polynesiensis. In some embodiments, the insect is Drosophila suzukii.

In some aspects, disclosed herein is a method for controlling a population of target arthropods, comprising:

providing at least one bacterial gene encoding a cytoplasmic incompatibility factor or a variant thereof, and a promoter operably linked to the at least one bacterial gene, wherein the promoter comprises a Gal4 binding site;
transforming a population of male arthropods with the at least one bacterial gene, wherein the male arthropods comprise a nos-Gal4:VP16 gene driver; and
releasing the male arthropods amongst a population of target arthropods, wherein the release of the male arthropods reduces the population of target arthropods.

In some aspects, disclosed herein is method for controlling a population of target arthropods, comprising:
providing a genetically modified bacterium comprising:
   at least one bacterial gene encoding a cytoplasmic incompatibility factor or a variant thereof, and
   a promoter operably linked to the at least one bacterial gene, wherein the promoter comprises a Gal4 binding site;
infecting a population of replacement arthropods with the genetically modified bacterium, wherein the replacement arthropods comprise a nos-Gal4:VP16 gene driver; and
releasing the replacement arthropods amongst a population of target arthropods, wherein the release of the replacement arthropods reduces the population of target arthropods.

In some aspects, disclosed herein is method for controlling a population of target arthropods, comprising:
providing a genetically modified bacteriophage comprising:
   at least one bacterial gene encoding a cytoplasmic incompatibility factor or a variant thereof, and
   a promoter operably linked to the at least one bacterial gene, wherein the promoter comprises a Gal4 binding site;
infecting a population of replacement arthropods with the genetically modified bacteriophage, wherein the replacement arthropods comprise a nos-Gal4:VP16 gene driver; and
releasing the replacement arthropods amongst a population of target arthropods, wherein the release of the replacement arthropods reduces the population of target arthropods. in some embodiments, the bacteriophage comprises bacteriophage WO of Wolbachia.

### BRIEF DESCRIPTION OF THE DRAWINGS

The accompanying figures, which are incorporated in and constitute a part of this specification, illustrate several aspects described below.

FIG. 1. cifA rescues cytoplasmic incompatibility when it is highly expressed throughout oogenesis. (A) Hatch rate assays were conducted with transgenic expression of cifA under the control of nos-GAL4-tubulin (germline stem cells) or MTD-GAL4 (oogenesis) drivers. Each dot represents a replicate. Rescue occurred only under MTD-GAL4 expression. Horizontal dotted lines from top to bottom separate cross types with CI, cifA expression, and rescue. Wolbachia infections are represented by filled sex symbols and expressed genes are noted to the right of the corresponding sex. n=27-59 for each experimental cross across two experiments (both shown). Vertical bars represent medians, and letters to the right indicate significant differences based on α=0.05 calculated by Kruskal-Wallis and Dunn's test for multiple comparisons. (B) Expression fold change of cifA relative to the Drosophila housekeeping gene Rp49 was determined on a subset of abdomens from female expressing cifA via MTD-GAL4 or nos-GAL4-tubulin with 2^{-ΔΔCt}. Horizontal bars represent medians with 95% confidence intervals, and letters above indicate significance based on a Mann-Whitney test. In both cases, statistical comparisons are between all groups. Hatch rate experiments testing expression of cifA under MTD-GAL4 or nos-GAL4-tubulin have been repeated four and five times respectively.

FIG. 2. Rescue of cytoplasmic incompatibility is specific to cifA. Hatch rate assays were conducted with transgenic expression of cifA, cifB, and cifA; B using the MTD-GAL4 driver for expression throughout oogenesis. Each dot represents a replicate. Wolbachia infections are represented by filled sex symbols and expressed genes are noted to the right of the corresponding sex. n=11-29 for each experimental cross. Vertical bars represent medians, and letters to the right indicate significant differences based on α=0.05 calculated by Kruskal-Wallis and Dunn's test for multiple comparisons. Statistical comparisons are between all groups. Hatch rate experiments testing expression of cifA under MTD-GAL4 have been repeated three times.

FIG. 3. cifA rescues embryonic defects caused by cytoplasmic incompatibility. The number of embryos with each cytological phenotype resulting from the indicated crosses is shown. All replicate crosses were conducted in parallel and with sisters from the experiment in FIG. 2. cifA, cifB, and cifA; B transgene expression was under the control of MTD-GAL4. Wolbachia infections are represented by filled sex symbols and expressed genes are noted to the right of the corresponding sex. Letters to the right indicate significant differences based on α=0.05 calculated by pair-wise chi-square analyses comparing defects (all shades of red) against normal (blue) with Bonferroni adjusted p-values. This experiment has been conducted once.

FIG. 4. Ka/Ks sliding window analysis identifies cifA regions evolving under negative selection. A sliding window analysis of Ka/Ks ratios between cifA homologs from wMel and wHa rejects the neutral expectation of Ka/Ks=1 using a 25 amino acid sliding window across most of cifA. (a) Strong purifying selection is observed in several cifA regions including the sequence preceding the Catalase-rel domain Shaded regions denote previously described protein domain predictions (33).

FIG. 5. cifA transgene expression in germline stem cells fails to elicit rescue. Transgene expression of cifA, cifB, and cifA; B using the nos-GAL4-tubulin driver does not lead to rescue of cytoplasmic incompatibility. Each dot represents a replicate. Wolbachia infections are represented by filled sex symbols, and expressed genes are noted to the right of the corresponding sex. n=15-34 for each experimental cross. Vertical bars represent medians and letters to the right indicate significant differences based on α=0.05 calculated by Kruskal-Wallis and Dunn's test for multiple comparisons. Statistical comparisons are between all groups.

FIG. 6. cifA does not preferentially rescue one sex over the other. Surviving offspring from the experiment displayed in FIG. 2 were collected for adult sex ratio counts. There was no significant difference between any of the crosses. A sex ratio count was not possible for CI crosses due to the low number of surviving offspring. Wolbachia infections 430 are represented by filled sex symbols and expressed genes are noted to the right of the corresponding sex. n=11-22 for each experimental cross. Vertical bars represent medians and letters to the right indicate significant differences based on α=0.05 calculated by Kruskal-Wallis and Dunn's test for multiple comparisons. Statistical comparisons are between all groups.

FIG. 7. CifA is a putative cytoplasmic protein. (A) The PSORTb subcellular protein localization web server was used on Type I CifA proteins to predict the protein's localization in the Wolbachia cell. Predictive scores above 7.5 are accepted to be sufficient to determine a single location of localization and suggest that CifA is a cytoplasmic protein. (B) The TMpred web server was used to predict transmembrane helices. TMpred scores exceeding 500 (denoted by horizontal dotted line) are considered significant. TMpred scores were generated for transmembrane helices spanning from inside to-outside (i-o) and outside-to-inside (o-i). Shaded regions denote previously described protein domain predictions (33).

FIG. 8. cifA regions evolve under negative selection. (A) Pairwise codon-based z-tests of selection suggest that regions of the cifA gene are not evolving under the neutral expectation of Ka=Ks. Values below the diagonal are p-values for where there is a significant departure from neutrality or not. Values above the diagonal are the difference of Ka−Ks in which positive values suggest positive selection and negative values suggest purifying selection. (B) Pairwise Fisher's exact tests of neutrality suggest that cifA evolves under purifying selection. Values below the diagonal are p-values. If the p-value is less than 0.05, then the null hypothesis of strictly neutral or purifying selection is rejected. If the observed number of synonymous differences per synonymous site exceeds the number of nonsynonymous differences per nonsynonymous site then MEGA sets P=1 to indicate purifying selection, rather than positive selection. (C) SWAKK and JCoDA were used for sliding window analyses of Ka/Ks ratios between cifA homologs of wMel and the bidirectionally incompatible wHa. Both programs were performed with 25 (shown) and 454 50 amino acid (data not shown) windows and yield Ka/Ks ratios evident of strong purifying selection in the N-terminus region preceding the Catalase-rel domain and weaker purifying selection beyond it. Shaded regions denote previously described domain predictions (33).

FIG. 9. Fertility is related to strain genotype. A meta-analysis of all control rescue crosses (infected male x infected female) without a transgene shows that clutch size and hatch rate are significantly correlated for both the MTD-GAL4 and nos-GAL4-tubulin genotypes (r 460=0.59 and 0.50 for MTD-GAL4 and nos-GAL4-tubulin respectively), but the two strains have different y-intercepts (4.69 to 31.43 and 39.94 to 59.04 for MTD-GAL4 and nos-GAL4-tubulin respectively). Each dot represents a replicate where circles and diamonds are MTD-GAL4 (n=91) and nos-Gal4-tubulin (n=134) respectively. Vertical dotted lines represent embryo counts where 99% of clutch sizes with 0% embryo hatch rate are to the left for nos-GAL4-tubulin (left line) and MTD-GAL4 (right line). Correlation was assessed with Spearman Rho. A linear regression best-fit line is plotted for each genotype.

FIG. 10. Schematic of experimental methodology. (a) All experimental setups begin with the generation of the maternal lineage (pink), derived from GAL4 driver lines and collected as virgins and aged for 6-8 days till the peak of their fecundity. (b) The paternal lineage (blue) is setup in a stagger such that the males used in the experiment emerge on the day of the experiment. (c) Flies are crossed in a fashion dependent on the ultimate intent, and grape-juice agar plates provided and replaced in a similar manner for all experiments. Sex ratio studies are derived from hatch rate assays.

FIG. 11. Two-by-One model of CI is governed by cifA and cifB genes in the Eukaryotic Association Module of prophage WO in Wolbachia. The Two-by-One model of CI predicts that D. melanogaster males and females can be engineered to recapitulate both CI and rescue phenotypes in the absence of Wolbachia, thus depending on phage genes for successful reproduction. Schematics are not to scale. Insect, sperm, and embryo art were obtained and modified using vecteezy.com. Phage gene schematic modified from Lepage et al. 2017. CifA and CAB protein annotation from Lindsey et al. 2018. Purple indicates Eukaryotic Association Module genes as indicated by Bordenstein & Bordenstein 2016.

FIG. 12. cifA_{wMel} and cifB_{wMel} induce strong CI when transgenically expressed in males under the nos-GAL4:VP16 driver. (A) Two different driver lines, nos-GAL4-tubulin (top) and nos-GAL4:VP16 (bottom) were tested for their ability to induce CI when transgenically expressed in male Drosophila. Filled sex symbols represent infection with wMel Wolbachia and gene names to the right of a symbol represent expression of those genes in the corresponding sex of that cross. Vertical bars represent medians. Letters to the right indicate significant differences with an α=0.05. (B) To test if nos-GAL4-tubulin and nos-GAL4:VP16 generate different levels of gene expression, cifA_{wMel} fold expression difference relative to the Drosophila housekeeping gene rp49 in male abdomens under the two drivers was measured using qPCR. Males tested for gene expression were the same used in the hatch rate experiment in A.

FIG. 13. cifA_{wMel} can induce strong rescue when expressed in females under the nos-GAL4:VP16 driver. (A) Two different driver lines, nos-GAL4:VP16 (top) and otu-GAL4:VP16 (bottom), were tested for their ability to rescue wMel induced CI. Filled sex symbols represent infection with wMel Wolbachia, and gene names to the right of a symbol represent expression of those genes in the corresponding sex of that cross. Vertical bars represent medians. Letters to the right indicate significant differences with an α=0.05. (B) To test if nos-GAL4-tubulin and nos-GAL4:VP16 generate different levels of RNA expression, cifA_{wMel} fold expression difference relative to the Drosophila housekeeping gene rp49 in male abdomens under the two drivers was measured using qPCR. Females tested for gene expression were the same used in the hatch rate experiment in A.

FIG. 14. CI and rescue can be synthetically recapitulated under transgenic expression in the absence of Wolbachia. Single cifA_{wMel} and dual cifA_{wMel} and cifB_{wMel} expression under nos-GAL4:VP16 in uninfected females (open circles) were tested for their ability to rescue transgenic CI under the same driver in uninfected males. Filled sex symbols represent infection with wMel Wolbachia, and gene names beside a symbol represent expression of those genes in the corresponding sex of that cross. Vertical bars represent medians. Letters to the right indicate significant differences with an α=0.05.

FIG. 15. Neither cifA_{wMel} or cifB_{wMel} alone can induce CI when expressed under nos-GAL4:VP16. cifA_{wMel} and cifB_{wMel} were tested for their ability to induce CI individually under nos-GAL4:VP16 expression in uninfected males (open circles). Filled sex symbols represent infection with wMel Wolbachia and gene names to the right of a symbol represent expression of those genes in the corresponding sex of that cross. Vertical bars represent medians. Letters to the right indicate significant differences with an α=0.05.

FIG. 16. The Two-by-One model of CI and its implications for bidirectional incompatibility and vector control. (A) The Two-by-One genetic model explains that cifA and cifB dual expression in uninfected male insects is necessary for embryonic lethality (CI; skull) when crossed to uninfected and non-expressing females. However, females expressing cifA can rescue CI in their offspring (rescue; open circle). (B) Based on this model, the simplest genetic basis for bidirectional CI is through a single mutation in cifA (cifAΔ) that loses compatibility (CI; skull) with the ancestral variant (cifA) but retains compatibility (rescue; open circle) with the mutant. (C) Synthetic replication of CI and rescue enables studies exploring these genes as tools to drive a payload gene capable of reducing the vectoral capacity of a vector into a population. Black insect silhouettes represent wild type insects whereas teal insects carry a vector blocking payload and the CI genes cifA and cifB.

FIG. 17. Fold expression of transgenic cifA_{wMel} in males relative to the Drosophila housekeeping gene rp49 does not correlate with hatch rate under either nos-GAL4 driver. A linear regression of cifA_{wMel} expression and embryonic hatching reveals no correlation for either nos-GAL4-tubulin or nos-GAL4:VP16. Removal of data points corresponding to 0% embryonic hatching did not change the significance of the correlation. This analysis uses hatch rate samples from the experiment in FIG. 1A and expression data from FIG. 1B

FIG. 18. Experimental replicate of FIG. 4 showing that neither cifA_{wMel} or cifB_{wMel} alone can induce CI when expressed under nos-GAL4:VP16. cifA_{wMel} and cifB_{wMel} were tested for their ability to induce CI individually under nos-GAL4:VP16 expression in uninfected males (open circles). Filled sex symbols represent infection with wMel Wolbachia and gene names to the right of a symbol represent expression of those genes in the corresponding sex of that cross. Vertical bars represent medians. Letters to the right indicate significant differences with an α=0.05.

### DETAILED DESCRIPTION

Disclosed herein are improved genetically modified bacteria, genetically modified bacteriophage, and genetically modified arthropods useful for controlling and/or reducing populations of arthropods (for example, insects). As cifA and cifB are the only two wMel genes associated with cytoplasmic incompatibility (CI), it was previously unknown whether the CI induction and rescue genes might be the same. In addition, previous gene drivers did not produce complete CI induction. Here, the inventors have shown that transgenic expression of the cifA gene using the nos-Gal4:VP16 gene driver (or the maternal triple driver (MTD)) from wMel Wolbachia in ovaries was surprisingly found to fully rescue CI and nullify associated embryonic defects. Thus, disclosed herein are improved gene drivers for use in microbial drive systems for vector control.

Reference will now be made in detail to the embodiments of the invention, examples of which are illustrated in the drawings and the examples. This invention may, however, be embodied in many different forms and should not be construed as limited to the embodiments set forth herein.

Unless defined otherwise, all technical and scientific terms used herein have the same meaning as commonly understood to one of ordinary skill in the art to which this disclosure belongs. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed.

### Terminology

The following definitions are provided for the full understanding of terms used in this specification.

As used herein, the article “a,” “an,” and “the” means “at least one,” unless the context in which the article is used clearly indicates otherwise.

The term “nucleic acid” as used herein means a polymer composed of nucleotides, e.g. deoxyribonucleotides or ribonucleotides.

The terms “ribonucleic acid” and “RNA” as used herein mean a polymer composed of ribonucleotides.

The terms “deoxyribonucleic acid” and “DNA” as used herein mean a polymer composed of deoxyribonucleotides.

The term “oligonucleotide” denotes single- or double-stranded nucleotide multimers of from about 2 to up to about 100 nucleotides in length. Suitable oligonucleotides may be prepared by the phosphoramidite method described by Beaucage and Carruthers, Tetrahedron Lett., 22:1859-1862 (1981), or by the triester method according to Matteucci, et al., J. Am. Chem. Soc., 103:3185 (1981), both incorporated herein by reference, or by other chemical methods using either a commercial automated oligonucleotide synthesizer or VLSIPS™ technology. When oligonucleotides are referred to as “double-stranded,” it is understood by those of skill in the art that a pair of oligonucleotides exist in a hydrogen-bonded, helical array typically associated with, for example, DNA. In addition to the 100% complementary form of double-stranded oligonucleotides, the term “double-stranded,” as used herein is also meant to refer to those forms which include such structural features as bulges and loops, described more fully in such biochemistry texts as Stryer, Biochemistry, Third Ed., (1988), incorporated herein by reference for all purposes.

The term “polynucleotide” refers to a single or double stranded polymer composed of nucleotide monomers.

The term “polypeptide” refers to a compound made up of a single chain of D- or L-amino acids or a mixture of D- and L-amino acids joined by peptide bonds.

The term “complementary” refers to the topological compatibility or matching together of interacting surfaces of a probe molecule and its target. Thus, the target and its probe can be described as complementary, and furthermore, the contact surface characteristics are complementary to each other.

The term “hybridization” refers to a process of establishing a non-covalent, sequence-specific interaction between two or more complementary strands of nucleic acids into a single hybrid, which in the case of two strands is referred to as a duplex.

The term “anneal” refers to the process by which a single-stranded nucleic acid sequence pairs by hydrogen bonds to a complementary sequence, forming a double-stranded nucleic acid sequence, including the reformation (renaturation) of complementary strands that were separated by heat (thermally denatured).

The term “melting” refers to the denaturation of a double-stranded nucleic acid sequence due to high temperatures, resulting in the separation of the double strand into two single strands by breaking the hydrogen bonds between the strands.

The term “target” refers to a molecule that has an affinity for a given probe. Targets may be naturally-occurring or man-made molecules. Also, they can be employed in their unaltered state or as aggregates with other species.

The term “promoter” or “regulatory element” refers to a region or sequence determinants located upstream or downstream from the start of transcription and which are involved in recognition and binding of RNA polymerase and other proteins to initiate transcription. Promoters need not be of bacterial origin, for example, promoters derived from viruses or from other organisms can be used in the compositions, systems, or methods described herein. In some embodiments, the promoter is referred to as an “activating site” in the context of GAL4 promotion of UAS transgenes.

A polynucleotide sequence is “heterologous” to a second polynucleotide sequence if it originates from a foreign species, or, if from the same species, is modified by human action from its original form. For example, a promoter operably linked to a heterologous coding sequence refers to a coding sequence from a species different from that from which the promoter was derived, or, if from the same species, a coding sequence which is different from naturally occurring allelic variants.

The term “recombinant” refers to a human manipulated nucleic acid (e.g. polynucleotide) or a copy or complement of a human manipulated nucleic acid (e.g. polynucleotide), or if in reference to a protein (i.e, a “recombinant protein”), a protein encoded by a recombinant nucleic acid (e.g. polynucleotide). In embodiments, a recombinant expression cassette comprising a promoter operably linked to a second nucleic acid (e.g. polynucleotide) may include a promoter that is heterologous to the second nucleic acid (e.g. polynucleotide) as the result of human manipulation (e.g., by methods described in Sambrook et al., Molecular Cloning—A Laboratory Manual, Cold Spring Harbor Laboratory, Cold Spring Harbor, N.Y., (1989) or Current Protocols in Molecular Biology Volumes 1-3, John Wiley & Sons, Inc. (1994-1998)). In another example, a recombinant expression cassette may comprise nucleic acids (e.g. polynucleotides) combined in such a way that the nucleic acids (e.g. polynucleotides) are extremely unlikely to be found in nature. For instance, human manipulated restriction sites or plasmid vector sequences may flank or separate the promoter from the second nucleic acid (e.g. polynucleotide). One of skill will recognize that nucleic acids (e.g. polynucleotides) can be manipulated in many ways and are not limited to the examples above.

The term “expression cassette” refers to a nucleic acid construct, which when introduced into a host cell, results in transcription and/or translation of a RNA or polypeptide, respectively. In embodiments, an expression cassette comprising a promoter operably linked to a second nucleic acid (e.g. polynucleotide) may include a promoter that is heterologous to the second nucleic acid (e.g. polynucleotide) as the result of human manipulation (e.g., by methods described in Sambrook et al., Molecular Cloning—A Laboratory Manual, Cold Spring Harbor Laboratory, Cold Spring Harbor, N.Y., (1989) or Current Protocols in Molecular Biology Volumes 1-3, John Wiley & Sons, Inc. (1994-1998)). In some embodiments, an expression cassette comprising a terminator (or termination sequence) operably linked to a second nucleic acid (e.g. polynucleotide) may include a terminator that is heterologous to the second nucleic acid (e.g. polynucleotide) as the result of human manipulation. In some embodiments, the expression cassette comprises a promoter operably linked to a second nucleic acid (e.g. polynucleotide) and a terminator operably linked to the second nucleic acid (e.g. polynucleotide) as the result of human manipulation. In some embodiments, the expression cassette comprises an endogenous promoter. In some embodiments, the expression cassette comprises an endogenous terminator. In some embodiments, the expression cassette comprises a synthetic (or non-natural) promoter. In some embodiments, the expression cassette comprises a synthetic (or non-natural) terminator.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%,94%, 95%, 96%, 97%, 98%, 99% or higher identity over a specified region when compared and aligned for maximum correspondence over a comparison window or designated region) as measured using a BLAST or BLAST 2.0 sequence comparison algorithms with default parameters described below, or by manual alignment and visual inspection (see, e.g., NCBI web site or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 10 amino acids or 20 nucleotides in length, or more preferably over a region that is 10-50 amino acids or 20-50 nucleotides in length. As used herein, percent (%) amino acid sequence identity is defined as the percentage of amino acids in a candidate sequence that are identical to the amino acids in a reference sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

For sequence comparisons, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

One example of algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al. (1990) J. Mol. Biol. 215:403-410). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always>0) and N (penalty score for mismatching residues; always<0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01.

The phrase “codon optimized” as it refers to genes or coding regions of nucleic acid molecules for the transformation of various hosts, refers to the alteration of codons in the gene or coding regions of polynucleic acid molecules to reflect the typical codon usage of a selected organism without altering the polypeptide encoded by the DNA. Such optimization includes replacing at least one, or more than one, or a significant number, of codons with one or more codons that are more frequently used in the genes of that selected organism. For example, the sequence of a heterologous gene expressed in Wolbachia may be “codon optimized” to optimize gene expression based on the preferred codon usage in Wolbachia; or, for example, the sequence of a heterologous gene expressed in Drosophila may be “codon optimized” to optimize gene expression based on the preferred codon usage in Drosophila.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are near each other, and, in the case of a secretory leader, contiguous and in reading phase. However, operably linked nucleic acids (e.g. enhancers and coding sequences) do not have to be contiguous Linking is accomplished by ligation at convenient restriction sites. If such sites do not exist, the synthetic oligonucleotide adaptors or linkers are used in accordance with conventional practice. In embodiments, a promoter is operably linked with a coding sequence when it is capable of affecting (e.g. modulating relative to the absence of the promoter) the expression of a protein from that coding sequence (i.e., the coding sequence is under the transcriptional control of the promoter).

“Transformation” refers to the transfer of a nucleic acid molecule into a new carrier (e.g. Wolbachia cell or phage or prophage). In embodiments, the nucleic acid molecule may be a plasmid that replicates autonomously or it may integrate into the genome of the host organism. Host organisms containing the transformed nucleic acid molecule may be referred to as “transgenic” or “recombinant” or “transformed” organisms. A “genetically modified” organism (e.g. genetically modified arthropod) is an organism that includes a nucleic acid that has been modified by human intervention. Examples of a nucleic acid that has been modified by human intervention include, but are not limited to, insertions, deletions, mutations, expression nucleic acid constructs (e.g. over-expression or expression from a non-natural promoter or control sequence or an operably linked promoter and gene nucleic acid distinct from a naturally occurring promoter and gene nucleic acid in an organism), extra-chromosomal nucleic acids, and genomically contained modified nucleic acids.

“Transinfection” as used herein refers to extracting a microbe (either a pure extraction or mixed with other organisms or substances) from its natural host and then infecting an unnatural host with the extract. The recipient organism is then transinfected with a foreign microbe.

The term “bacterial operon” as used herein refers to a gene or multiple genes transcribed from a single promoter which leads to the production of a single transcript in which one or more coding regions are linked.

The term “cytoplasmic incompatibility (CI) factor” or “cytoplasmic incompatibility (CI) gene” refers to the genes or the factors encoded by the genes from bacteria which provide a function that is required and/or beneficial to produce the natural genetic drive mechanism of cytoplasmic incompatibility (CI) used by various, unrelated bacterial infections (e.g., Wolbachia and Cardinium endosymbionts). “Cytoplasmic incompatibility (CI) factors” can include those factors that induce the CI and can also include those rescue factors that counteract the CI. In some embodiments, a single bacterial operon may encode multiple cytoplasmic incompatibility (CI) factors. In some embodiments, multiple bacterial genes may encode multiple cytoplasmic incompatibility (CI) factors, wherein each gene is transcribed as an independent RNA transcript. In some embodiments, a single bacterial operon may encode a factor that induces the CI and can also encode a factor that can counteract the CI (for example, a rescue factor).

The term “variant” or “derivative” as used herein refers to an amino acid sequence derived from the amino acid sequence of the parent protein having one or more amino acid substitutions, insertions, and/or deletions. For example, a “cytoplasmic incompatibility (CI) factor variant” includes cytoplasmic incompatibility (CI) factor that may have a number of amino acid changes. In some embodiments, the variants may be greater than about 20%, greater than about 30%, greater than about 40%, greater than about 50%, greater than about 60%, greater than about 70%, greater than about 80%, greater than about 90%, or greater than about 95%, identical to the parent nucleic acid sequence or amino acid sequence.

### Gene Drivers and Genetically Modified Arthropods

In some aspects, disclosed herein is a genetically modified arthropod, said arthropod comprising:
at least one bacterial gene encoding a cytoplasmic incompatibility factor or a variant thereof;
a promoter operably linked to the at least one bacterial gene, wherein the promoter comprises a Gal4 binding site; and
a nos-Gal4:VP16 gene driver;
wherein the expression of the cytoplasmic incompatibility factor in a male arthropod causes a reduction in viable offspring in comparison to a male arthropod lacking the cytoplasmic incompatibility factor.

In some embodiments, the genetically modified arthropod further comprises an additional gene driver. In some embodiments, the additional gene driver is a nos-GAL4-tubulin gene driver. In some embodiments, the additional gene driver is an otu-Gal4:VP16 gene driver. In some embodiments, the genetically modified arthropod further comprises a nos-GAL4-tubulin gene driver and an otu-Gal4:VP16 gene driver.

In some embodiments, a variety of GAL4 drivers can be used in gametogenesis. For example, the nos-GAL4-VP15, NGT40 [also known as nos-GAL4-tubulin, nanos-GAL4-tubulin or simply referred to herein as nos-GAL4 (or nanos-Gal4)], and otu-Gal4 (also known as pCOG-Gal4) drivers are previously disclosed (Table 1, Hudson and Cooley. Methods for studying oogenesis. Methods. 2014 June 15; 68(1): 207-217). Other drivers can include Matα-TubGal4, bam-Gal4, tub-Ga14.

In some embodiments, the genetically modified arthropod comprises the maternal triple driver (MTD-GAL4). MTD-Gal4 contains the P{Gal4-nos.NGT}40 [Tracey, W. D., Jr, Ning, X., Klingler, M., Kramer, S. G. and Gergen, J. P. (2000). Quantitative analysis of gene function in the Drosophila embryo. Genetics 154,273 -284], P{COGGAL4:VP16}[Rorth, P. (1998). Gal4 in the Drosophila female germline Mech. Dev. 78,113 -118], and P{nos-Gal4-VP16}[Van Doren, M., Williamson, A. L. and Lehmann, R. (1998). Regulation of zygotic gene expression in Drosophila primordial germ cells. Curr. Biol. 8, 243-246] germline drivers. Description of the MTD driver is found in Petrella et al. which is incorporated herein by reference in its entirety (Petrella L N, Smith-Leiker T, Cooley L. The Ovhts polyprotein is cleaved to produce fusome and ring canal proteins required for Drosophila oogenesis. Development. 2007; 134:703-12).

The first report of the nos-Gal4:VP16 driver is from Van Doren et al. which is incorporated herein by reference in its entirety (Van Doren, M., Williamson, A. L. and Lehmann, R. (1998). Regulation of zygotic gene expression in Drosophila primordial germ cells. Curr. Biol. 8, 243-246). In some embodiments, the nos-Gal4-VP16 transgene construct contains approximately 700 bp of the nos promoter, the nos 5′ and/or 3′ UTRs, and/or approximately 500 bp of genomic sequence 3′ of nos. In some embodiments, the gene driver in the genetically modified arthropod consists of a single gene driver. In some embodiments, the gene driver in the genetically modified arthropod consists of a nos-Gal4:VP16 gene driver. In some embodiments, the gene driver in the genetically modified arthropod consists of an otu-GAL4:VP16 gene driver.

In some aspects, disclosed herein is a genetically modified arthropod, said arthropod comprising:
at least one bacterial gene encoding a cytoplasmic incompatibility factor or a variant thereof;
a promoter operably linked to the at least one bacterial gene, wherein the promoter comprises a Gal4 binding site; and
a otu-GAL4:VP16 gene driver;
wherein the expression of the cytoplasmic incompatibility factor in a male arthropod causes a reduction in viable offspring in comparison to a male arthropod lacking the cytoplasmic incompatibility factor.

In embodiments herein, the nos-Gal4:VP16 gene driver can be replaced by the otu-GAL4:VP16 gene driver. Thus, the gene driver used can comprise or consist of either the nos-Gal4:VP16 gene driver or the otu-GAL4:VP16 gene driver. In some embodiments, the gene driver used can comprise or consist of both the nos-Gal4:VP16 gene driver and the otu-GAL4:VP16 gene driver.

In some embodiments, the at least one bacterial gene is from Wolbachia. In some embodiments, the at least one bacterial gene is from wMel.

In some embodiments, the at least one bacterial gene is from Cardinium. In some embodiments, the at least one bacterial gene is from Rickettsia. In some embodiments, the at least one bacterial gene encodes a deubiquitylase. In some embodiments, the at least one bacterial gene encodes a nuclease.

In some embodiments, the at least one bacterial gene encodes the cytoplasmic incompatibility factor CifA (from locus WD0631) (SEQ ID NO:1). In some embodiments, the at least one bacterial gene encodes the cytoplasmic incompatibility factor CifB (from locus WD0632) (SEQ ID NO:3). In some embodiments, the at least one bacterial gene encodes the cytoplasmic incompatibility factors CifA (WD0631) and/or CifB (WD0632).

In one embodiment, the amino acid sequence of the cytoplasmic incompatibility factor comprises SEQ ID NO:2 (WD0631). In one embodiment, the amino acid sequence of the cytoplasmic incompatibility factor comprises SEQ ID NO:4 (WD0632). In one embodiment, the cytoplasmic incompatibility factors comprise SEQ ID NO:2 and/or SEQ ID NO:4. In one embodiment, the cytoplasmic incompatibility factors comprise SEQ ID NO:2 and/or SEQ ID NO:4, wherein SEQ ID NO:2 and/or SEQ ID NO:4 have been codon optimized (to produce codon optimized variants).

In one embodiment, the at least one bacterial gene encodes a cytoplasmic incompatibility factor of the amino acid sequence SEQ ID NO:2. In one embodiment, the at least one bacterial gene encodes a cytoplasmic incompatibility factor at least 60% identical (for example, at least 60%, at least 65%, at least 70%, at least 75%, at least 80%, at least 85%, at least 90%, at least 95%, at least 99%) to the amino acid sequence SEQ ID NO:2. In one embodiment, the at least one bacterial gene encodes a cytoplasmic incompatibility factor of the amino acid sequence SEQ ID NO:4. In one embodiment, the at least one bacterial gene encodes a cytoplasmic incompatibility factor at least 60% identical (for example, at least 60%, at least 65%, at least 70%, at least 75%, at least 80%, at least 85%, at least 90%, at least 95%, at least 99%) to the amino acid sequence SEQ ID NO:4.

In one embodiment, the genes encoding the cytoplasmic incompatibility factors are from Wolbachia pipientis, for example, CidA^{wPip} (wPa_0282; SEQ ID NO:5), CidB^{wPip} (wPa_0283; SEQ ID NO:7), CinA^{wPip} (wPa_0294; SEQ ID NO:17), and/or CidB^{wPip} (wPa_0295; SEQ ID NO:19).

In one embodiment, the at least one bacterial gene encodes the cytoplasmic incompatibility factor CidA^{wPip} (wPa_0282; SEQ ID NO:6). In one embodiment, the at least one bacterial gene encodes the cytoplasmic incompatibility factor CidB^{wPip} (wPa_0283; SEQ ID NO:8). In one embodiment, the at least one bacterial gene encodes the cytoplasmic incompatibility factors CidA^{wPip} (wPa_0282) and CidB^{wPip} (wPa_0283). In one embodiment, the at least one bacterial gene encodes the cytoplasmic incompatibility factor CinA^{wPip} (wPa_0294; SEQ ID NO:18). In one embodiment, the at least one bacterial gene encodes the cytoplasmic incompatibility factor CinB^{wPip} (wPa_0295; SEQ ID NO:20). In one embodiment, the at least one bacterial gene encodes the cytoplasmic incompatibility factors CinA^{wPip} (wPa_0294) and CinB^{wPip} (wPa_0295).

In one embodiment, the at least one bacterial gene encodes a cytoplasmic incompatibility factor of the amino acid sequence SEQ ID NO:6. In one embodiment, the at least one bacterial gene encodes a cytoplasmic incompatibility factor at least 60% identical (for example, at least 60%, at least 65%, at least 70%, at least 75%, at least 80%, at least 85%, at least 90%, at least 95%, at least 99%) to the amino acid sequence SEQ ID NO:6. in one embodiment, the at least one bacterial gene encodes a cytoplasmic incompatibility factor of the amino acid sequence SEQ ID NO:8. In one embodiment, the at least one bacterial gene encodes a cytoplasmic incompatibility factor at least 60% identical (for example, at least 60%, at least 65%, at least 70%, at least 75%, at least 80%, at least 85%, at least 90%, at least 95%, at least 99%) to the amino acid sequence SEQ ID NO:8.

In one embodiment, the at least one bacterial gene encodes a cytoplasmic incompatibility factor of the amino acid sequence SEQ ID NO:18. In one embodiment, the at least one bacterial gene encodes a cytoplasmic incompatibility factor at least 60% identical (for example, at least 60%, at least 65%, at least 70%, at least 75%, at least 80%, at least 85%, at least 90%, at least 95%, at least 99%) to the amino acid sequence SEQ ID NO:18. In one embodiment, the at least one bacterial gene encodes a cytoplasmic incompatibility factor of the amino acid sequence SEQ ID NO:20. In one embodiment, the at least one bacterial gene encodes a cytoplasmic incompatibility factor at least 60% identical (for example, at least 60%, at least 65%, at least 70%, at least 75%, at least 80%, at least 85%, at least 90%, at least 95%, at least 99%) to the amino acid sequence SEQ ID NO:20.

Additional examples of cytoplasmic incompatibility factors include homologues of CifA (WD0631) and CifB (WD0632) in prophage WO of additional Wolbachia strains including, but not limited to prophages WOMelB, WOHal, WOSol, WORiB, WOSuziB, WOPipl, WOVitA4, WORiC, WOSuziC, wNo, wVitA, and/or wAlbB.

In some embodiments, the at least one bacterial gene encoding a cytoplasmic incompatibility factor may be codon optimized, without changing the resulting polypeptide sequence. In some embodiments, the codon optimization includes replacing at least one, or more than one, or a significant number, of codons with one or more codons that are more frequently used in the genes of that selected arthropod. For example, the sequence of a at least one bacterial gene or a gene encoding a cytoplasmic incompatibility expressed in, for example, an Aedes mosquito, may be “codon optimized” to optimize gene expression based on the preferred codon usage in Aedes.

Non-limiting examples of Type I bacterial genes/operons, Type II bacterial genes/operons, Type III bacterial genes/operons, and additional homologues are known in the art, for example, in WO/2017/214476, which is hereby incorporated by reference in its entirety. WO/2017/214476, discloses methods of utilizing bacterial genes that induce cytoplasmic incompatibility (CI), and discloses the minimal molecular components from the Wolbachia genome that are sufficient to induce sterility by a transgenic means, independent of the Wolbachia bacterium. In embodiments disclosed herein, it is understood that the use of the term “bacterial gene” can encompass genes that are of bacterial origin, and those genes that may be present in a bacterial organism due to insertion of genes from a phage. In embodiments disclosed herein, the terms nos and nanos are used interchangeably.

In one embodiment, the reduction in viable offspring is greater than 50%. In one embodiment, the reduction in viable offspring is greater than 60%. In one embodiment, the reduction in viable offspring is greater than 70%. In one embodiment, the reduction in viable offspring is greater than 80%. In one embodiment, the reduction in viable offspring is greater than 90%. In one embodiment, the reduction in viable offspring is greater than 95%. In some embodiments, the reduction in viable offspring is greater than 10% (for example at least 10%, at least 20%, at least 30%, at least 40%, at least 50%, at least 60%, at least 70%, at least 80%, at least 90%, at least 100%).

In some embodiments, the arthropod is an insect. In some embodiments, the insect is selected from the genera consisting of Aedes, Culex and Anopheles. In some embodiments, the insect is selected from the group consisting of Aedes albopictus, Aedes aegypti and Aedes polynesiensis. In some embodiments, the insect is Drosophila suzukii.

### Methods of Use

In some aspects, disclosed herein is a method for controlling a population of target arthropods, comprising:
providing at least one bacterial gene encoding a cytoplasmic incompatibility factor or a variant thereof, and a promoter operably linked to the at least one bacterial gene, wherein the promoter comprises a Gal4 binding site;
transforming a population of male arthropods with the at least one bacterial gene, wherein the male arthropods comprise a nos-Gal4:VP16 gene driver; and
releasing the male arthropods amongst a population of target arthropods, wherein the release of the male arthropods reduces the population of target arthropods.

In some embodiments, the male arthropods further comprise an additional gene driver. In some embodiments, the additional gene driver is a nos-GAL4-tubulin gene driver. In some embodiments, the additional gene driver is an otu-Gal4:VP16 gene driver. In some embodiments, the male arthropods further comprise a nos-GAL4-tubulin gene driver and an otu-Gal4:VP16 gene driver.

In embodiments herein, the nos-Gal4:VP16 gene driver can be replaced by the otu-GAL4:VP16 gene driver. Thus, the gene driver used can comprise or consist of either the nos-Gal4:VP16 gene driver or the otu-GAL4:VP16 gene driver. In some embodiments, the gene driver used can comprise or consist of both the nos-Gal4:VP16 gene driver and the otu-GAL4:VP16 gene driver.

In some embodiments, the at least one bacterial gene is from Wolbachia. In some embodiments, the at least one bacterial gene is from wMel.

In some embodiments, the at least one bacterial gene encodes the cytoplasmic incompatibility factor CifA (WD0631). In some embodiments, the at least one bacterial gene encodes the cytoplasmic incompatibility factor CifB (WD0632). In some embodiments, the at least one bacterial gene encodes the cytoplasmic incompatibility factors CifA (WD0631) and CifB (WD0632).

In some embodiments, the at least one bacterial gene is from Wolbachia pipientis. In some embodiments, the at least one bacterial gene encodes the cytoplasmic incompatibility factor CidA^{wPip} (wPa_0282). In some embodiments, the at least one bacterial gene encodes the cytoplasmic incompatibility factor CidB^{wPip} (wPa_0283). In some embodiments, the at least one bacterial gene encodes the cytoplasmic incompatibility factors CidA^{wPip} (wPa_0282) and CidB^{wPip} (wPa_0283). In some embodiments, the at least one bacterial gene encodes the cytoplasmic incompatibility factor CinA^{wPip} (wPa_0294). In some embodiments, the at least one bacterial gene encodes the cytoplasmic incompatibility factor CinB^{wPip} (wPa_0295). In some embodiments, the at least one bacterial gene encodes the cytoplasmic incompatibility factors CinA^{wPip} (wPa_0294) and CinB^{wPip} (wPa_0295).

In some embodiments, the reduction in viable offspring is greater than 50%. In some embodiments, the arthropod is an insect. In some embodiments, the insect is selected from the genera consisting of Aedes, Culex and Anopheles. In some embodiments, the insect is selected from the group consisting of Aedes albopictus, Aedes aegypti and Aedes polynesiensis. In some embodiments, the insect is Drosophila suzukii.

In some embodiments, expression of CifA can provide rescue of cytoplasmic incompatibility (CI).

In some aspects, disclosed herein is method for controlling a population of target arthropods, comprising:
providing a genetically modified bacterium comprising:
   at least one bacterial gene encoding a cytoplasmic incompatibility factor or a variant thereof, and
   a promoter operably linked to the at least one bacterial gene, wherein the promoter comprises a Gal4 binding site;
infecting a population of replacement arthropods with the genetically modified bacterium, wherein the replacement arthropods comprise a nos-Gal4:VP16 gene driver; and
releasing the replacement arthropods amongst a population of target arthropods, wherein the release of the replacement arthropods reduces the population of target arthropods.

In some embodiments, the replacement arthropods further comprise an additional gene driver. In some embodiments, the additional gene driver is a nos-GAL4-tubulin gene driver. In some embodiments, the additional gene driver is an otu-Gal4:VP16 gene driver. In some embodiments, the replacement arthropods further comprise a nos-GAL4-tubulin gene driver and an otu-Gal4:VP16 gene driver.

In some embodiments, the at least one bacterial gene is from Wolbachia. In some embodiments, the at least one bacterial gene is from wMel.

In some embodiments, the at least one bacterial gene encodes the cytoplasmic incompatibility factor CifA (WD0631). In some embodiments, the at least one bacterial gene encodes the cytoplasmic incompatibility factor CifB (WD0632). In some embodiments, the at least one bacterial gene encodes the cytoplasmic incompatibility factors CifA (WD0631) and CifB (WD0632).

In some embodiments, the at least one bacterial gene is from Wolbachia pipientis. In some embodiments, the at least one bacterial gene encodes the cytoplasmic incompatibility factor CidA^{wPip} (wPa_0282). In some embodiments, the at least one bacterial gene encodes the cytoplasmic incompatibility factor CidB^{wPip} (wPa_0283). In some embodiments, the at least one bacterial gene encodes the cytoplasmic incompatibility factors CidA^{wPip} (wPa_0282) and CidB^{wPip} (wPa_0283). In some embodiments, the at least one bacterial gene encodes the cytoplasmic incompatibility factor CinA^{wPip} (wPa_0294). In some embodiments, the at least one bacterial gene encodes the cytoplasmic incompatibility factor CinB^{wPip} (wPa_0295). In some embodiments, the at least one bacterial gene encodes the cytoplasmic incompatibility factors CinA^{wPip} (wPa_0294) and CinB^{wPip} (wPa_0295).

In some embodiments, the reduction in viable offspring is greater than 50%. In some embodiments, the arthropod is an insect. In some embodiments, the insect is selected from the genera consisting of Aedes, Culex and Anopheles. In some embodiments, the insect is selected from the group consisting of Aedes albopictus, Aedes aegypti and Aedes polynesiensis. In some embodiments, the insect is Drosophila suzukii.

In some aspects, disclosed herein is method for controlling a population of target arthropods, comprising:
providing a genetically modified bacteriophage comprising:
   at least one bacterial gene encoding a cytoplasmic incompatibility factor or a variant thereof, and
   a promoter operably linked to the at least one bacterial gene, wherein the promoter comprises a Gal4 binding site;
infecting a population of replacement arthropods with the genetically modified bacteriophage, wherein the replacement arthropods comprise a nos-Gal4:VP16 gene driver; and
releasing the replacement arthropods amongst a population of target arthropods, wherein the release of the replacement arthropods reduces the population of target arthropods. In some embodiments, the bacteriophage comprises bacteriophage WO of Wolbachia.

In some aspects, disclosed herein is a method for controlling a population of target arthropods, comprising:
providing at least one bacterial gene encoding a cytoplasmic incompatibility factor or a variant thereof, and a promoter operably linked to the at least one bacterial gene, wherein the promoter comprises a Gal4 binding site;
genetically transforming a bacteria, phage, or prophage with the at least one bacterial gene encoding the cytoplasmic incompatibility factor or variant thereof operably linked to the promoter;
transinfecting a population of replacement arthropods with the bacteria, phage, or prophage, wherein the replacement arthropods comprise a nos-Gal4:VP16 gene driver; and
releasing the population of replacement arthropods amongst a population of target arthropods, wherein the release of the replacement arthropods reduces the population of target arthropods.

In some embodiments, the release of male arthropods expressing cifA; cifB under nos-GAL4:VP16 can yield population suppression. In some embodiments, the release of both male and female arthropods expressing cifA:cifB under nos:GAL4:VP16 yields population replacement and drive into the population. The latter can be used in conjunction with other transgenic approaches to drive pathogen resistance genes that block disease transmission (e.g., Zika and dengue viruses) into a population. In some embodiments, these approaches can also be conducting by replacing the GAL4 binding sites with a native germline promoter, such as nanos.

In some embodiments, for population replacement with cifA; B genetic drive, the drive system can be genetically linked with any gene(s) that would provide a benefit. Examples can include, but are not limited to, those genes that block disease transmission from arthropods to plants; genes that block disease transmission from arthropods to humans; genes that alter arthropod fitness, lifespan, toxins, biting, etc. to propagate different desired traits through a population.

### Arthropods and Infectious Disease Vectors

The inventors have identified improved methods and improved gene drivers for use in the control of arthropod (for example, insects) pests and disease vectors, such as mosquitoes transmitting the Dengue fever and Zika viruses.

In one embodiment, the arthropod is an insect. In one embodiment, the arthropod is a mosquito. In one embodiment, the mosquito is selected from the genera consisting of Aedes, Culex and Anopheles. In one embodiment, the mosquito is an Aedes mosquito. In one embodiment, the mosquito is an Anopheles mosquito. In one embodiment, the mosquito is a Culex mosquito. In one embodiment, the Aedes mosquito species is selected from the group consisting of Aedes albopictus, Aedes aegypti and Aedes polynesiensis. In one embodiment, the Anopheles mosquito species is Anopheles gambiae. In one embodiment, the Culex mosquito species is Culex pipiens.

In one embodiment, disclosed herein are methods for controlling or reducing populations of insects that transmit human or veterinary pathogens. In one embodiment, the pathogen is selected from dengue virus, Zika virus, a malaria parasite (Plasmodium genus), West Nile virus, yellow fever virus, chikungunya virus, Japanese encephalitis, St. Louis encephalitis and Western and Eastern Equine Encephalitis viruses.

In one embodiment, disclosed herein are methods for controlling or reducing populations of insects that transmit trypanosomes including African sleeping sickness, Chagas disease, and Nagana. In one embodiment, the pathogen is Trypanosoma cruzi. In one embodiment, the pathogen is Trypanosoma brucei. In one embodiment, the insect is of the genus Glossina. In one embodiment, the insect is Glossina morsitans. In one embodiment, the insect is a Tsetse fly. In one embodiment, the insect is a kissing bug. In one embodiment, the insect is of the genus Rodnius. In one embodiment, the insect is Rhodnius prolixus.

In one embodiment, disclosed herein are methods for controlling or reducing populations of arthropods that transmit rickettsioses and pathogens within Anaplasmatacea including Rickettsias rickettsii, africae, parkeri, sibirica, conorii, slovaca, peacockii, philipii, rickettsii Hlp2, heilongjiangensis, japonica, montanensis, massiliae, rhipicephali, amblyommii, helvetica, monacensis, buchneri, hoogstralli, felis, akari, australis, canadensis, prowazekii, typhi, bellii. In one embodiment, the arthropod is a tick. In one embodiment, the arthropod is a tick of the genera Amblyomma, Ixodes, or Rhipicephalus. In one embodiment, the disease is epidemic typhus. In one embodiment, the disease is scrub typhus. In one embodiment, the disease is an Ehrlichiosis. In one embodiment, the pathogen is of the genus Ehrlichia. In one embodiment, the pathogen is of the genus Anaplasma. In one embodiment, the pathogen is of the genus Orientia. In one embodiment, the arthropod is a chigger of the genus Leptotrombidium. In one embodiment, the arthropod is a louse of the genus Pediculus. In one embodiment, the arthropod is a flea of the genus Pulex.

In one embodiment, disclosed herein are methods for controlling sandflies that transmit leishmaniasis. In one embodiment, the insect is of the genus Phlebotomus. In one embodiment, the pathogen is of the genus Leishmania. In one embodiment, the pathogen is Leishmania donovani, Leishmania infantum, or Leishmania Chagasi.

In one embodiment, the insect is of various aphids including: Acyrthosiphon kondoi, Brevicoryne brassicae, Rhopalosiphum maidis, Aphis gossypii, Aphis craccivora, Myzus persicae, Rhopalosiphum padi, Acyrthosiphon pisum, Rhopalosiphum rufiabdominalis, Metopolophium dirhodum, Aphis glycine, Therioaphis trifolii, Lipaphis erysimi, Rhopalosiphum padi.

In one embodiment, disclosed herein are methods for controlling the armyworm agricultural pest species including Leucania convecta, Spodoptera exempta, Spodoptera Mauritia, Spodoptera exigua, Mythimna separate, Leucania stenographa.

In one embodiment, disclosed herein are methods for controlling pests of beans and beets. In one embodiment, the insect is either the Bean fly (Ophiomyia phaseoli), the Bean leafroller (Omiodes diemenalis), the Bean looper or Mocis (Mocis alterna), the Bean podborer (Maruca vitrata), the Bean spider mite (Tetranychus ludeni), the Beet webworm (Spoladea recurvalis), the Large Brown bean bug (Riptortus serripes), the Small Brown bean bug (Melanacanthus scutellaris)

In one embodiment, disclosed herein are methods for controlling the Blue oat mite (Penthaleus major). In one embodiment, the invention is useful for controlling the Brown flea beetle (Chaetocnema sp.). In one embodiment, the invention is useful for controlling the Brown mirid (Creontiades pacificus). In one embodiment, the invention is useful for controlling the Brown shield bug (Dictyotus caenosus). In one embodiment, the invention is useful for controlling the Brown wheat mite (Petrobia latens). In one embodiment, the invention is useful for controlling the Bruchid, Cowpea (Callosobruchus maculatus).

In one embodiment, disclosed herein are methods for controlling pests of Corn including: the Corn aphid (Rhopalosiphum maidis), and the Corn earworm (Helicoverpa armigera).

In one embodiment, the invention is useful for controlling pests of cotton including the Cotton aphid (Aphis gossypii), Cotton bollworm (Helicoverpa armigera), the Cotton harlequin bug (Tectocoris diophthalmus), the Cotton leafhopper (Amrasca terraereginae), the Cotton leafperforator (Bucculatrix gossypii), the Cotton looper (Anomis flava), the Cottonseed bug (Oxycarenus luctuosus), the Cotton seedling thrip (Thrips tabaci),the Cotton tipworm (Crocidosema plebejana), and the Cotton webspinner (Achyra affinitalis).

In one embodiment, disclosed herein are methods for controlling the Diamondback moth (Plutella xylostella). In one embodiment, the invention is useful for controlling the Dried fruit beetle (Carpophilus spp.). In one embodiment, the invention is useful for controlling the Eastern false wireworm (Pterohelaeus spp.). In one embodiment, the invention is useful for controlling the Etiella moth (Etiella behrii). In one embodiment, the invention is useful for controlling the False wireworm (Pterohelaeus and Gonocephalum spp.). In one embodiment, the invention is useful for controlling the Flea beetles, Brown and Redheaded (Chaetocnema and Nisostra sp.). In one embodiment, the invention is useful for controlling the Flower beetle (Carpophilus spp.).

In one embodiment, disclosed herein are methods for controlling various Grasshoppers and locusts including the Grasshopper, Wingless (Phaulacridium vittatum), the Locust, Australian plague (Chortoicetes terminifera), the Locust, Migratory (Locusta migratoria), the Locust, Yellow-winged (Gastrimargus musicus), the Locust, Spur-throated (Austracris (Noamdacris) guttulosa).

In one embodiment, the invention is useful for controlling the Greenhouse whitefly (Trialeurodes vaporariorum). In one embodiment, the invention is useful for controlling the Green peach aphid (Myzus persicae). In one embodiment, the invention is useful for controlling the Green mirid (Creontiades dilutus). In one embodiment, the invention is useful for controlling the Green vegetable bug (Nezara viridula). In one embodiment, the invention is useful for controlling the Green stink bug (Plautia affinis). In one embodiment, the invention is useful for controlling the Grey cluster bug (Nysius clevelandensis). In one embodiment, the invention is useful for controlling the Helicoverpa species (armigera and punctigera).

In one embodiment, disclosed herein are methods for controlling planthoppers. In one embodiment, the insect is the small brown planthopper (Laodelphax striatellus). In one embodiment, the invention is useful for preventing the transmission of crop diseases like Rice White Stripe Virus. In one embodiment, the invention is useful for controlling vectors of plant pathogens.

In one embodiment, disclosed herein are methods for controlling the Jassids and various leafhoppers including the Leafhopper, cotton (Amrasca terraereginae), the Leafhopper, lucerne (Austroasca alfalfae), the Leafhopper, maize (Cicadulina bimaculata), the Leafhopper, vegetable (Austroasca viridigrisea).

In one embodiment, disclosed herein are methods for controlling the Loopers including the Looper, Brown pasture (Ciampa arietaria), the Looper, Castor oil (Achaea janata), the Looper, Cotton (Anomis flava), the Looper, Sugarcane (Mocis frugalis), the Looper, Soybean (Thysanoplusia orichalcea), the Looper, Tobacco (Chrysodeixis argentifera), the Looper, Vegetable (Chrysodeixis eriosoma).

In one embodiment, disclosed herein are methods for controlling various Thrip pests including the Onion Thrip (Thrips tabaci), the Cotton seedling Thrip (Thrips tabaci), the Maize Thrip (Frankliniella williamsi), the Plague Thrip (Thrips imaginis), the tobacco Thrip (Thrips tabaci), the Tomato Thrip (Frankliniella schultzei), the Western flower Thrip (Frankliniella orientalis)

In one embodiment, disclosed herein are methods for controlling various Mite pests including the Mite, Bean spider (Tetranychus ludeni), Mite, Brown wheat (Petrobia latens), Mite, Blue oat (Penthaleus major), Mite, Peanut (Paraplonobia spp.), Mite, Redlegged earth (Halotydeus destructor), Mite, Strawberry spider (Tetranychus lambi), and the Two-spotted mite (Tetranychus urticae).

In one embodiment, disclosed herein are methods for controlling various whitefly pests including the Greenhouse whitefly (Trialeurodes vaporariorum), the Silverleaf whitefly (Bemisia tabaci biotype B and Australian native AN), and the Silverleaf whitefly (Bemisia tabaci biotype Q).

In one embodiment, disclosed herein are methods for controlling various fruit pests. In one embodiment, the arthropod is from the genera Drosophila. In one embodiment, the arthropod is Drosophila suzukii. In one embodiment, the arthropod is Drosophila recens, Drosophila subquinaria, Drosophila innubila, or related Drosophila species. Drosophila suzukii, commonly called the spotted-wing drosophila, is a vinegar fly closely related to Drosophila melanogaster. Unlike its vinegar fly relatives who are primarily attracted to rotting or fermented fruit, D. suzukii attacks fresh, ripe fruit by laying eggs under the soft skin. The larvae hatch and grow in the fruit, destroying the fruit's commercial value. The pest particularly (but not limited to) infests cherries, apples, apricots, persimmons, tomatoes, blueberries, grapes, nectarines, pears, plums, peaches, figs, raspberries and strawberries. Although D. suzukii is native to Southeast Asia, the fruit pest has recently invaded North and Central America as well as Europe, where it is expanding rapidly. Effective management of this pest is a challenge owing to the wide host range and short generation time. Therefore, monitoring and controlling D. suzukii is of great economic importance. However, traps and baits containing for instance apple cider vinegar, which are typically used for attracting vinegar flies such as D. melanogaster, are less efficient for attracting and trapping D. suzukii. In one embodiment, the insect is the Mexican Fruit Fly (Anastrepha ludens). In one embodiment, the insect is the Mediterranean Fruit Fly (Ceratitis capitata). In one embodiment, the insect is of the genus Anastrepha, Bactrocera, or Ceratitis. In one embodiment, the insect is a tephritid.

In one embodiment, disclosed herein are methods for controlling various other agricultural pests including: the red-houldered leaf beetle (Monolepta australis), Native budworm (Helicoverpa punctigera), Native whitefly (Bemisia tabaci), Northern armyworm (Mythimna separata), Oat aphid (Rhopalosiphum padi), Onion thrip (Thrips tabaci), Pale cotton stainer bug (Dysdercus sidae), Pea aphid (Acyrthosiphon pisum), Pea blue butterfly (Lampides boeticus), Peanut mite (Paraplonobia spp.), Peanut scarab (Heteronyx spp.), Pea weevil (Bruchus pisorum), Pinkspotted bollworm (Pectinophora scutigera), Plague thrip (Thrips imaginis), Podsucking bugs (Nezara viridula), Redbanded shield bug (Piezodorus oceanicus), Redheaded flea beetle (Nisotra sp.), Redlegged earth mite (Halotydeus destructor), Redshouldered leaf beetle (Monolepta australis), Rice root aphid (Rhopalosiphum rufiabdominalis), Rose grain aphid (Metopolophium dirhodum), Rough bollworm (Earias huegeliana), Rutherglen bug (Nysius vinitor), Seed harvesting ants (Pheidole spp.), Scarab, Black sunflower (Pseudoheteronyx sp.), Scarab, Peanut (JPG, 20.4KB) (Heteronyx sp.), Shoot flies (Atherigona sp.), Silverleaf whitefly (Bemisia tabaci biotype B and Australian native AN), Silverleaf whitefly (Bemisia tabaci biotype Q), Sitona weevil (Sitona discoideus), Solenopsis mealybug (Phenacoccus solenopsis), Sorghum midge (Stenodiplosis sorghicola), Sorghum head caterpillar (Cryptoblabes adoceta), Soybean leafminer (Porphyrosela aglaozona), Soybean looper (Thysanoplusia orichalcea), Soybean moth (Aproaerema simplexella), Spotted alfalfa aphid (Therioaphis trifolii), Spur-throated locust (Austracris (Nomadacris) guttulosa), Strawberry spider mite (Tetranychus lambi), Swarming leaf beetle (Rhyparida spp.), Tortrix (Epiphyasa postvittana), True wireworm (Agrypnus spp.), Vegetable weevil (Listroderes difficilis), Weed web moth (Achyra affinitalis), Whitegrub (Heteronyx spp.), Wingless cockroaches (Calolampra spp.), Wireworm, False (Pterohelaeus and Gonocephalum spp.), Wireworm, True (Agrypnus spp.), Yellow peach moth (Conogethes punctiferalis). In one embodiment, the insect is Heteronychus arator. In one embodiment, the insect is of the genus Amnemus. In one embodiment, the insect is of the genus Pheidole. In one embodiment, the invention is useful for controlling the Black field cricket (Teleogryllus commodus, T. oceanicus, Lepidogryllus parvulus), the Black field earwig (Nala lividipes), the Black leaf beetle (Rhyparida nitida), the Black sunflower scarab (Pseudoheteronyx sp.). In one embodiment, the invention is useful for controlling the Cowpea bruchid (Callosobruchus maculatus). In one embodiment, the invention is useful for controlling the Cricket, Black field (Teleogryllus commodus, T. oceanicus, Lepidogryllus parvulus). In one embodiment, the invention is useful for controlling the Crop mirid (Sidnia kinbergi). In one embodiment, the invention is useful for controlling the Cutworm (Agrotis spp.). In one embodiment, the invention is useful for controlling the Cabbage moth (Plutella xylostella). In one embodiment, the invention is useful for controlling the Castor oil looper (Achaea janata). In one embodiment, the invention is useful for controlling the Click beetle (Agrypnus spp.). In one embodiment, the invention is useful for controlling the Clover springtail (Sminthurus viridis). In one embodiment, the invention is useful for controlling the Cluster caterpillar (Spodoptera litura). In one embodiment, the invention is useful for controlling the Cockroach, Wingless (Calolampra spp.). In one embodiment, the invention is useful for controlling the Common grass blue butterfly (Zizina labradus). In one embodiment, the invention is useful for controlling the Legume webspinner (Omiodes diemenalis). In one embodiment, the invention is useful for controlling the Light brown apple moth (Epiphyas postvittana). In one embodiment, the invention is useful for controlling Mocis trifasciata. In one embodiment, the invention is useful for controlling Pantydia spp. In one embodiment, the invention is useful for controlling the Lucerne crownborer (Zygrita diva). In one embodiment, the invention is useful for controlling the Lucerne flea (Sminthurus viridis). In one embodiment, the invention is useful for controlling the Lucerne leafhopper (Austroasca alfalfae). In one embodiment, the invention is useful for controlling the Lucerne leafroller (Merophyas divulsana). In one embodiment, the invention is useful for controlling the Lucerne seed wasp (Bruchophagus roddi). In one embodiment, the invention is useful for controlling the Lucerne seed web moth (Etiella behrii).

In one embodiment, disclosed herein are methods for controlling forestry and wildlife pests such as the emerald ash borer. In one embodiment, the insect is of the genus Agrilus or specifically Agrilus planipennis. In one embodiment, the invention is useful for pests of trees and lumber.

In one embodiment, disclosed herein are methods for controlling various arthropods including Adalia bipunctata (two-spotted lady beetle), other ladybug species/genera (Harmonia, Adalia decempunctata, Cadra cautella (and other Cadra moths), Ephestia kuehniella (and other Ephestia moths), Cordylochernes scorpioides (pseudoscorpion), Tribolium (flour beetles), Hypolimnas butterflies, Acraea butterflies, or Ostrinia moths.

### EXAMPLES

The following examples are set forth below to illustrate the compositions, methods, and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

### Example 1. A single Prophage WO Gene Rescues Cytoplasmic Incompatibility in Drosophila melanogaster

Wolbachia are maternally-inherited, intracellular bacteria at the forefront of vector control efforts to curb arbovirus transmission. In international field trials, the cytoplasmic incompatibility (CI) drive system of wMel Wolbachia is deployed to replace target vector populations, whereby a Wolbachia-induced modification of the sperm genome kills embryos. However, Wolbachia in the embryo rescue the sperm genome impairment, and therefore CI results in a strong fitness advantage for infected females that transmit the bacteria to offspring. The two genes responsible for the wMel-induced sperm modification of CI, cifA and cifB, were recently identified in the eukaryotic association module of prophage WO, but the genetic basis of rescue is unresolved. Here, transgenic and cytological approaches were used to demonstrate that cifA independently rescues CI and nullifies embryonic death caused by wMel Wolbachia in Drosophila melanogaster. Discovery of cifA as the rescue gene and previously one of two CI induction genes establishes a new ‘Two-by-One’ model that underpins the genetic basis of CI. Results highlight the central role of prophage WO in shaping Wolbachia phenotypes that are significant to arthropod evolution and vector control.

The World Health Organization recommended pilot deployment of Wolbachia-infected mosquitoes to curb viral transmission to humans. Releases of mosquitoes are underway worldwide because Wolbachia can block replication of these pathogenic viruses and deterministically spread by a drive system termed cytoplasmic incompatibility (CI). Despite extensive research, the underlying genetic basis of CI remains only half-solved. It was recently reported that two prophage WO genes recapitulate the modification component of CI in a released strain for vector control. Here, it is shown that one of these genes underpins rescue of CI. Together, these results reveal the complete genetic basis of this selfish trait and provides an alternative to current control efforts.

### Background

Wolbachia are an archetype of maternally-inherited, intracellular bacteria. They occur in an estimated 40-52% of arthropod species (1, 2) and 47% of the Onchocercidae family of filarial nematodes (3), making them the most widespread bacterial symbiont in the animal kingdom (2). In arthropods, Wolbachia mainly reside in the cells of the reproductive tissues, transmit transovarially (4), and often commandeer host fertility, sex ratios, and sex determination to enhance their maternal transmission via male-killing, feminization, parthenogenesis, or cytoplasmic incompatibility (CI) (5, 6).

Discovered nearly half a century ago (7), Wolbachia-induced CI is the most common reproductive modification and results in embryonic lethality when an infected male mates with an uninfected female, but this lethality is rescued when the female is likewise infected (8). As such, rescue provides a strong fitness advantage to infected females, the transmitting sex of Wolbachia (9-11). Alone, CI-induced lethality is deployed in vector control studies to crash the resident uninfected mosquito population through release of Wolbachia-infected males (12-17). Together, CI-induced lethality and rescue constitute a microbial drive system that is used in field studies worldwide to stably replace an uninfected mosquito population with an infected one via release of male and females harboring wMel Wolbachia (18), which confer resistance against dengue and Zika viruses (19, 20). The efficacy of this drive system for spreading Wolbachia in target populations critically depends on Wolbachia's ability to rescue its own lethal modification of the sperm.

While CI is gaining momentum as a natural, sustainable, and inexpensive tool for vector control, the genes that underpin this microbial adaptation are not fully known. A previous screen of Wolbachia genomes and transcriptomes from infected ovaries identified two adjacent genes, cifA and cifB, from the wMel strain in Drosophila melanogaster as the only genes consistently associated with CI (21). These two genes occur in the eukaryotic association module of prophage WO (22), and they together recapitulate CI when dually expressed in uninfected male flies (21, 23). Each gene alone is incapable of inducing CI (21), and the rescue gene remains unknown. As cifA and cifB are the only two wMel genes associated with CI, it was previously unknown whether the CI induction and rescue genes might be the same (21). Here, transgenic expression of cifA and/or cifB genes from wMel Wolbachia in ovaries was investigated to determine if these genes rescue CI and nullify the associated embryonic defects in D. melanogaster.

### Results and Discussion

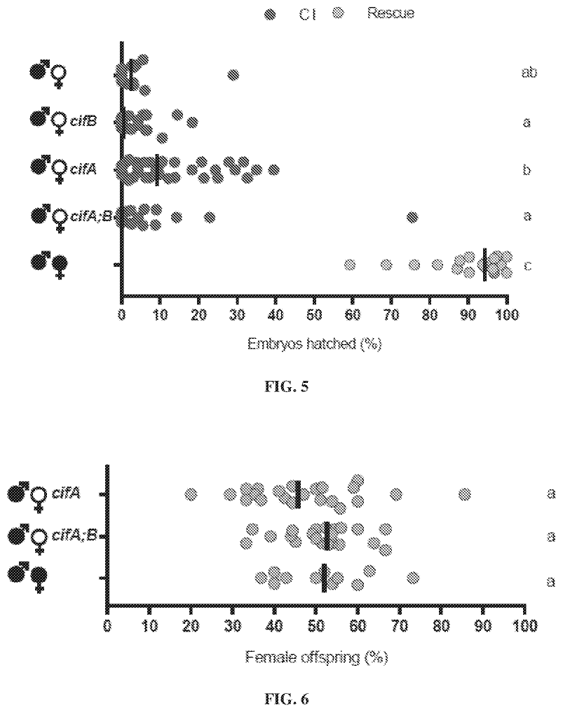

Since Wolbachia cannot be genetically transformed, the ability of cifA to transgenically rescue wild type CI was tested using a GAL4-UAS system for tissue-specific expression in uninfected D. melanogaster females. As such, the transgenic experiments were conducted under the control of either nos-GAL4-tubulin in uninfected germline stem cells or maternal triple driver, MTD-GAL4, to drive higher transgene expression throughout oogenesis. In transcriptomes of wMel-infected D. melanogaster, cifA is a highly expressed prophage WO gene (24). MTD-GAL4 utilizes two nos-GAL4 driver variants (including nos-GAL4-tubulin) and an ovarian tumor driver (25). Control CI and rescue crosses with either driver yielded the expected hatching rates. Crosses between infected males and uninfected females expressing cifA under the control of MTD-GAL4 showed a markedly significant increase in embryonic hatching relative to cifA expression under nos-GAL4-tubulin and at levels similar to that in control rescue crosses (FIG. 1A). These results are consistent with complete rescue of CI by cifA, in association with increased expression throughout the developing egg chambers. Similar results with nos-GAL4-tubulin expression in uninfected ovarian germline stem cells resulted in a small increase in hatch rate that was inconsistently significant among replicates (FIG. 5). An analysis of cifA gene expression reveals MTD-GAL4 associates with a surprising three-order-of-magnitude increase over nos-GAL4-tubulin, supporting strength of expression as a factor for rescue (FIG. 1B).

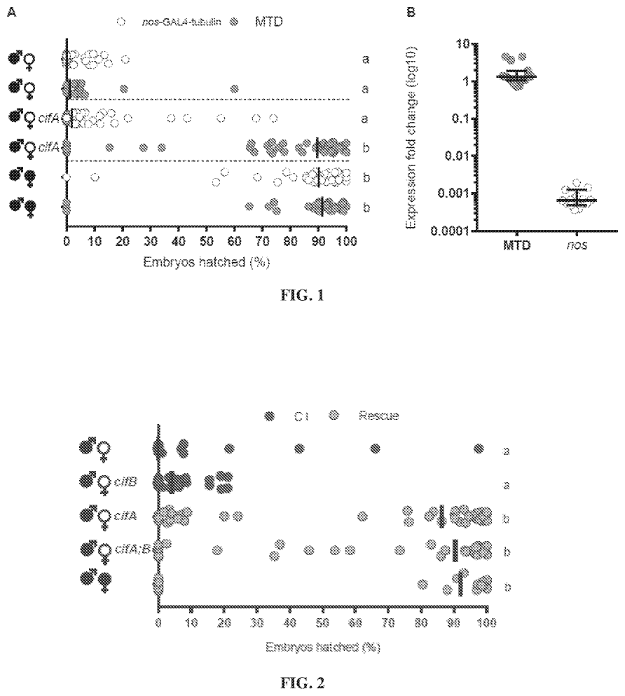

Gene expression of cif was also evaluated under the control of MTD-GAL4 in uninfected females to test if cifB alone or in combination with cifA impacts CI penetrance. Infected males crossed to either uninfected females or females transgenically expressing cifB under MTD-GAL4 yielded similar CI penetrance (FIG. 2). These results suggest that cifB does not rescue CI when transgenically expressed in the ovaries, and its CI-related function is specific to testes. In contrast, MTD-GAL4 expression of cifA, by itself or in combination with cifB, significantly rescued CI to levels comparable to rescue by infected females (FIG. 2). These results are consistent with cifA independently functioning as the rescue factor and suggest that cifB does not inhibit cifA's ability to rescue CI. As Wolbachia can induce phenotypes known to bias sex ratios, the surviving offspring were collected from the transgenic and control rescue crosses and sexed them to demonstrate normal sex ratios, indicating that rescue was not sex-specific (FIG. 6).

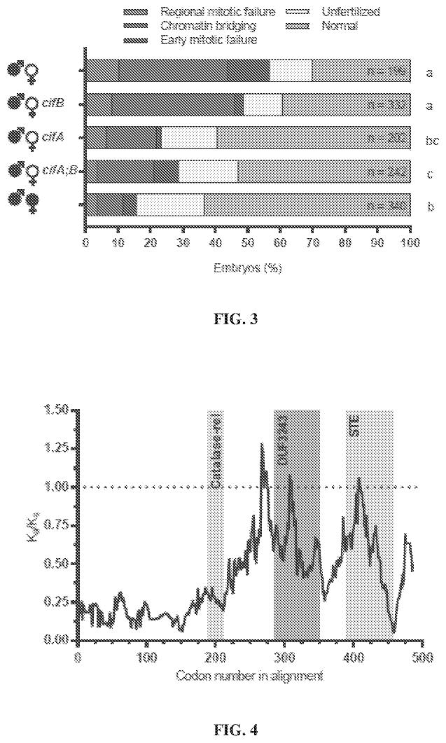

Next, it was tested if the canonical cytological defects observed in early CI embryos (early mitotic failure, chromatin bridging, and regional mitotic failure (26)) were nullified under cifA-induced rescue. Embryos were examined from control and transgenic crosses after 1-2 h of development and binned their cytology into one of five phenotypes as previously established for D. melanogaster CI (21). Nearly half of CI-induced lethality in embryos is the result of embryonic arrest during advanced developmental stages in Dipteran species (27-30). The control CI cross yielded high levels of all three CI-associated defects, and the embryos from the control rescue cross developed with significantly fewer abnormalities (FIG. 3). MTD-GAL4 transgene expression of cifA in uninfected females, either alone or dually expressed with cifB, resulted in significantly fewer cytological defects (FIG. 3). These effects were not seen with transgene cifB expression, again validating that cifA alone can recapitulate wild type rescue by Wolbachia.

These data are in contrast with previous work reporting the inability to transgenically rescue CI in D. melanogaster (23); however, there are three critical differences between the studies. First, wPip's homologs from Culex pipiens were used in the prior work instead of wMel's cif genes from D. melanogaster here. Thus, differences in host background interactions could explain the discrepancy. Second, a T2A sequence for the wPip gene homologs was used to allow for bicistronic expression, but ribosome skipping results in a C-terminal sequence extension to the first protein and a proline addition to the second protein that generates sequence artifacts and could alter function (31). Finally, different insertion sites are capable of different levels of expression due to their local chromatin environment (32), thus the chosen sites may produce insufficient product to cause rescue, as was the case when cifA was driven by nos-GAL4-tubulin.

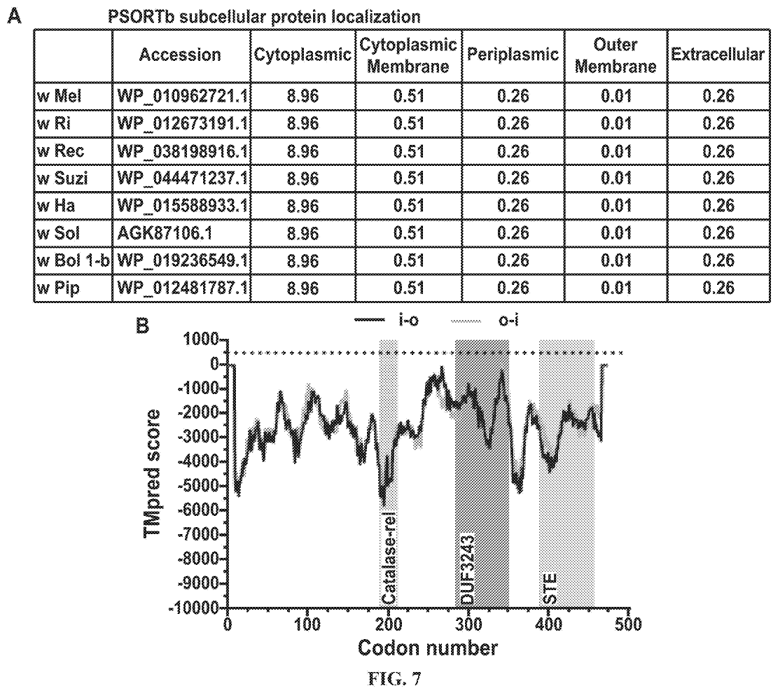

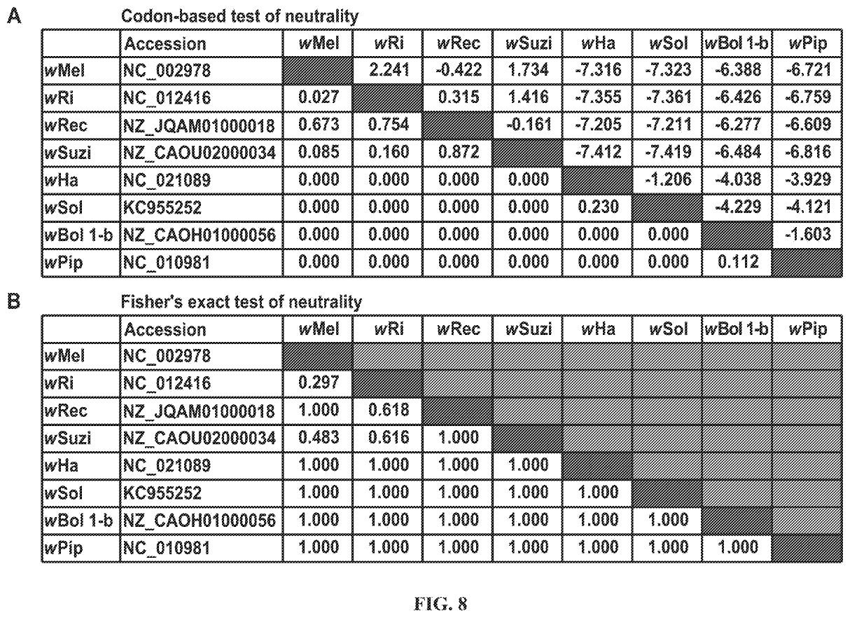

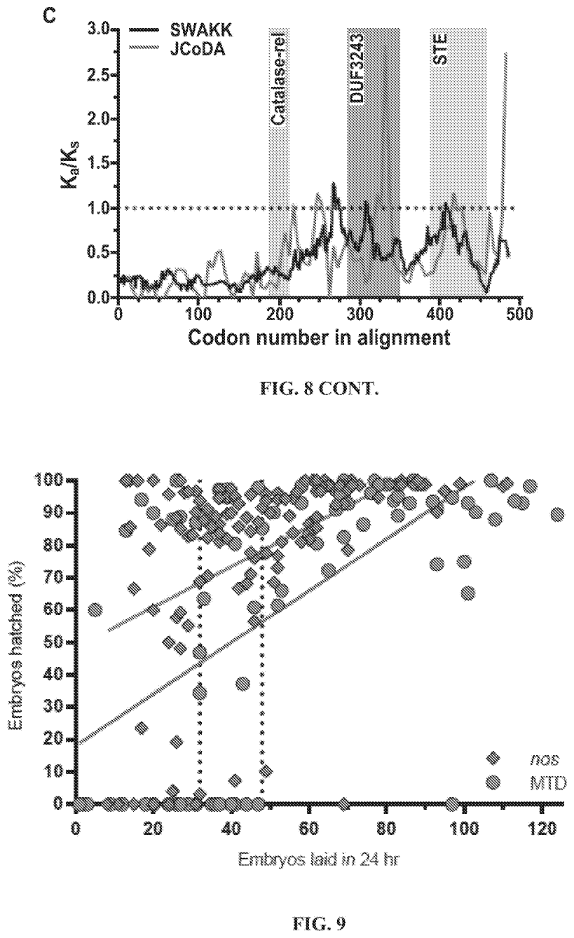

cifA encodes a putative catalase-rel function, sterile-like transcription factor (STE) domains, and a domain of unknown function (DUF3243) that shares homology with a putative Puf-family RNA binding domain in cifA-like homologs (33), whereas cifB has nuclease and deubiquitilase domains (23, 33). Only the deubiquitilase annotation has been functionally tested and confirmed(23). Based on subcellular localization (PSORTb) and transmembrane helix predictors (TMbase), CifA is a cytoplasmic protein without transmembrane helices (FIG. 7). Codon-based and Fisher's exact tests of neutrality demonstrate that closely-related (76.2-99.8% pairwise nucleotide identity) Type I CifA homologs (21) largely evolve by purifying selection (FIG. 8a, b), and sliding window analyses (SWAKK and JCoDA) reveal that purifying selection is strongest on the catalase-rel domain and the unannotated region at the N-terminus, with considerably weaker purifying selection on the putative DUF3243 and STE domains (FIG. 4; FIG. 8c). This is supported by prior work reporting stronger amino acid conservation within the Type I CifA N-terminus relative to the C-terminus (33).

These findings illustrate that the Wolbachia prophage WO gene cifA recapitulates rescue of wild type CI. As cifA is one of two genes involved in induction of CI, results support the hypothesis that a gene involved in CI induction is also the rescue gene (21). In addition, transgenic expression of cifA in yeast inhibits a temperature-dependent growth defect caused by cifB expression (23). The discovery that CI is induced by cifA and cifB and rescued by cifA motivates a new modification-rescue model of CI where two genes act as the CI modification factors (in the male), and one of these same genes acts as the rescue factor (in the female). This ‘Two-by-One’ model posits that each strain of Wolbachia has its own set of cifA- and cifB-associated CI modifications and one cifA rescue factor. The different roles of cifA in CI and rescue is intriguing. The function of cifA is likely dependent on differential tissue localization of gene products in male and female reproductive systems and/or alternate post-translational modification in testes/sperm (CI) versus in ovaries/embryos (rescue). Moreover, one could speculate that the putative antioxidant catalase-rel domain of the CifA protein acts as a functional switch in the presence of reactive oxygen species, known to be higher in Wolbachia-infected testes (34), whereas the Puf-family RNA binding domain and STE are involved in RNA binding and transcriptional (mis)regulation of an unknown host factor.

It has been hypothesized that divergence in modification and rescue genes leads to bidirectional CI (21, 37, 38), which is a reciprocal incompatibility between males and females infected with different Wolbachia strains (7, 39-42). Comparative genomic analyses of cifA and cifB genes reveal extremely high levels of amino acid divergence (21), strong codivergence (21, 33), and recombination (38), consistent with the very rapid evolution of bidirectional CI across Wolbachia that can contribute to reproductive isolation and speciation (42, 43). Indeed, divergence of the cifA and cifB genes into several phylogenetic types correlates with bidirectional CI patterns in Drosophila and Culex (21, 38). There are at least two explanations for how simple genetic changes in these genes can contribute to bidirectional CI. First, a single mutation in the cifA gene could produce variation in the modification and rescue components that render two Wolbachia strains incompatible. For instance, given an ancestral and derived allele of cifA, males and females with Wolbachia carrying the same cifA allele are compatible; however, males with Wolbachia carrying the ancestral cifA allele cause a sperm modification that is unable to be rescued by embryos with Wolbachia carrying the derived cifA allele, and vice versa. Thus, a single mutation in cifA alone can enable the switch from being compatible to incompatible Wolbachia. Second, mutations in both cifA and cifB are required for the evolution of bidirectional CI. For example, CifA-CifB protein binding (23) and/or differential localization in the sperm and egg may underpin bidirectional CI between Wolbachia strains. In this model, amino acid divergence in the Cif proteins may contribute to weakened binding, which in turn yields Wolbachia strains incapable of CI but capable of rescuing the ancestral variant (44, 45). A compensatory substitution in the other Cif protein could in theory restore binding and yield bidirectional incompatibility with the ancestral Cif variants. Codivergence between amino acid sequences of these proteins is consistent with this model. Under both models, the presence of multiple WO prophages carrying cifA genes may also promote incompatibilities through the production of multiple CI product complexes simultaneously (21). In support of these hypotheses, complex diversification and duplication of cifA and cifB has been reported in Drosophila and C. pipiens that harbor a variety of incompatible Wolbachia strains (21, 38).

In conclusion, these findings reveal the connected genetic basis of CI and rescue and highlight the fundamental impact of prophage genes on the adaptive phenotypes of an obligate intracellular bacteria. In addition to genetically dissecting this widespread form of reproductive parasitism and microbial drive, a new Two-by-One model was established to explain the modification and rescue components of CI. Finally, the constructs and methods herein are used as transgenic drive constructs and/or as adjuncts or alternatives to pest control or vector control strategies currently deploying Wolbachia-infected mosquitoes (15-18).

### Materials and Methods

Fly rearing and strains. D. melanogaster stocks y¹w* (BDSC 1495), nos-GAL4-tubulin (BDSC 4442), MTD-GAL4 (containing nos-GAL4-tubulin, nos-GAL4-VP16, and otu-GAL4-VP16; BDSC 31777), and UAS transgenic lines homozygous for cifA, cifB, and cifA; B (21) were maintained at 12:12 light:dark at 25° C. and 70% relative humidity (RH) on 50 ml of a standard media. GAL4 lines were found to be infected with wMel Wolbachia, and uninfected lines were produced through tetracycline treatment as previously described (21). Infection status was frequently confirmed via PCR using WolbF and WolbR3 primers (46). During virgin collections, flies were stored at 18° C. overnight to slow eclosion rate, and virgin flies were kept at room temperature.

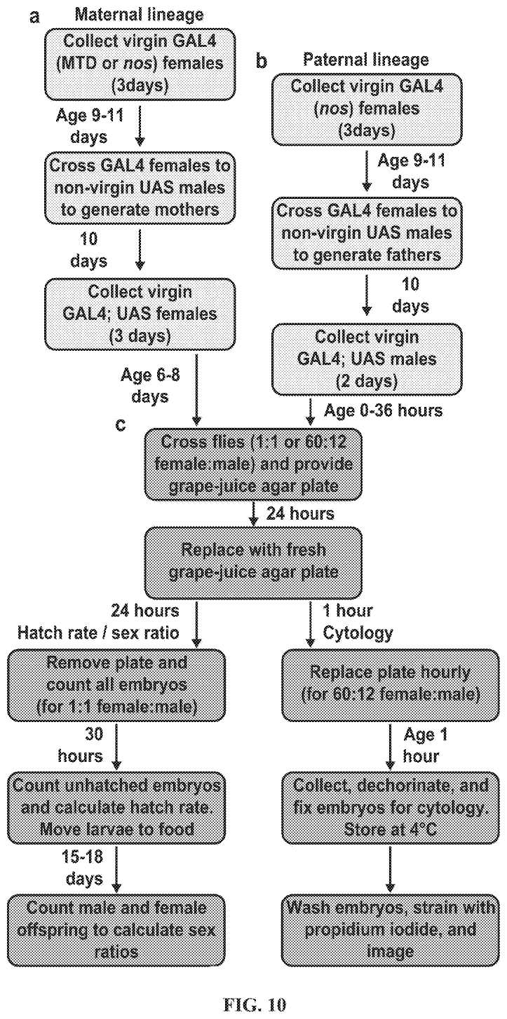

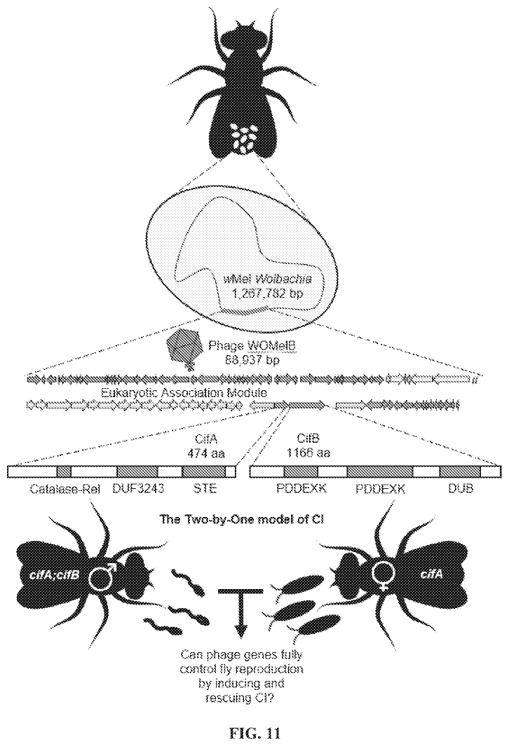

Hatch rate and sex ratio assays. Virgin MTD-GAL4 females were collected for the first 3 days of emergence and aged 9-11 days before crossing to non-virgin homozygous UAS (cifA, cifB, or cifA; B) males. The start of collections for the maternal and paternal lineages were staggered by 7 days. Single pair matings occurred in an 8 oz bottle, and a grape-juice agar plate was smeared with yeast and affixed to the opening with tape. The flies and bottles were then stored at 25° C. and 70% RH for 24 h at which time the plates were replaced with freshly smeared plates and again stored for 24 h. Plates were then removed and the number of embryos on each plate were counted and stored. After 30 h the remaining unhatched embryos were counted (Extended Data FIG. 6). The hatch rate was calculated by dividing the number of hatched embryos by the initial embryo count and multiplying by 100. Hatch rate was plotted against clutch size for all rescue crosses conducted in this study to reveal a significant correlation (FIG. 9), and a threshold clutch size for analysis was set equal to exclusion of 99% of plates with a hatch rate of 0 for each genotype (31 for nos-GAL4-tubulin and 48 for MTD-GAL4). Larvae were moved into vials of standard media and the offspring sex ratio determined after 15-18 days (FIG. 10). Hatch rates testing MTD-GAL4 or nos-GAL4-tubulin expression of cifA were conducted three and four times respectively. Sex ratio experiments were conducted once.

Gene expression. To compare the level of UAS-cifA expression between MTD-GAL4 and nos-GAL4-tubulin flies, mothers from hatch rate assays were collected after the allotted laying period, abdomens were immediately dissected, and samples were frozen in liquid nitrogen and stored at −80C until processing. RNA was extracted using the Direct-zol RNA MiniPrep Kit (Zymo), DNase treated with DNA-free (Ambion, Life Technologies), and cDNA was generated with SuperScript VILO (Invitrogen). Quantitative PCR was performed on a Bio-Rad CFX-96 Real-Time System using iTaq Universal SYBR Green Supermix (Bio-Rad). Forty cycles of PCR were performed against positive controls (extracted DNA), negative controls (water), RNA, and cDNA with the following conditions: 50° C. 10 min, 95° C. 5 min, 40×(95° C. 10 s, 55° C. 30 s), 95° C. 30 s. Primers used were cifA opt and Rp49 forward and reverse. Fold expression of UAS-cifA relative to the D. melanogaster house-keeping gene Rp49 was determined with 2^{-ΔΔCt}. This experiment and corresponding hatch rate were performed once.

Embryo cytology. Flies were collected as described for the hatch rate assays, but with 60 females and 12 males in each bottle with a grape-juice agar plate attached. All flies used were siblings of those from the hatch rate, grape-juice plates replaced as described above, and embryos collected in parallel to egg-laying by hatch rate females. Embryos were collected, dechorionated, washed, methanol fixed, stained with propidium iodide, imaged, and categorized as previously described (21) (FIG. 10). This experiment was performed once.

Putative cifA localization. The PSORTb v3.0.2 web server (47) was used to predict subcellular localization of the wMel CifA protein to either the cytoplasm, cytoplasmic membrane, periplasm, outer membrane, or extracellular space. A localization score is provided for each location with scores of 7.5 or greater considered probable localizations. The TMpred web server (48) was used to predict transmembrane helices in wMel CifA. TMpred scores were generated for transmembrane helices spanning from inside-to-outside (i-o) and outside-to-inside (o-i), and scores above 500 are considered significant.

cifA selection analyses. Selection analyses were conducted using four independent tests of selection: codon-based Z-test of neutrality (49), Fisher's exact test of neutrality (49), Sliding Window Analysis of Ka and Ks (SWAKK) (50), and Java Codon Delimited Alignment (JCoDA) (51). The first two analyses were conducted using the MEGA7 desktop app with a MUSCLE translation alignment generated in Geneious v5.5.9. The SWAKK 2.1 web server and the JCoDA v1.4 desktop app were used to analyze divergence between wMel and wHa cifA with a sliding window of 25 or 50 codons and a jump size of 1 codon for SWAKK and 5 codons for JCoDA.

Statistical analyses. All statistical analyses were conducted in GraphPad Prism (Prism 7 or online tools). Hatch rate and sex ratio statistical comparisons were made using Kruskal-Wallis followed by a Dunn's multiple comparison test. Expression was compared using a Mann-Whitney test. Correlations between hatch rate and clutch size were determined using Spearman rho. Pair-wise chi-square analyses were used for cytology studies to compare defective and normal embryos followed by generation of Bonferroni adjusted p-values. An unpaired t-test was used for statistical comparison of RNA fold expression.

### REFERENCES CITED IN THIS EXAMPLE

1. Weinert L A, Araujo-Jnr E V, Ahmed M Z, & Welch J J (2015) The incidence of bacterial endosymbionts in terrestrial arthropods. Proc Biol Sci 282(1807):20150249.
2. Zug R & Hammerstein P (2012) Still a host of hosts for Wolbachia: analysis of recent data suggests that 40% of terrestrial arthropod species are infected. PLoS One 7(6):e38544.
3. Ferri E, et al. (2011) New insights into the evolution of Wolbachia infections in filarial nematodes inferred from a large range of screened species. PLoS One 6(6):e20843.
4. Frydman H M, Li J M, Robson D N, & Wieschaus E (2006) Somatic stem cell niche tropism in Wolbachia. Nature 441(7092):509-512.
5. Werren J H, Baldo L, & Clark M E (2008) Wolbachia: master manipulators of invertebrate biology. Nat Rev Microbiol 6(10):741-751.
6. LePage D & Bordenstein S R (2013) Wolbachia: Can we save lives with a great pandemic? Trends in parasitology 29(8):385-393.
7. Yen J & Barb A (1973) The etiological agent of cytoplasmic incompatability in Culex pipiens. Journal of Invertebrate Pathology 22.
8. Serbus L R, Casper-Lindley C, Landmann F, & Sullivan W (2008) The genetics and cell biology of Wolbachia-host interactions. Annu Rev Genet 42:683-707.
9. Turelli M & Hoffmann A A (1999) Microbe-induced cytoplasmic incompatibility as a mechanism for introducing transgenes into arthropod populations. Insect Mol Biol 8(2):243-255.
10. Hancock P A, Sinkins S P, & Godfray H C (2011) Strategies for introducing Wolbachia to reduce transmission of mosquito-borne diseases. PLoS Negl Trop Dis 5(4):e1024.
11. Hancock P A, Sinkins S P, & Godfray H C (2011) Population dynamic models of the spread of Wolbachia. Am Nat 177(3):323-333.
12. Atyame C M, et al. (2011) Cytoplasmic incompatibility as a means of controlling Culex pipiens quinquefasciatus mosquito in the islands of the south-western Indian Ocean. PLoS Negl Trop Dis 5(12):e1440.
13. Dobson S L, Bordenstein S R, & Rose R I (2016) Wolbachia mosquito control: Regulated. Science 352(6285):526-527.
14. O'Connor L, et al. (2012) Open release of male mosquitoes infected with a Wolbachia biopesticide: field performance and infection containment. PLoS Negl Trop Dis 6(11):e1797.
15. Zhang D, Zheng X, Xi Z, Bourtzis K, & Gilles J R (2015) Combining the sterile insect technique with the incompatible insect technique: I-impact of Wolbachia infection on the fitness of triple- and double-infected strains of Aedes albopictus. PLoS One 10(4):e0121126.
16. Ritchie S A, Townsend M, Paton C J, Callahan A G, & Hoffmann A A (2015) Application of wMelPop Wolbachia strain to crash local populations of Aedes aegypti. PLoS Negl Trop Dis 9(7):e0003930.
17. Zabalou S, et al. (2004) Wolbachia-induced cytoplasmic incompatibility as a means for insect pest population control. Proc Natl Acad Sci U S A 101(42):15042-15045.
18. Hoffmann A A, et al. (2014) Stability of the wMel Wolbachia infection following invasion into Aedes aegypti populations. PLoS Negl Trop Dis 8(9):e3115.
19. Walker T, et al. (2011) The wMel Wolbachia strain blocks dengue and invades caged Aedes aegypti populations. Nature 476(7361):450-453.
20. Dutra H L, et al. (2016) Wolbachia blocks currently circulating Zika virus isolates in Brazilian Aedes aegypti mosquitoes. Cell Host Microbe 19(6):771-774.
21. LePage D P, et al. (2017) Prophage WO genes recapitulate and enhance Wolbachia-induced cytoplasmic incompatibility. Nature 543(7644):243-247.
22. Bordenstein S R & Bordenstein S R (2016) Eukaryotic association module in phage WO genomes from Wolbachia. Nat Commun 7:13155.
23. Beckmann J F, Ronau J A, & Hochstrasser M (2017) A Wolbachia deubiquitylating enzyme induces cytoplasmic incompatibility. Nat Microbiol 2:17007.
24. Gutzwiller F, et al. (2015) Dynamics of Wolbachia pipientis Gene Expression Across the Drosophila melanogaster Life Cycle. G3 (Bethesda) 5(12):2843-2856.
25. Staller M V, et al. (2013) Depleting gene activities in early Drosophila embryos with the “maternal-Gal4-shRNA” system. Genetics 193(1):51-61.
26. Landmann F, Orsi G A, Loppin B, & Sullivan W (2009) Wolbachia-mediated cytoplasmic incompatibility is associated with impaired histone deposition in the male pronucleus. PLoS Pathog 5(3):e1000343.
27. Lassy C W & Karr T L (1996) Cytological analysis of fertilization and early embryonic development in incompatible crosses of Drosophila simulans. Mechanisms of development 57(1):47-58.
28. Callaini G, Riparbelli M G, Giordano R, & Dallai R (1996) Mitotic defects associated with cytoplasmic incompatibility in Drosophila simulans. Journal of Invertebrate Pathology 67(1):55-64.
29. Wright J D & Barr A R (1981) Wolbachia and the normal and incompatible eggs of Aedes polynesiensis (Diptera: Culicidae). Journal of Invertebrate Pathology 38(3):409-418.
30. Duron O & Weill M (2006) Wolbachia infection influences the development of Culex pipiens embryo in incompatible crosses. Heredity (Edinb) 96(6):493-500.
31. Donnelly M L, et al. (2001) Analysis of the aphthovirus 2A/2B polyprotein ‘cleavage’ mechanism indicates not a proteolytic reaction, but a novel translational effect: a putative ribosomal ‘skip’. The Journal of general virology 82(Pt 5):1013-1025.
32. Akhtar W, et al. (2013) Chromatin Position Effects Assayed by Thousands of Reporters Integrated in Parallel. Cell 154(4):914-927.
33. Lindsey A R I, et al. (2018) Evolutionary genetics of cytoplasmic incompatibility genes cifA and cifB in prophage WO of Wolbachia. Genome biology and evolution 10(2):435-451.
34. Brennan L J, Haukedal J A, Earle J C, Keddie B, & Harris H L (2012) Disruption of redox homeostasis leads to oxidative DNA damage in spermatocytes of Wolbachia-infected Drosophila simulans. Insect Mol Biol 21(5):510-520.
35. Penz T, et al. (2012) Comparative genomics suggests an independent origin of cytoplasmic incompatibility in Cardinium hertigii. PLoS Genet 8(10):e1003012.
36. Mann E, et al. (2017) Transcriptome Sequencing Reveals Novel Candidate Genes for Cardinium hertigii-Caused Cytoplasmic Incompatibility and Host-Cell Interaction. mSystems 2(6).
37. Charlat S, Calmet C, & Mercot H (2001) On the mod resc model and the evolution of Wolbachia compatibility types. Genetics 159(4):1415-1422.
38. Bonneau M, et al. (2018) Culex pipiens crossing type diversity is governed by an amplified and polymorphic operon of Wolbachia. Nature Communications 9(1):319.
39. O'Neill S L & Karr T L (1990) bidirectional incompatability between conspecific populations of Drosophila simulans. Nature 348:178-180.
40. Bordenstein S R & Werren J H (2007) Bidirectional incompatibility among divergent Wolbachia and incompatibility level differences among closely related Wolbachia in Nasonia. Heredity 99(3):278-287.
41. Poinsot D, Bourtzis K, Markakis G, Savakis C, & Mercot H (1998) Wolbachia transfer from Drosophila melanogaster into D. simulans: Host effect and cytoplasmic incompatibility relationships. Genetics 150(1):227-237.
42. Bordenstein S R, O'Hara F P, & Werren J H (2001) Wolbachia-induced incompatibility precedes other hybrid incompatibilities in Nasonia. Nature 409(6821):707-710.
43. Brucker R M & Bordenstein S R (2012) Speciation by symbiosis. Trends in ecology & evolution 27(8):443-451.
44. Zabalou S, et al. (2004) Natural Wolbachia infections in the Drosophila yakuba species complex do not induce cytoplasmic incompatibility but fully rescue the wRi modification. Genetics 167(2):827-834.
45. Bourtzis K, Dobson S L, Braig H R, & O'Neill S L (1998) Rescuing Wolbachia have been overlooked. Nature 391(6670):852-853.
46. Casiraghi M, Anderson T J, Bandi C, Bazzocchi C, & Genchi C (2001) A phylogenetic analysis of filarial nematodes: comparison with the phylogeny of Wolbachia endosymbionts. Parasitology 122 Pt 1:93-103.
47. Yu N Y, et al. (2010) PSORTb 3.0: improved protein subcellular localization prediction with refined localization subcategories and predictive capabilities for all prokaryotes. Bioinformatics (Oxford, England) 26(13):1608-1615.
48. Hofmann K (1993) TMbase-A database of membrane spanning proteins segments. Biol. Chem. Hoppe-Seyler 374:166.
49. Kumar S, Stecher G, & Tamura K (2016) MEGA7: Molecular Evolutionary Genetics Analysis Version 7.0 for Bigger Datasets. Molecular biology and evolution 33(7):1870-1874.
50. Liang H, Zhou W, & Landweber L F (2006) SWAKK: a web server for detecting positive selection in proteins using a sliding window substitution rate analysis. Nucleic Acids Res 34(Web Server issue):W382-384.
51. Steinway S N, Dannenfelser R, Laucius C D, Hayes J E, & Nayak S (2010) JCoDA: a tool for detecting evolutionary selection. BMC Bioinformatics 11:284.

### Example 2. Synthetic Recapitulation by Transgenic Expression of cifA and cifB in Drosophila

Wolbachia are maternally inherited bacteria that infect many arthropod species and are deployed in vector control to curb arboviral spread using cytoplasmic incompatibility (CI). CI kills offspring when an infected male mates an uninfected female, but the lethality is rescued if the female is likewise infected. Two phage genes, cifA_{wMel} and cifB_{wMel} from wMel Wolbachia deployed in vector control, transgenically recapitulate variably penetrant CI, and one of the same genes, cifA_{wMel}, rescues wild type CI. Resultantly, the Two-by-One model of CI predicts that CI and rescue can be recapitulated by transgenic expression alone and that dual cifA_{wMel} and cifB_{wMel} expression can recapitulate strong CI. It is shown here that CI and rescue can be synthetically recapitulated in full, and transgenic CI comparable to wild type CI is achievable. These data validate the Two-by-One model, establish methods for transgenic studies of CI, and represent the first case of completely engineering male and female animal reproduction to depend upon bacteriophage gene products.

The World Health Organization recently recommended deployment of Wolbachia-infected mosquitoes for pilot biocontrol efforts that curb the transmission of Zika and dengue viruses to humans. These releases are underway worldwide because Wolbachia block replication of these pathogenic viruses and spread themselves maternally through arthropod populations via cytoplasmic incompatibility (CI). The CI drive system depends on a Wolbachia-induced sperm modification that results in embryonic lethality when an infected male mates with an uninfected female, but this lethality is rescued when the female and her eggs are likewise infected. Two separate studies reported that the phage WO genes, cifA and cifB, cause the sperm modification and cifA rescues the embryonic lethality caused by the wMel Wolbachia strain deployed in vector control. The example herein shows explicit support for the Two-by-One model of CI model whereby two genes cause lethality and one gene rescues it, using synthetic methods that recapitulate CI and rescue in the absence of Wolbachia infections. Notably, these results constitute the first case of engineering animal reproduction to be entirely dependent on phage genes.

### Background

Wolbachia are the most widespread endosymbiotic bacteria on the planet and are estimated to infect half of all arthropod species and half of the Onchocercidae family of filarial nematodes. They specialize in infecting the cells of reproductive tissues, are primarily inherited maternally from ova to offspring, and often act in arthropods as reproductive parasites that enhance their maternal transmission by distorting host sex ratios and reproduction. The most common type of reproductive parasitism is cytoplasmic incompatibility (CI), which manifests as a sperm modification in infected males that causes embryonic lethality or haploidization in matings with uninfected females upon fertilization. This embryonic lethality is rescued if the female is infected with the same Wolbachia strain. As such, CI selfishly drives CI-inducing Wolbachia into host populations, and the incompatibilities between host populations cause reproductive isolation between recently diverged or incipient species.

In the last decade, Wolbachia and CI have garnered significant interest for their utility in combatting vector borne diseases worldwide. Two strategies are currently deployed: population suppression and population replacement. The population suppression strategy markedly crashes vector population sizes through the release of only infected males that induce CI upon mating with wild uninfected females. In contrast, the population replacement strategy converts uninfected to infected populations through the release of both infected males and females that aid the spread Wolbachia via CI and rescue. Replacing a vector competent, uninfected population with infected individuals can notably reduce the spread of arthropod borne diseases such as Zika and dengue because Wolbachia appear to inhibit various stages of viral replication within arthropods based on diverse manipulations of the host cellular environment. The combination of Wolbachia's abilities to suppress arthropod populations, drive into host populations, and block the spread of viral pathogens have established Wolbachia in the vanguard of vector control efforts to curb arboviral transmission.

An unbiased, multi-omic analysis of CI-inducing and CI-incapable Wolbachia strains revealed two adjacent genes, cifA and cifB, in the eukaryotic association module of prophage WO that strictly associate with CI induction. Fragments of the CifA protein were found in the fertilized spermathecae of infected Culex pipiens mosquitoes, and these genes are frequently missing or degraded in diverse CI-incapable strains. Dual transgenic expression of cifA and cifB from either of the CI inducing strains wMel or wPip in uninfected male flies causes a decrease in embryonic hatching corresponding to an increase in CI-associated cytological abnormalities including chromatin bridging and regional mitotic failures. Single transgenic expression of either cifA_{wMel} or cifB_{wMel} in an uninfected male was insufficient to recapitulate CI, but single transgenic expression of either gene in an infected male can enhance wMel-induced CI in a dose-dependent manner To establish the lethality as CI, transgenic CI induced by cifA_{wMel} and cifB_{wMel} expressing males was rescued when they were mated with wMel-infected females. Transgenic expression of cifA_{wMel} alone in uninfected females also rescues embryonic lethality and nullifies cytological defects associated with wild type CI caused by a wMel infection. These data show the Two-by-One genetic model of CI wherein dual expression of cifA_{wMel} and cifB_{wMel} causes CI when expressed in males and expression of cifA_{wMel} rescues CI when expressed in females. However, confirmation of the model's central prediction requires the complete synthetic replication of CI-induced lethality and rescue in the absence of any Wolbachia infections. Moreover, CI induced by dual cifA_{wMel} and cifB_{wMel} expression previously yielded variable CI-like lethality with a median survival of 26.5% of embryos relative to survival of 0.0% of embryos from CI induced by a wild type infection. The inability to recapitulate strong wild type CI shows other CI genes are required, environmental factors need to be controlled, or the transgenic system requires additional improvements.

In this example, transgenic expression, hatch rates, and gene expression assays in Drosophila melanogaster are utilized to test if an improved expression system can generate strong transgenic CI and whether these multi-domain bacteriophage genes, cifA_{wMel} and cifB_{wMel}, can fully control fly reproduction by inducing and rescuing CI in the complete absence of Wolbachia (FIG. 11). Furthermore, it is shown that both cif_{wMel} genes are required for CI induction in the improved system and that cifA_{wMel} in females can rescue transgenic CI. Results provide strong evidence for the Two-by-One model in wMel, offer context for conceptualizing the evolution of bidirectional incompatibilities between different Wolbachia strains, raise points for CI gene nomenclature, and provide tools to combat vector borne diseases. These data also represent the first case of completely engineering animal sexual reproduction to depend upon bacteriophage gene products.

### Results and Discussion

Investigation of transgenic CI expression: Dual transgenic expression of cifA_{wMel} and cifB_{wMel} was previously reported to induce highly variable and incomplete CI relative to CI caused by a wild type infection, indicating either the presence of other genes required for strong CI, environmental factors uncontrolled in the study, or inefficiency in the transgenic system. Here, the inefficiency in the transgenic system is tested by co-expressing cifA_{wMel} and cifB_{wMel} in D. melanogaster males under two GAL4 drivers that express in reproductive tissues: nos-GAL4-tubulin and nos-GAL4:VP16. Both drivers contain a nos promoter region, but differ in that nos-GAL4-tubulin produces a transcription factor with both the DNA binding and transcriptional activating region of the GAL4 protein, and nos-GAL4:VP16 produces a fusion protein of the GAL4 DNA binding domain and the virion protein 16 (VP16) activating region. The GAL4:VP16 transcription factor is a particularly potent transcriptional activator because of its binding efficiency to transcription factors. Additionally, the nos-GAL4-tubulin driver has a tubulin 3′ UTR, and nos-GAL4:VP16 has a nos 3′ UTR that contribute to differences in localization.

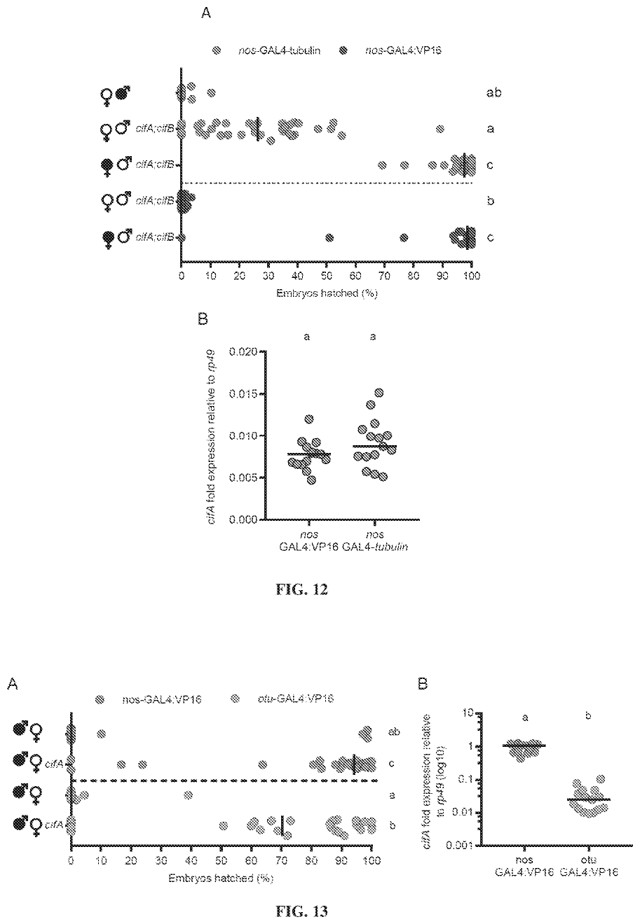

Since CI manifests as embryonic lethality, the hatching of D. melanogaster embryos into larvae is measured to quantify the strength of CI. Transgenic expression of both cifA_{wMel} and cifB_{wMel} under nos-GAL4-tubulin in uninfected males yields low but variable embryonic hatching in crosses with uninfected females (Mdn=26.3%, IQR=10.4-38.1%) that can in turn be rescued in crosses with wMel-infected females (Mdn=97.5%; IQR=94.2-100%) (FIG. 12A). However, dual expression under nos-GAL4:VP16 in uninfected males yields significantly reduced embryonic hatching relative to nos-GAL4-tubulin (p=0.0002) with less variability (Mdn=0%; IQR=0.0-0.75%) and can be comparably rescued (Mdn=98.65%; IQR=95.93-100%; p>0.99) (FIG. 12A). Together, these results support that dual cifA_{wMel} and cifB_{wMel} expression under nos-GAL4:VP16 induces the strongest CI and supports that the transgenic system, not the absence of necessary CI factors, contributed to the prior inability to recapitulate strong wild type CI.

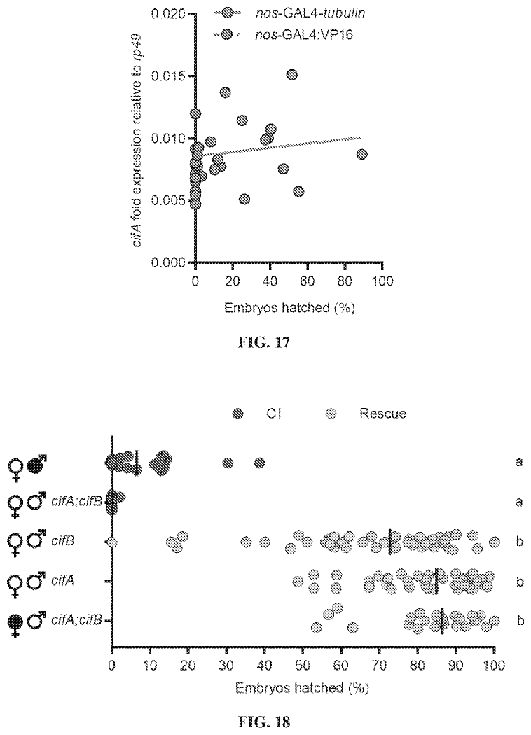

The next experiment tests whether differences in the penetrance of transgenic CI between the two drivers are due to differences in the strength of transgenic expression. To assess this, qPCR was used to measure the gene expression of cifA_{wMel} under the two drivers relative to a Drosophila housekeeping gene (rp49) in male abdomens (FIG. 12B). Fold RNA transcript differences of cifA_{wMel} relative to rp49 reveal nos-GAL4-tubulin causes higher and more variable expression (Mdn=0.0087; IQR=0.0075-0.0108) than nos-GAL4:VP16 (Mdn=0.0078; IQR=0.0066-0.0086), but these differences are statistically insignificant (p=0.16). Moreover, the level of cifA_{wMel} expression under the more variable nos-GAL4-tubulin driver does not correlate with the strength of CI measured via hatch rates (R²=0.02; p=0.62; FIG. 17). These results show that variation in phenotypic penetrance in these crosses is not associated with variation in transgene transcript abundance from different drivers.

Investigation of transgenic rescue expression: The maternal triple driver (MTD) can rescue CI induced by a wild type infection when expressing cifA_{wMel} in uninfected females. It is comprised of three drivers: nos-GAL4-tubulin, nos-GAL4:VP16, and otu-GAL4:VP16. The nos-GAL4-tubulin driver has previously been reported to be rescue-incapable. Here, it is shown that either of the other components of the MTD driver independently recapitulate rescue of wMel CI. Hatch rate experiments indicate that CI is strong and expectedly not rescued when an infected male mates with a non-transgenic female whose genotype is otherwise nos-GAL4:VP16 (Mdn=0.0%; IQR=0.0-0.0%) or otu-GAL4:VP16 (Mdn=0.0%; IQR=0.0-0.0%) (FIG. 13A). Transgenic expression of cifA_{wMel} in uninfected females under either of the two drivers rescues CI induced by wMel. However, rescue is significantly weaker under the otu-GAL4:VP16 driver (Mdn=70.4%; IQR=0.0-90.45%) as compared to the nos-GAL4:VP16 driver (Mdn=94.2%; IQR=83.3-97.1%; p=0.0491) which produced strong transgenic rescue (FIG. 13A). Gene expression analysis of cifA_{wMel} relative to rp49 in the abdomens of uninfected females reveals that nos-GAL4:VP16 expresses cifA_{wMel} significantly higher (Mdn=1.08; p<0.0001) than otu-GAL4:VP16 (Mdn=0.03) (FIG. 13B), showing that high expression in females underpins the ability to rescue. Alternatively, nos-GAL4:VP16 and otu-GAL4:VP16 are known to express at different times in oogenesis, with the former in all egg chambers and the latter in late stage egg chambers.

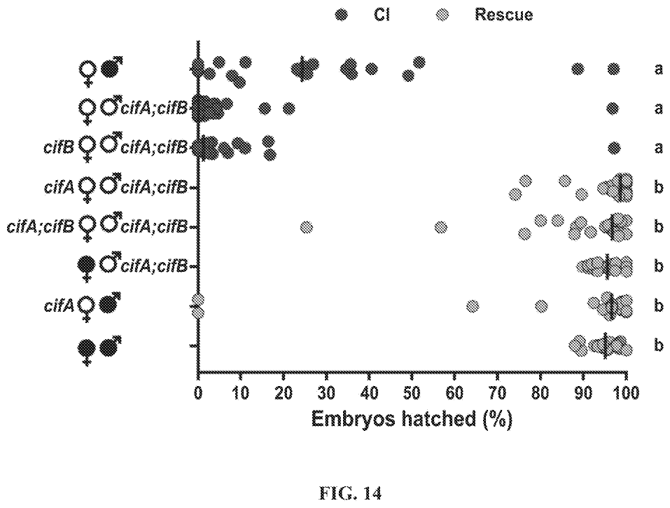

The Two-by-One model of CI: With the transgenic expression system improved for both transgenic CI and rescue, it is shown herein that the Two-by-One model of CI can be synthetically recapitulated by dual cifA_{wMel} and cifB_{wMel} expression in uninfected males to cause CI, and single cifA_{wMel} expression in uninfected females to rescue that transgenic CI. Indeed, dual cifA_{wMel} and cifB_{wMel} expression in males causes hatch rates comparable to wild type CI (Mdn=0.0%; IQR=0.0%-2.55; p>0.99) (FIG. 14). Transgenic CI cannot be rescued by single cifB_{wMel} expression in females (Mdn=1.25%; IQR=0.0-3.35%). Transgenic CI can be rescued by single cifA_{wMel} expression (Mdn=98.6%; IQR=97.35-100%; p=0.41) or dual cifA_{wMel} and cifB_{wMel} expression (Mdn=96.7%; IQR=88.3-98.2%; p>0.99) to levels comparable to rescue from a wild type infection (Mdn=95.6%; IQR=92.5-97.4%). In addition, cifA_{wMel} rescues a wild type infection at comparable levels to wild type rescue (Mdn=96.6%; IQR=93.5-98.85%; p>0.99). These data provide strong evidence for the Two-by-One model, namely that CI induced by transgenic dual cifA_{wMel} and cifB_{wMel} expression is sufficient to induce strong CI and that cifA_{wMel} alone is sufficient to rescue it.

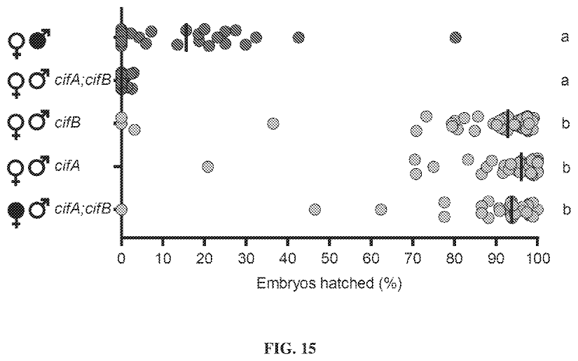

The next experiment reevaluated if single cifA_{wMel} or cifB_{wMel} expression under the more potent nos-GAL4:VP16 driver in uninfected males can recapitulate CI. Hatch rates indicate that dual cifA_{wMel} and cifB_{wMel} expression induces strong transgenic CI (Mdn=0.0%; IQR=0.0-1.15%) that can be rescued by a wild type infection (Mdn=93.8%; IQR=88.2-97.4%), whereas single expression of cifA_{wMel} (Mdn=96.1%; IQR=97.78-98.55%; p<0.0001) or cifB_{wMel} (Mdn=92.85%; IQR=84.28-96.4%; p<0.0001) failed to produce embryonic hatching comparable to expressing both genes together (FIG. 15). In one replicate experiment, a statistically insignificant (p=0.182) decrease is noted in hatching under cifB_{wMel} expression relative to wild type rescue cross (FIG. 18). Thus, both cifA_{wMel} and cifB_{wMel} are required for strong CI. Together, these and earlier results validate the Two-by-One model of CI in wMel where cifA_{wMel} and cifB_{wMel} expression are required and sufficient for strong CI, while cifA_{wMel} expression is sufficient to rescue it.

CI is the most common form of Wolbachia-induced reproductive parasitism and is currently at the forefront of vector control efforts to curb transmission of dengue, Zika, and other arthropod borne human pathogens. Two prophage WO genes from wMel Wolbachia cause CI (cifA_{wMel} and cifB_{wMel}) and one rescues wild type CI (cifA_{wMel}), supporting the proposal of a Two-by-One model for the genetic basis of CI. In addition, the Two-by-One model predicts that both CI and rescue can be synthetically recapitulated by dual cifA_{wMel} and cifB_{wMel} expression in uninfected males and cifA_{wMel} expression in uninfected females. The work shown here improves the transgenic system for CI and rescue by these genes, further validated the necessity of expressing both cifA_{wMel} and cifB_{wMel} for CI, and synthetically recapitulated the Two-by-One model for CI with transgenics in the absence of Wolbachia.

CI induced by wMel Wolbachia can be highly variable and correlates with numerous factors including Wolbachia density, cifA_{wMel} and cifB_{wMel} expression levels, host age, mating rate, rearing density, and development time. Some of these factors, such as age, are known to also correlate with the level of cif_{wMel} gene expression. As such, the weakened transgenic CI can be explained by low levels of transgenic cifA_{wMel} and cifB_{wMel} expression in male testes.

Indeed, strong CI with a median of 0% embryonic hatching was induced when both cifA_{wMel} and cifB_{wMel} were expressed under the nos-GAL4:VP16 driver. However, nos-GAL4:VP16 did not generate significantly higher cifA_{wMel} expression than the nos-GAL4-tubulin driver previously used to recapitulate weak CI. Thus, the expression data conflict with previous reports in mammalian cells wherein the GAL4:VP16 fusion protein is surprisingly a more potent transcriptional activator than GAL4. Other differences between the two driver constructs may explain phenotypic differences, including the presence of different 3′ UTRs that may contribute to differences in transcript localization. In addition, the induction of near complete embryonic lethality confirms that cifA_{wMel} and cifB_{wMel} are sufficient to induce strong CI and do not require other genes to do so. Moreover, comparative multi-omics demonstrated that cifA and cifB are the only two genes strictly associated with CI capability.

Rescue of CI induced by a wild type wMel-infection was previously recapitulated through expression of cifA_{wMel} under the Maternal Triple Driver (MTD), which is comprised of three independent drivers. While one of the MTD drivers was previously shown not to be rescue capable, neither of the other drivers were tested. Here, it is shown that one of the remaining drivers is sufficient to induce rescue when expressing cifA_{wMel} and that both drivers induce a phenotype, but at different strengths. In contrast to induction of transgenic CI wherein improved induction efficiency was not dependent on RNA expression changes, the transgenic driver inducing the highest expression also generated the strongest rescue. These data are consistent with reports that cifA_{wMel} is a highly expressed gene in transcriptomes of wMel-infected females and proving that rescue capability is largely determined by the strength of cifA_{wMel} expression in ovaries.

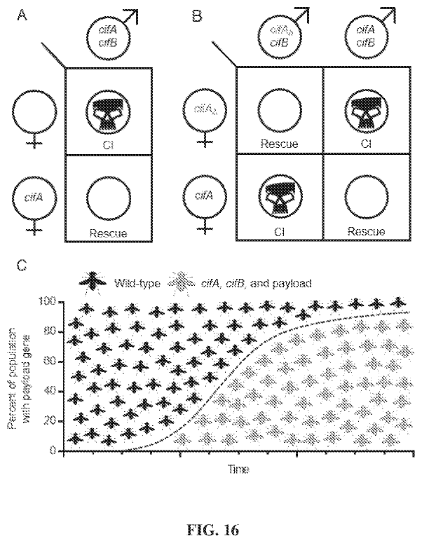

The central prediction of the Two-by-One model is that both CI and rescue can be synthetically recapitulated in the absence of Wolbachia through dual cifA_{wMel} and cifB_{wMel} expression in uninfected males and cifA_{wMel} expression in uninfected females. Here, it is shown that dual expression in males is sufficient to induce strong CI and that cifA_{wMel} alone is sufficient to rescue transgenic CI. Thus, these data strongly support the model that two genes are required in males to cause CI, and one in females is required to rescue it. However, to confirm that the improved expression system does not influence the ability of cifA_{wMel} or cifB_{wMel} alone to induce CI, these genes are singly expressed with the improved driver, showing that embryonic hatching does not statistically differ from compatible crosses. These results validate the Two-by-One genetic model whereby cifA_{wMel} and cifB_{wMel} are both required in the testes to cause embryonic death that can be rescued by cifA_{wMel} in the ovaries (FIG. 16A).

It has been shown that divergence in CI and rescue factors causes the incipient evolution of reciprocal incompatibility, or bidirectional CI, between different Wolbachia strains. Here, the data explains the emergence of bidirectional CI consistent with the Two-by-One model. First, the simplest explanation for cifA's role in both CI and rescue is that it has similar functional effects in both testes and ovaries. Thus, instead of requiring a separate mutation for CI and another for rescue bidirectional CI emerges from a single CifA mutation that causes incompatibility against the ancestral strain while maintaining self-compatibility (FIG. 16B). Second, CifA in testes and ovaries have different functions, localizations, or posttranslational modifications that contribute to CI and rescue. If this occurs, or if CifB is also an incompatibility factor, the evolution of bidirectional CI requires two or more mutations, and the strain passes through an intermediate phenotype wherein it becomes unidirectionally incompatible with the ancestral variant or loses the capability to induce either CI or rescue before becoming bidirectionally incompatible with the ancestral variant. In fact, some Wolbachia strains are incapable of inducing CI but capable of rescuing CI induced by other strains, and some can induce CI but cannot be rescued. Furthermore, sequence variation in both cifA and cifB from Wolbachia strains in Drosophila and in small regions among strains of wPip Wolbachia have been correlated to incompatibility, indicating that variation in both genes influence incompatibility.

Additionally, significant divergence in cifA, cifB, or both is necessary to generate new phenotypes. Indeed, comparative genomic analyses reveal high levels of amino acid divergence in CifA and CifB that correlates with incompatibility between strains. Moreover, some Wolbachia strains harbor numerous phage WO variants, each with their own, often divergent, cif genes, and the presence of multiple variants likewise correlates with incompatibility. Thus, horizontal transfer of phage WO can in theory rapidly introduce new compatibility relationships, and duplication of phage WO regions, or specifically cif genes, in the same Wolbachia genome relax the selective pressure on the cif genes and enable their divergence. Determining which of the aforementioned models best explains the evolution of incompatibilities between Wolbachia strains is assisted by additional sequencing studies to identify incompatible strains with closely related cif variants.

The genetic bases of numerous gene drives have been elucidated in plants, fungi, and nematodes. Some gene drives have also been artificially replicated with transgenic constructs. However, the synthetic replication of the Two-by-One model of CI represents the first instance that a gene drive has been constructed by engineering eukaryotic reproduction to depend on phage proteins. Additionally, vector control programs using Wolbachia rely on their ability to suppress pathogens such as Zika and dengue viruses, reduce the size of vector populations, and spread Wolbachia into a host population via CI and rescue. However, there are limitations to these approaches. Most critically, not all pathogens are inhibited by Wolbachia infection and some are enhanced, such as malaria in Anopheles gambiae and West Nile Virus in Culex tarsalis, which are both infected with wAlbB Wolbachia. The synthetic replication of CI and rescue via the Two-by-One model represents a step towards using the cif genes in vector control efforts separate from Wolbachia. The separation of CI mechanism from Wolbachia infection expands CI's utility to spread vector suppressing ‘payload’ genes into a host population (FIG. 16C) by, for instance, expressing the CI genes and the payload gene polycistronically under the same promoter in the vector nuclear or mitochondrial genomes. Moreover, these synthetic constructs can increase the efficiency of Wolbachia-induced CI if they are transformed directly into Wolbachia genomes.

Finally, these results further show the importance of cifA_{wMel} as an essential component of CI and underscore a community need to unify the nomenclature of the CI genes. When the CI genes were first reported, they were described as both CI factors (cif) and as CI deubiquitilases (cid), both of which are actively utilized in the literature. The cif nomenclature was proposed as a conservative naming strategy agnostic to biochemical function, whereas the cid nomenclature was proposed based on the finding that the B protein is at least a deubiquitilase that, when ablated, inhibits CI induction. However, CifA is not a putative deubiquitilase, does not influence deubiquitilase activity of CifB, functions independently to rescue CI and, as emphasized by the work in this study, is necessary for CI induction and rescue. Thus, the holistic and conservative cif nomenclature is appropriately warranted in utilizing and unifying CI gene names.

In conclusion, the results presented here support that both cifA_{wMel} and cifB_{wMel} phage genes are necessary and sufficient to induce strong CI. In addition, cifA_{wMel} is the only gene necessary for rescue of either transgenic or wild type wMel CI. These results confirm the Two-by-One model of CI in wMel Wolbachia and phage WO with indication for the diversity of incompatibility between strains, and they provide additional context for understanding CI currently deployed in vector control efforts. Finally, the synthetic replication of CI in the absence of Wolbachia provides a tool for genetic and mechanistic studies in D. melanogaster and for vector control efforts that can drive payload genes into vector competent populations.

### Materials and Methods

Fly rearing and strains. D. melanogaster stocks y¹w* (BDSC 1495), nos-GAL4-tubulin (BDSC 4442), nos-GAL4:VP16 (BDSC 4937), otu-GAL4:VP16 (BDSC 58424), and UAS transgenic lines homozygous for cifA, cifB, and cifA; B (39) were maintained at 12:12 light:dark at 25° C. and 70% relative humidity (RH) on 50 ml of a standard media. UAS transgenic lines and nos-GAL4:VP16 were uninfected whereas nos-GAL4-tubulin and otu-GAL4:VP16 lines were infected with wMel Wolbachia. Uninfected versions of infected lines were produced through tetracycline treatment as previously described. WolbF and WolbR3 primers were regularly used to confirm infection status. Stocks for virgin collections were stored at 18° C. overnight to slow eclosion rate, and virgin flies were kept at room temperature.

Hatch rate assays. To test for CI, hatch rate assays were used as previously described. Briefly, GAL4 adult females were aged 9-11 days post eclosion and mated with UAS males. Age controlled GAL4-UAS males and females were paired in 8 oz bottles affixed with a grape-juice agar plate smeared with yeast affixed to the opening with tape. The flies and bottles were stored at 25° C. for 24 h at which time the plates were replaced with freshly smeared plates and again stored for 24 h. Plates were then removed and the number of embryos on each plate were counted and stored at 25° C. After 30 h the remaining unhatched embryos were counted. The percent of embryos hatched into larvae was calculated by dividing the number of hatched embryos by the initial embryo count and multiplying by 100.

Expression analyses. To assay transgenic RNA expression levels under the various gene drive systems, transgene expressing flies from hatch rates were immediately collected and frozen at −80° C. for downstream application as previously described. In brief, abdomens were dissected, RNA was extracted using the Direct-zol RNA MiniPrep Kit (Zymo), the DNA-free kit (Ambion, Life Technologies) was then used to remove DNA contamination, and cDNA was generated with SuperScript VILO (Invitrogen). Quantitative PCR was performed on a Bio-Rad CFX-96 Real-Time System using iTaq Universal SYBR Green Supermix (Bio-Rad) using the cifA_opt and rp49 forward and reverse primers as previously described (44). Fold expression of cifA relative to rp49 was determined with 2^{-ΔΔCt}.

Statistical analyses. All statistical analyses were conducted in GraphPad Prism (Prism 8). Hatch rate statistical comparisons were made using Kruskal-Wallis followed by a Dunn's multiple comparison test. A Mann-Whitney-U was used for statistical comparison of RNA fold expression. A linear regression was used to assess correlations between hatch rate and expression.

### REFERENCES CITED IN THIS EXAMPLE

1. Weinert L A, Araujo-Jnr E V, Ahmed M Z, Welch J J. The incidence of bacterial endosymbionts in terrestrial arthropods. Proc R Soc B. 2015 May. 22; 2282(1807):20150249.
2. Zug R, Hammerstein P. Still a Host of Hosts for Wolbachia: Analysis of Recent Data Suggests That 40% of Terrestrial Arthropod Species Are Infected. PLOS ONE. 2012 Jun. 7; 7(6):e38544.
3. Ferri E, Bain O, Barbuto M, Martin C, Lo N, Uni S, et al. New Insights into the Evolution of Wolbachia Infections in Filarial Nematodes Inferred from a Large Range of Screened Species. PLOS ONE. 2011 Jun. 22; 6(6):e20843.
4. LePage D, Bordenstein S R. Wolbachia: Can we save lives with a great pandemic? Trends in Parasitology. 2013 Aug. 1; 29(8):385-93.
5. Taylor M J, Bordenstein S R, Slatko B. Microbe Profile: Wolbachia: a sex selector, a viral protector and a target to treat filarial nematodes. Microbiology. 2018 Nov. 1; 164(11):1345-7.
6. Werren J H, Baldo L, Clark M E. Wolbachia: master manipulators of invertebrate biology. Nature Reviews Microbiology. 2008 October; 6(10):741-51.
7. Bordenstein S R, Uy J J, Werren J H. Host genotype determines cytoplasmic incompatibility type in the haplodiploid genus <i>Nasonia<i>. Genetics. 2003 May; 164(1):223-33.
8. Serbus L R, Casper-Lindley C, Landmann F, Sullivan W. The Genetics and Cell Biology of Wolbachia-Host Interactions. Annual Review of Genetics. 2008; 42(1):683-707.
9. Yen J H, Barr A R. The etiological agent of cytoplasmic incompatibility in Culex pipiens. Journal of Invertebrate Pathology. 1973 Sep. 1; 22(2):242-50.
10. Hancock P A, Sinkins S P, Godfray H C J. Population Dynamic Models of the Spread of Wolbachia. The American Naturalist. 2011 Mar. 1; 177(3):323-33.
11. Hoffmann A, Turelli M, Harshman L. Factors Affecting The Distribution of Cytoplasmic Incompatibility in Drosophila simulans. Genetics. 1990 December; 126(4):933-48.
12. Leftwich P T, Edgington M P, Harvey-Samuel T, Carabajal Paladino L Z, Norman V C, Alphey L. Recent advances in threshold-dependent gene drives for mosquitoes. Biochem Soc Trans. 2018 Oct. 19; 46(5):1203-12.
13. Turelli M. Evolution of Incompatibility-Inducing Microbes and Their Hosts. Evolution. 1994 October; 48(5):1500-13.
14. Turelli M, Cooper B S, Richardson K M, Ginsberg P S, Peckenpaugh B, Antelope C X, et al. Rapid Global Spread of wRi-like Wolbachia across Multiple Drosophila. Current Biology. 2018 Mar. 19; 28(6):963-971.e8.
15. Bordenstein S R, O'Hara F P, Werren J H. Wolbachia-induced incompatibility precedes other hybrid incompatibilities in Nasonia. Nature. 2001 Feb. 8; 409(6821):707-10.
16. Brucker R M, Bordenstein S R. Speciation by symbiosis. Trends in Ecology & Evolution. 2012 Aug. 1; 27(8):443-51.
17. Jaenike J, Dyer KA, Cornish C, Minhas M S. Asymmetrical reinforcement and Wolbachia infection in Drosophila. PLoS Biol. 2006 October; 4(10):e325.
18. Miller W J, Ehrman L, Schneider D. Infectious speciation revisited: impact of symbiont-depletion on female fitness and mating behavior of Drosophila paulistorum. PLoS Pathog. 2010 Dec. 2; 6(12):e1001214.
19. Shropshire J D, Bordenstein S R. Speciation by Symbiosis: the Microbiome and Behavior. mBio. 2016 May 4; 7(2):e01785-15.
20. Dobson S L, Fox Charles W., Jiggins Francis M. The effect of Wolbachia-induced cytoplasmic incompatibility on host population size in natural and manipulated systems. Proceedings of the Royal Society of London Series B: Biological Sciences. 2002 Mar. 7; 269(1490):437-45.
21. Lees R S, Gilles J R, Hendrichs J, Vreysen M J, Bourtzis K. Back to the future: the sterile insect technique against mosquito disease vectors. Curr Opin Insect Sci. 2015 August; 10: 156-62.
22. Nikolouli K, Colinet H, Renault D, Enriquez T, Mouton L, Gibert P, et al. Sterile insect technique and Wolbachia symbiosis as potential tools for the control of the invasive species Drosophila suzukii. Journal of Pest Science. 2018 March; 91(2):489-503.
23. O'Connor L, Plichart C, Sang A C, Brelsfoard C L, Bossin H C, Dobson S L. Open release of male mosquitoes infected with a Wolbachia biopesticide: field performance and infection containment. PLoS Negl Trop Dis. 2012; 6(11):e1797.
24. Huang M, Luo J, Hu L, Zheng B, Yu J. Assessing the efficiency of Wolbachia driven Aedes mosquito suppression by delay differential equations. J Theor Biol. 2018 Mar. 7; 440:1-11.
25. O'Neill S L. The Use of Wolbachia by the World Mosquito Program to Interrupt Transmission of Aedes aegypti Transmitted Viruses. Adv Exp Med Biol. 2018; 1062:355-60.
26. Caragata E P, Dutra H L C, Moreira L A. Inhibition of Zika virus by Wolbachia in Aedes aegypti. Microb Cell. 2016; 3(7):293-5.
27. Hoffmann A A, Montgomery B L, Popovici J, Iturbe-Ormaetxe I, Johnson P H, Muzzi F, et al. Successful establishment of Wolbachia in Aedes populations to suppress dengue transmission. Nature. 2011 August ; 476(7361):454-7.
28. Bhattacharya T, Newton I L G, Hardy R W. Wolbachia elevates host methyltransferase expression to block an RNA virus early during infection. PLOS Pathogens. 2017 Jun. 15; 13(6):e1006427.
29. Brennan L J, Haukedal J A, Earle J C, Keddie B, Harris H L. Disruption of redox homeostasis leads to oxidative DNA damage in spermatocytes of Wolbachia-infected Drosophila simulans. Insect Mol Biol. 2012 October; 21(5):510-20.
30. Caragata E P, Rancès E, Hedges L M, Gofton A W, Johnson K N, O'Neill S L, et al. Dietary cholesterol modulates pathogen blocking by Wolbachia. PLoS Pathog. 2013; 9(6):e1003459.
31. Geoghegan V, Stainton K, Rainey S M, Ant T H, Dowle A A, Larson T, et al. Perturbed cholesterol and vesicular trafficking associated with dengue blocking in Wolbachia-infected Aedes aegypti cells. Nat Commun. 2017 13; 8(1):526.
32. Lindsey A, Bhattacharya T, Newton I, Hardy R. Conflict in the Intracellular Lives of Endosymbionts and Viruses: A Mechanistic Look at Wolbachia-Mediated Pathogen-blocking. Viruses. 2018 Mar. 21; 10(4):141.
33. Molloy J C, Sommer U, Viant M R, Sinkins S P. Wolbachia Modulates Lipid Metabolism in Aedes albopictus Mosquito Cells. Appl Environ Microbiol. 2016 15; 82(10):3109-20.
34. Schultz M J, Isern S, Michael S F, Corley R B, Connor J H, Frydman H M. Variable Inhibition of Zika Virus Replication by Different Wolbachia Strains in Mosquito Cell Cultures. J Virol. 2017 15; 91(14).
35. Hughes G L, Koga R, Xue P, Fukatsu T, Rasgon J L. Wolbachia Infections Are Virulent and Inhibit the Human Malaria Parasite Plasmodium Falciparum in Anopheles Gambiae. PLOS Pathogens. 2011 May 19; 7(5):e1002043.
36. Schmidt T L, Barton N H, Rašić G, Turley A P, Montgomery B L, Iturbe-Ormaetxe I, et al. Local introduction and heterogeneous spatial spread of dengue-suppressing Wolbachia through an urban population of Aedes aegypti. PLOS Biology. 2017 May 30; 15(5):e2001894.
37. Turelli M, Barton N H. Deploying dengue-suppressing Wolbachia: Robust models predict slow but effective spatial spread in Aedes aegypti. Theor Popul Biol. 2017; 115:45-60.
38. Bordenstein S R, Bordenstein S R. Eukaryotic association module in phage WO genomes from Wolbachia. Nature Communications. 2016 Oct. 11; 7:13155.
39. LePage D P, Metcalf J A, Bordenstein S R, On J, Perlmutter J I, Shropshire J D, et al. Prophage WO genes recapitulate and enhance Wolbachia-induced cytoplasmic incompatibility. Nature. 2017 Feb. 27; 543(7644):243-7.
40. Beckmann J F, Fallon A M. Detection of the Wolbachia protein WPIP0282 in mosquito spermathecae: implications for cytoplasmic incompatibility. Insect Biochem Mol Biol. 2013 September; 43(9):867-78.
41. Lindsey A, Rice D W, Bordenstein S R, Brooks A W, Bordenstein S R, Newton I L G. Evolutionary Genetics of Cytoplasmic Incompatibility Genes cifA and cifB in Prophage WO of Wolbachia. Genome Biol Evol. 2018 Feb. 1; 10(2):434-51.
42. Sutton E R, Harris S R, Parkhill J, Sinkins S P. Comparative genome analysis of Wolbachia strain wAu. BMC Genomics. 2014 Oct. 24; 15(1):928.
43. Beckmann J F, Ronau J A, Hochstrasser M. A Wolbachia deubiquitylating enzyme induces cytoplasmic incompatibility. Nature Microbiology. 2017 May; 2(5):17007.
44. Shropshire J D, On J, Layton E M, Zhou H, Bordenstein S R. One prophage WO gene rescues cytoplasmic incompatibility in Drosophila melanogaster. Proceedings of the National Academy of Sciences. 2018 Apr. 23; 201800650.
45. Ni J-Q, Zhou R, Czech B, Liu L-P, Holderbaum L, Yang-Zhou D, et al. A genome-scale shRNA resource for transgenic RNAi in Drosophila. Nature Methods. 2011 May; 8(5):405-7.
46. Doren M V, Williamson A L, Lehmann R Regulation of zygotic gene expression in Drosophila primordial germ cells. Current Biology. 1998 Feb. 12; 8(4):243-6.
47. Tracey W D, Ning X, Klingler M, Kramer S G, Gergen J P. Quantitative analysis of gene function in the Drosophila embryo. Genetics. 2000 January; 154(1):273-84.
48. He Z, Brinton B T, Greenblatt J, Hassell J A, Ingles C J. The transactivator proteins VP16 and GAL4 bind replication factor A. Cell. 1993 Jun. 18; 73(6):1223-32.
49. Sadowski I, Ma J, Triezenberg S, Ptashne M. GAL4-VP16 is an unusually potent transcriptional activator. Nature. 1988 October; 335(6190):563-4.
50. Bourtzis K, Nirgianaki A, Markakis G, Savakis C Wolbachia infection and cytoplasmic incompatibility in Drosophila species. Genetics. 1996 November; 144(3):1063-73.
51. Awrahman Z A, Champion de Crespigny F, Wedell N. The impact of Wolbachia, male age and mating history on cytoplasmic incompatibility and sperm transfer in Drosophila simulans. J Evol Biol. 2014 January; 27(1):1-10.
52. Reynolds K T, Thomson L J, Hoffmann A A. The effects of host age, host nuclear background and temperature on phenotypic effects of the virulent Wolbachia strain popcorn in Drosophila melanogaster. Genetics. 2003 July; 164(3):1027-34.
53. Reynolds K T, Hoffmann A A. Male age, host effects and the weak expression or non-expression of cytoplasmic incompatibility in Drosophila strains infected by maternally transmitted Wolbachia. Genet Res. 2002 October; 80(2):79-87.
54. Yamada R, Floate K D, Riegler M, O'Neill SL. Male Development Time Influences the Strength of Wolbachia-Induced Cytoplasmic Incompatibility Expression in Drosophila melanogaster. Genetics. 2007 October; 177(2):801-8.
55. Gutzwiller F, Carmo C R, Miller D E, Rice D W, Newton I L G, Hawley R S, et al. Dynamics of Wolbachia pipientis Gene Expression Across the Drosophila melanogaster Life Cycle. G3 (Bethesda). 2015 Oct. 23; 5(12):2843-56.
56. Bonneau M, Atyame C, Beji M, Justy F, Cohen-Gonsaud M, Sicard M, et al. Culex pipiens crossing type diversity is governed by an amplified and polymorphic operon of Wolbachia. Nature Communications [Internet]. 2018 December [cited 2018 Jul 20]; 9(1). Available from: http://www.nature.com/articles/s41467-017-02749-w
57. Charlat S, Calmet C, Mercot H. On the mod resc Model and the Evolution of Wolbachia Compatibility Types. Genetics. 2001 Dec. 1; 159(4):1415-22.
58. Bourtzis K, Dobson S L, Braig H R, O'Neill S L. Rescuing Wolbachia have been overlooked. Nature. 1998 February; 391(6670):852-3.
59. Zabalou S, Apostolaki A, Pattas S, Veneti Z, Paraskevopoulos C, Livadaras I, et al. Multiple rescue factors within a Wolbachia strain. Genetics. 2008 April; 178(4):2145-60.
60. Chafee M E, Funk D J, Harrison R G, Bordenstein S R. Lateral phage transfer in obligate intracellular bacteria (Wolbachia): verification from natural populations. Mol Biol Evol. 2010 March; 27(3):501-5.
61. Kent B N, Funkhouser L J, Setia S, Bordenstein S R. Evolutionary genomics of a temperate bacteriophage in an obligate intracellular bacteria (Wolbachia). PLoS ONE. 2011; 6(9):e24984.
62. Wang G H, Sun B F, Xiong T L, Wang Y K, Murfin K E, Xiao J H, et al. Bacteriophage WO Can Mediate Horizontal Gene Transfer in Endosymbiotic Wolbachia Genomes. Front Microbiol. 2016; 7:1867.
63. Wang G H, Jia L-Y, Xiao J-H, Huang D-W. Discovery of a new Wolbachia supergroup in cave spider species and the lateral transfer of phage WO among distant hosts. Infect Genet Evol. 2016; 41:1-7.
64. Wang N, Jia S, Xu H, Liu Y, Huang D-W. Multiple Horizontal Transfers of Bacteriophage WO and Host Wolbachia in Fig Wasps in a Closed Community. Front Microbiol. 2016; 7:136.
65. Yang J, Zhao X, Cheng K, Du H, Ouyang Y, Chen J, et al. A killer-protector system regulates both hybrid sterility and segregation distortion in rice. Science. 2012 Sep. 14; 337(6100):1336-40.
66. Grognet P, Lalucque H, Malagnac F, Silar P. Genes that bias Mendelian segregation. PLoS Genet. 2014; 10(5):e1004387.
67. Hammond T M, Rehard D G, Xiao H, Shiu P K T. Molecular dissection of Neurospora Spore killer meiotic drive elements. Proc Natl Acad Sci USA. 2012 Jul. 24; 109(30):12093-8.
68. Hu W, Jiang Z-D, Suo F, Zheng J-X, He W-Z, Du L-L. A large gene family in fission yeast encodes spore killers that subvert Mendel's law. Elife. 2017 20; 6.
69. Nuckolls N L, Bravo Núñez M A, Eickbush M T, Young J M, Lange J J, Yu J S, et al. wtf genes are prolific dual poison-antidote meiotic drivers. Elife. 2017 20; 6.
70. Ben-David E, Burga A, Kruglyak L. A maternal-effect selfish genetic element in Caenorhabditis elegans. Science. 2017 09; 356(6342):1051-5.
71. Seidel H S, Ailion M, Li J, van Oudenaarden A, Rockman M V, Kruglyak L. A novel sperm-delivered toxin causes late-stage embryo lethality and transmission ratio distortion in C. elegans. PLoS Biol. 2011 July; 9(7):e1001115.
72. Akbari O S, Chen C-H, Marshall J M, Huang H, Antoshechkin I, Hay B A. Novel synthetic Medea selfish genetic elements drive population replacement in Drosophila; a theoretical exploration of Medea-dependent population suppression. ACS Synth Biol. 2014 Dec. 19; 3(12):915-28.
73. Akbari O S, Matzen K D, Marshall J M, Huang H, Ward C M, Hay B A. A synthetic gene drive system for local, reversible modification and suppression of insect populations. Curr Biol. 2013 Apr. 22; 23(8):671-7.
74. Chen C-H, Huang H, Ward C M, Su J T, Schaeffer L V, Guo M, et al. A synthetic maternal-effect selfish genetic element drives population replacement in Drosophila. Science. 2007 Apr. 27; 316(5824):597-600.
75. Dodson B L, Hughes G L, Paul 0, Matacchiero A C, Kramer L D, Rasgon J L. Wolbachia Enhances West Nile Virus (WNV) Infection in the Mosquito Culex tarsalis. PLOS Neglected Tropical Diseases. 2014 Jul. 10; 8(7):e2965.
76. Hughes G L, Vega-Rodriguez J, Xue P, Rasgon J L. Wolbachia strain wAlbB enhances infection by the rodent malaria parasite Plasmodium berghei in Anopheles gambiae mosquitoes. Appl Environ Microbiol. 2012 March; 78(5):1491-5.
77. Champer J, Buchman A, Akbari O S. Cheating evolution: engineering gene drives to manipulate the fate of wild populations. Nature Reviews Genetics. 2016 March; 17(3):146-59.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

TABLE 1
Sequences of CI Factors

| | Locus | Seq. | | |
|---|---|---|---|---|
| Gene | tag | type | Length | (5′-3′) or (N-C) |
| cifA | WD0631 | nucleotide | 1425 | ATGCCAATAGAAACAAAACGTCAGGCTGAAGTGCTTAAAAAGCTA |
| | | | bp | CAAGATGTGATAAAACATACAGATCGTGACATTGCGGCTGGAAGA |
| | | | | AAGTTAGCTATTAAAAGGTGGGTCGAGACCTATATAGAGTATATC |
| | | | | AAACTTTTTAAGGATGATAAGCTGGAATTCTTATATAATGTTTTTC |
| | | | | GAGATGAAGGTTGTTGGTTAGGTACAAGGTTAAATAATACTGTTTT |
| | | | | AGGTCAGAAATTGACTGAAGAGAAAATAGGAGAAATCGATAACC |
| | | | | CACTACCAAGGTATGGTATGGCATCTAGGTACTGTATAACGGGCA |
| | | | | AGATAGGTGATTTTTTCAACAAACAGTTTGTACTCTCTAGAGGTCA |
| | | | | ATTTACTTCAGAAGAGGTAGATAGTCAAGGTAATCCGATCAGTGA |
| | | | | TCAATATGTAAGAAACATTCTGCTATCATCCATGAAGAGAAATGG |
| | | | | TCCTGTGTTTGATTTCTGGATCGATAGAGAATCTGGGGAATTAAAG |
| | | | | AAGTATGATGCAGTAGAAGGTTTTGACAGTACTGTAAAACTTAAG |
| | | | | TGGAGCGAAGGGGTAGAGTATTTTTATAATCAGTTAGAGGAAAAA |
| | | | | GATAAGGAGAAGAAGCTTACAGAAGCTATTGTTGCTCTTTCTCGTC |
| | | | | CTCAATCTGTTAAGAGAGACGCTCCTATTTTAGATTTTTGTGTAAG |
| | | | | GAATATAGGCGATAAAGATACTCTTTTACAGAAATTATTGCAGAA |
| | | | | AGATAAGGGAGTATATTTCCTTCTTGCTGAATTAATAGAGTCATGT |
| | | | | TTTTTTGATACGGTTCATGATTTGGTACAGTGCTGGTGTTATAAAG |
| | | | | GCGTTTCAGCAGGAGGAGACTGTTCGGACAAGATATTCTCACAGC |
| | | | | AAGACTATGAACTTTTTCTTTATTCACTTTCAAATGTGATGTTGAA |
| | | | | AAATCCTGAGTTAAGTGTTCAAGCTAGATCCCTTATTATGGAGATT |
| | | | | TGGAAATGTGAACGCTTTGCTGAATACAGAGAGACCTCTGTTAAT |
| | | | | ACTTCTAATTATACAGTTCCTATAAAGAGTGTACTTGGGGGATTAA |
| | | | | TCATTAATTGGAAACGAGAAGATGTTTGTAAGCCCGATAGGGAAA |
| | | | | TAGAGAAAGAAGAAATATTAGATATGATTTCATTTGCCAAAGGTT |
| | | | | GCTTTCCTGAAAAGTTTGACCTTTTTAAAGAAGTCATGATAGAAAA |
| | | | | TCTTAGAATATGTGGTAGGGAAGGAAAGAGGAAAGGTGTAGATTA |
| | | | | CGGCAAGTTTGCAGAAGAGTTATTTCTTCAGTTAGAGAAAGTAAC |
| | | | | TTTACCTTCTGTAGGTGATGGTCCTTGGAATAATTTGCGGTCTCAA |
| | | | | TCTAAGGTATCTTTGCCACTTGATGGTTCTGGTGATGGCCCACAGT |
| | | | | CTGAGTTTGAAGCTCCTAGTGTGAGTGGTATTTCTGGTTCTCATAA |
| | | | | GAAAAGAAGAATCTAG (SEQ ID NO: 1) |
| cifA | WD0631 | amino | 474 aa | MPIETKRQAEVLKKLQDVIKHTDRDIAAGRKLAIKRWVETYIEYIKLF |
| | | acid | | KDDKLEFLYNVFRDEGCWLGTRLNNTVLGQKLTEEKIGEIDNPLPRY |
| | | | | GMASRYCITGKIGDFFNKQFVLSRGQFTSEEVDSQGNPISDQYVRNIL |
| | | | | LSSMKRNGPVFDFWIDRESGELKKYDAVEGFDSTVKLKWSEGVEYF |
| | | | | YNQLEEKDKEKKLTEAIVALSRPQSVKRDAPILDFCVRNIGDKDTLLQ |
| | | | | KLLQKDKGVYFLLAELIESCFFDTVHDLVQCWCYKGVSAGGDCSDKI |
| | | | | FSQQDYELFLYSLSNVMLKNPELSVQARSLIMEIWKCERFAEYRETSV |
| | | | | NTSNYTVPIKSVLGGLIINWKREDVCKPDREIEKEEILDMISFAKGCFP |
| | | | | EKFDLFKEVMIENLRICGREGKRKGVDYGKFAEELFLQLEKVTLPSVG |
| | | | | DGPWNNLRSQSKVSLPLDGSGDGPQSEFEAPSVSGISGSHKKRRI |
| | | | | (SEQ ID NO: 2) |
| cifB | WD0632 | nucleotide | 3501 | GTGGATGGAGATCTTGATGGTTTTAGACAAGAGTTTGAATCCTTTT |
| | | | bp | TAGATCAATGTCCATTTTTCTTGTATCATGTAAGTACAGGACGTTT |
| | | | | CCTTCCTGTATTCTTTTTCAGTATGTTTGCTACTGCTCATGATGCTA |
| | | | | ATATCTTAAAAGCAAATGAGAGAGTGTATTTTCGTTTTGATAATCA |
| | | | | TGGTATTGATACAGGTGGTAGAAATAGAAATACAGGGAACCTAAA |
| | | | | AGTCGCTGTTTATCATGACGGACAGCAAGTTGTCAGATGCTACAGT |
| | | | | ATTTCTGATCGTCTTAATAGTGATGGGTTAAGGTTCAGTACAAGGG |
| | | | | AAAGAAATGCTCTAGTGCGAGAGATTAGAGGGCAAAATCCAAATT |
| | | | | TAAGGGAAGAAGACCTAAATTTTGAGCAATACAAAGTATGCATGC |
| | | | | ATGGAAAGGGCAAGAGTCAGGGAGAGGCGATTGCAACAGTATTC |
| | | | | GAGGTGATTCGTGAAAAAGATTCTCAAGGTAGAGATAGATTTGCT |
| | | | | AAATATTCAGCGTCTGAGATTAGCCTTCTTAGGCATATAGAACGCA |
| | | | | ATAGGCTTAATGGGATTAATGCGCCTGCGCCACGCAGTTTGTTGAC |
| | | | | AGTTAAGGAAATAGGAAGTATACGACTCAATCAAGATCAGAGAGT |
| | | | | ACAGCTTGGTCATTTGGTCAATTTTGTGCAAGTTGCACCGGGTCAG |
| | | | | CAAGGGATTTTCAGTTTTATGGAAGTGCTAGCAAGTAACCAAAAA |
| | | | | ATAAATATAGAACGTGGAATAAATGAAGGAATTTTGCCATACATA |
| | | | | ACTCGAATCTATCGTAGTTACCTAGGCAGCCTACAAAATGACATTC |
| | | | | AAAATCGCAGTCAAAAGTTTGAGAGTCACGGATTTTTCTTAGGTTT |
| | | | | GTTGGCAAATTTTATTCATCTCTACACAATAGATATTGACCTTGAC |
| | | | | TTGTCTCCTGGAAATTCATATGTTGCTTTTCTTATATGTCATCAGGC |
| | | | | AGAGAGAGAAAACATTCCTATCGTTATTAATGTTACTAGATGGAG |
| | | | | GACATCGTCTGATATTGCATTAAACCGCGCTAGAGCTGATGCTAA |
| | | | | AAGATTACATGTTTCTTCATTTATATCTATTCACACTGAATCAAGA |
| | | | | AATGCTGTTTGTATTGGATTAAATTTTAATCTGAATATAGATCCTTT |
| | | | | TAGTATTGATACAGTAGAGTTTTTAGAGAATAGATTTCCTTTGGTA |
| | | | | CAAAGATTATTTGAGTGTTTGGAGGATGAAGGAATTAGAGAAAAT |
| | | | | ATTAGAGATTTCTTGCTTCAACATCTTCCTAACGAAATACCAAGAA |
| | | | | ATGCAGAGAATTATAACAGAATATTTGATTGCATAACTGGTTTTGC |
| | | | | TTTTGGGAATAGTATTTTAGAAGAGTTCAGATTAGTAAACGCAGTT |
| | | | | CAACAACGTGTAAGAAAGTATATATTTAGATATGGTGATGAGAAT |
| | | | | CATGCTTTAACCATGGTCTTCCATACTCAAGGTTCTGATATAGTTA |
| | | | | TACTTCATATTAGAGATAACAACGCTGTACAACAAGGAGCCATCA |
| | | | | ATTTACAAGATCTTAATGTTGACGGAAATAATGTTCATGTACGGGA |
| | | | | AGTTTCATGCACACTTAATAATCAACTTGGCCTTAATATTCATACA |
| | | | | GATAACCTTGGTTTATATCACAATTACCAAAATAATAATGCAAATA |
| | | | | ATTTTCTTGGTGGTAATCTTGTGCAAGTGCCTAATGCTGGAAATGT |
| | | | | GCATAATGCTTTAAATCAAGTTATGAATGATGGCTGGCAAGATAG |
| | | | | ATTTCAGCATCAAGAATTATTTAGAAACATTTCTGCAGTATTAATG |
| | | | | CCAGAAGATACGCATGGCAATATGATAATAGATGTAAATAGCAAA |
| | | | | GATAAGTTTCGCTCTATACTACATGGTACATTTTATGCTAGTGATA |
| | | | | ATCCTTATAAAGTGCTTGCTATGTATAAAGTTGGTCAAACATATAG |
| | | | | TTTAAAAAGGTGGCAGGAAGAAGAAGGAGAAAGGGTAATACTTA |
| | | | | CAAGAGTTACAGAACAGAGACTAGGTCTTCTATTATTAAGACAAC |
| | | | | CTACAGCAGATACTCACCCAATTGGATATGTATTAGGATTTGCTGA |
| | | | | TAATGCAGAAGAAGTAGAACAGGAGCAAGACGAGGCAAGGTACA |
| | | | | AAATAACAGAATTGATGAGCAAACAAAGGGGATATTTGCCTATTA |
| | | | | CTTCTGGAAATGAGGTGGTTTTGTCTTATGCTGTATTTAATAGAGG |
| | | | | TGCACAGAGAGCAGAAGACTTTATATCTCTTCCACAACAAGCAGT |
| | | | | GTATGTACATAGACTTGATCGTCGTGGTCATGACTCAAGACCAGA |
| | | | | AGTATTAGTGGGACCTGAAAGTGTTATTGATGAAAATCCACCAGA |
| | | | | AAATCTATTGTCAGATCAAACTCGTGAAAATTTCAGGCGCTTTTAC |
| | | | | ATGGAAAAAAGACCAGGACAGAACTCGATTTTTTTGCTTGATATA |
| | | | | GATGATAATCTGCACGTTCCATTTAGTTACTTGCAAGGTACTAGAG |
| | | | | CACAGGCAATAGAAACATTAAGGTCAAGAATAAGGGGAGGTGGT |
| | | | | ACTTCTACAGCACAAGGAATATTACAACAAATAAACACTATCCTT |
| | | | | CGTAGAAACAACGCTCGTGAAATAGAAGATGTGCATAATCTACTT |
| | | | | GCACTAGACTTTGCAACAGAAAATCAAAATTTCCGTTATTGGCTAC |
| | | | | AAACTCATGACATGTTTTTCGCTGCACGACAATATACTTTCCATGA |
| | | | | TGATCGATCTAATCCAACTAATGATCGTCATGATTTTGCAATAACT |
| | | | | TCAGTAGGAGTCGATGGAAATCAAAATGATCCAACAGGTAGGGAC |
| | | | | TTATTAAGTAGTAACATAGATAACTTTAAACAAAAAGTAGATTCG |
| | | | | GGTGAAAAAGATAGATTAACTGCTATTATTAATGTAGGTAATCGT |
| | | | | CATTGGGTTACATTAGTTATTGTCCACCAAAATGGAAATTATTATG |
| | | | | GGTATTATGCTGATTCACTTGGTCCAGATAGTCGTATTGACAATAA |
| | | | | TATTCGAGGAGCTTTAAGAGAATGTGATATTAGCGATGATAATGT |
| | | | | CCATGATGTTTCCGTTCATCAGCAAACAGATGGCCATAATTGTGGC |
| | | | | ATATGGGCATACGAAAATGCTAGGGATATTAACCAAGCTATTGAT |
| | | | | CAAGCTTTACAGGGAAATAGTAACTTTGGAGAGAAAGGTGAAGGT |
| | | | | ATTATAGGTTATATACGTGGTCTTCTTAGTGCAGGAATTGGAAATG |
| | | | | ACACTAGACAACCTCAAAGAAATGAACAATACTTTAGAAATCGGA |
| | | | | GAAGAAATATTTCACAATTATTCCAAAATGATTCTCTATCTTCTCC |
| | | | | TAGGGGTAGATTGATTCAAGGTCGTCCAGGAATTCAACATGAAAT |
| | | | | TGATCCATTACTATTACAATTTTTAGAACTCCAATATCCACAGCGT |
| | | | | GGAGGTGGGGGAGCATTGCAATTAGGCGGAGAAAGAGTGATATC |
| | | | | AATTGATTTTGGTCCGCAATCTGTATTGGATGAAATTGATGGAGTG |
| | | | | AATAGAGTTTATGATCATAGCAATGGTAGAGGCAGTAGGTAG |
| | | | | (SEQ ID NO: 3) |
| cifB | WD0632 | amino | 1166 | MDGDLDGFRQEFESFLDQCPFFLYHVSTGRFLPVFFFSMFATAHDANI |
| | | acid | aa | LKANERVYFRFDNHGIDTGGRNRNTGNLKVAVYHDGQQVVRCYSIS |
| | | | | DRLNSDGLRFSTRERNALVREIRGQNPNLREEDLNFEQYKVCMHGKG |
| | | | | KSQGEAIATVFEVIREKDSQGRDRFAKYSASEISLLRHIERNRLNGINA |
| | | | | PAPRSLLTVKEIGSIRLNQDQRVQLGHLVNFVQVAPGQQGIFSFMEVL |
| | | | | ASNQKINIERGINEGILPYITRIYRSYLGSLQNDIQNRSQKFESHGFFLG |
| | | | | LLANFIHLYTIDIDLDLSPGNSYVAFLICHQAERENIPIVINVTRWRTSS |
| | | | | DIALNRARADAKRLHVSSFISIHTESRNAVCIGLNFNLNIDPFSIDTVEF |
| | | | | LENRFPLVQRLFECLEDEGIRENIRDFLLQHLPNEIPRNAENYNRIFDCI |
| | | | | TGFAFGNSILEEFRLVNAVQQRVRKYIFRYGDENHALTMVFHTQGSDI |
| | | | | VILHIRDNNAVQQGAINLQDLNVDGNNVHVREVSCTLNNQLGLNIHT |
| | | | | DNLGLYHNYQNNNANNFLGGNLVQVPNAGNVHNALNQVMNDGWQ |
| | | | | DRFQHQELFRNISAVLMPEDTHGNMIIDVNSKDKFRSILHGTFYASDN |
| | | | | PYKVLAMYKVGQTYSLKRWQEEEGERVILTRVTEQRLGLLLLRQPTA |
| | | | | DTHPIGYVLGFADNAEEVEQEQDEARYKITELMSKQRGYLPITSGNEV |
| | | | | VLSYAVFNRGAQRAEDFISLPQQAVYVHRLDRRGHDSRPEVLVGPES |
| | | | | VIDENPPENLLSDQTRENFRRFYMEKRPGQNSIFLLDIDDNLHVPFSYL |
| | | | | QGTRAQMETLRSRIRGGGTSTAQGILQQINTILRRNNAREIEDVHNLL |
| | | | | ALDFATENQNFRYWLQTHDMFFAARQYTFHDDRSNPTNDRHDFAIT |
| | | | | SVGVDGNQNDPTGRDLLSSNIDNFKQKVDSGEKDRLTAIINVGNRHW |
| | | | | VTLVIVHQNGNYYGYYADSLGPDSRIDNNIRGALRECDISDDNVHDV |
| | | | | SVHQQTDGHNCGIWAYENARDINQAIDQALQGNSNFGEKGEGIIGYI |
| | | | | RGLLSAGIGNDTRQPQRNEQYFRNRRRNISQLFQNDSLSSPRGRLIQG |
| | | | | RPGIQHEIDPLLLQFLELQYPQRGGGGALQLGGERVISIDFGPQSVLDE |
| | | | | IDGVNRVYDHSNGRGSR (SEQ ID NO: 4) |
| cidA | WP0282 | nucleotide | 1476 | ATGCCAACACAGAAAGAGCTTCGGGATACGATGTCCAAAAAATTA |
| | | | bp | CAGGAAGCTATTAAACATCCAGATCCAGCAGTTGTTGCCGGGAGG |
| | | | | AAGTCAGCTATCAAGAGATGGGTGGGAGTCCTTCAAGATAACTTT |
| | | | | ATGGAGCACATAAAATACTTTAAGGGTGATAAGTTGAAGTTTTTG |
| | | | | CACAATGTATTTCAAGATGAAGGTTGCTGGTCAGGTGTAAGGTTG |
| | | | | GATAATGCTGCTTTAGGTCAAAGGTTTACTGAAGAAAAAATAGGT |
| | | | | GGAATAGATAATCCACTTCGCAAATATGAGATGGCTTGTAGTTACT |
| | | | | GTGTGGTGGATAAAATTCATCCTCTCTTTCAAAAAAGATTTGAATC |
| | | | | TTATAGGAACAAGTTTCCTCCTGGTGCATTTGATGGTAAAACTGAA |
| | | | | ACTGAATTTGGCAAATACGTACGAAACTCGTTACTAGATAGCATA |
| | | | | AAGAGGAAAGGTCCTGTATTTGATTTCTGGATTGATAGAGAATCT |
| | | | | GGGGAATTAAAGAAGTATGATGCAGTAGAAGGTTTTGACAGTGCT |
| | | | | GTAAAATTTAAGTGGAGTGAAGGGGTAGAGTATTTTTATAATCATT |
| | | | | TAAAAGAAGAAGATAAGGAAAAGAAGCTCACAGAAGCTATTCTT |
| | | | | GCTCTTTCTCGCGTTCAATCTGTTGAGAAAGACGCCCCTATTTTAG |
| | | | | ATTTTTGTGTAAATAAGATAGTCGATAAAGATACTCTTTTACAGAA |
| | | | | ATTATCACAGAAAGATAAAGGAGTATATTCCCTTTTTGCTGAATTA |
| | | | | ATAGAGTCATGTTTTTTTGATACGGTTCATGATTTGGTACAGTGCT |
| | | | | GGTGTTATAAAGAAGTTTCAGCAGGAGGAGACCATTCAGAGAAAA |
| | | | | TATTCTCACAGCGAGACTATGAGCTTTTTCTTTCCTCTCTTTCAGAC |
| | | | | ACAATGTTGAAAAATCCTGAGTTAAGCGTTCAAGCTAGATCTCTTA |
| | | | | TTATGGAATTTTGGGAATGTGGTAGCTTGTATCAATACAGAAAAG |
| | | | | CTGCTGTTAATACTTCTAATTATACAGTTCCTACAAGTGGTGTATTT |
| | | | | GCAGAGTTAATAGTCAATTGGAGACGAGAAGACATTTATAAGACT |
| | | | | GATGAAGAAAAAGAAATAGAGAAAAAAGAAATATTAGATATGAT |
| | | | | GTCATTTGCCAAAGATTGCTTTCCTGAAAAGTTTGAGCTCTTTAAA |
| | | | | AAACTAATAATAAGAGACCTTAGATTATGCGGTAGGGAAGGTAAA |
| | | | | AGAGTAAATGTAGATTACGGTCTGTTTGCAGAAGAATTATTCTCTG |
| | | | | AGTTAGAGAAAACAATTTTACCACCTGGTCCTGTAGGTGATGGTCC |
| | | | | TTGCAGTAATTTGCGATCACGATCTAAAGCTCATGGTAGTAAGAA |
| | | | | AACAACTTTGCCAGTTGATGATAGTCCGCAGTCTGAGCTTGGAACT |
| | | | | CCTAGTGTAAGTGGTGTTTCTTCTTATAAGAAAAAAAGCGTCTTTA |
| | | | | CGCTTAGTGGTAATAAGTAA (SEQ ID NO: 5) |
| cidA | WP0282 | amino | 491 aa | MPTQKELRDTMSKKLQEAIKHPDPAVVAGRKSAIKRWVGVLQDNFM |
| | | acid | | EHIKYFKGDKLKFLHNVFQDEGCWSGVRLDNAALGQRFTEEKIGGID |
| | | | | NPLRKYEMACSYCVVDKIHPLFQKRFESYRNKFPPGAFDGKTETEFG |
| | | | | KYVRNSLLDSIKRKGPVFDFWIDRESGELKKYDAVEGFDSAVKFKWS |
| | | | | EGVEYFYNHLKEEDKEKKLTEAILALSRVQSVEKDAPILDFCVNKIVD |
| | | | | KDTLLQKLSQKDKGVYSLFAELIESCFFDTVHDLVQCWCYKEVSAGG |
| | | | | DHSEKIFSQRDYELFLSSLSDTMLKNPELSVQARSLIMEFWECGSLYQ |
| | | | | YRKAAVNTSNYTVPTSGVFAELIVNWRREDIYKTDEEKEIEKKEILDM |
| | | | | MSFAKDCFPEKFELFKKLIIRDLRLCGREGKRVNVDYGLFAEELFSEL |
| | | | | EKTILPPGPVGDGPCSNLRSRSKAHGSKKTTLPVDDSPQSELGTPSVSG |
| | | | | VSSYKKKSVFTLSGNK (SEQ ID NO: 6) |
| cidB | WP0283 | nucleotide | 3525 | ATGAGTAATGGTGATGGACTTATTAGGAGTTTGGTGGATGGAGAT |
| | | | bp | CTTGAAGGATTCAGACAAGGATTTGAATCTTTTTTAGATCAATGTC |
| | | | | CATCTTTCTTGTATCATGTAAGTGCAGGTCGTTTCCTTCCTGTATTC |
| | | | | TTTTTTAGTATGTTTTCTACTGCACATGATGCTAATATCTTAAATGC |
| | | | | AAATGAGAGAGTCTATTTTCGTTTTGATAACCATGGTGTTAATCCA |
| | | | | CGTAATGGTGAAAATCGAAATACGGCAAACCTAAAAGTTGCTGTT |
| | | | | TATCGTGACGGACAGCAAGTTGTCAGATGCTACAGTATTTCTGATC |
| | | | | GTCCTAATAGTGATGGGTTGAGGTTCAGTACAAGGGAGAGAAATG |
| | | | | CTCTAGTACAAGAGATTAGACGGCAAAATCCAAATTTAAGGGAAG |
| | | | | AAGACCTAAATTTTGAGCAATACAAAGTATGCATGCACGGAAAGG |
| | | | | GCAAGAGTCAGGGAGAGGCAATTGCAACGGTATTCGAGGTAATTC |
| | | | | GTGAAAAAGATCGTCAAGGTAGGGATAAATTTGCCAAATATTCAG |
| | | | | CATCTGAGGTTCATTTCTTGAGGCAACTCTTTAGAAATCACAGATT |
| | | | | AACAATTAAGGAAATAGAAGGAAGACAACTCAATCAAAATCAGC |
| | | | | TCAGACAACTTGGTAGGTCAGTCAATTTTACACGAGTAGAACCAG |
| | | | | GTCAGCAGAGGATTGACAACTTTATGGAAATGCTAGCAAGTAACC |
| | | | | AAAGACAAGATGTAAGGGATTCTCTCCGAGGAGATATTTTAGAAT |
| | | | | ATGTAACTGATACCTATAACAATTATAGGGCACAGATAGAAAATA |
| | | | | ATATTGAAGGTCGCAGTCAAAAGTTTGAGAGTCATGGGTTTTTATT |
| | | | | AGGTTTCTTAGCAAATTTTAGTCATCGCTACACAATAGGCGTCGAT |
| | | | | CTTGACTTATCTCCTAGAAACTCACATGTTGCATTTCTTGTACGTCA |
| | | | | TCAAGTAGAAAGAGAAAATATTCCTATTGTTATTAATCTTGCTACA |
| | | | | AGGGCACCGCCCTATATCGCATTAAACCGCGCCAGAAGTCACGCT |
| | | | | GAAAGATTGCATGTTTTTTCATTTATACCTATCCATACTGAATCAA |
| | | | | GAAATACTGTCTGTGTTGGATTAAATTTTAATTTAAATCTAGATCC |
| | | | | TTTTAGTGTTGATACAGTAGGGCTTCAACAGGATAGATTTCCTTTA |
| | | | | GTACAAAGATTATTTGAGTGTTTGGAGAATGAAGGAATTAGAGAA |
| | | | | AATATTAGAGATTTCTTGCTTCACCATCTTCCTGCTGAAATACCAA |
| | | | | GAAATGCAGAGAATTATGATAGAATATTTGATTGCATAACTGGTTT |
| | | | | TGCTTTTGGGAATAGTGCTTTTGATAGGCACCCTTTAGAACTAGAA |
| | | | | GAGGAAGACGAAGCACCTATAACAAAGTACATATTTAGACATGGT |
| | | | | GATGAGGGTTTAAGATGTTTAACTATGGTCTTTCATGCTGAAGGTT |
| | | | | CTGATATAGTTATACTTCATATTAGAGCTCACGATGCGCAACAACA |
| | | | | AGGAGCCATCAATTTACAGACTCTTAATGTTAATGGAAATGATGTT |
| | | | | CATGTGTGGGAAGTTTCATGCACACTTAATAATCAACTTGAACTAG |
| | | | | ATATTGATCTACCAAATGACCTTGGTTTATATCACGATTACCAAAA |
| | | | | TAATAATGCAAATAATTTTCTTGCTGGTGATCTTGTACAAGTGCCC |
| | | | | AATACTGAAAATGTACATAATACTTTAAATCAAGTTGTGAATGAT |
| | | | | GGCTGGAAAAATATAGCTCAGCATAGAGGATTATTTCAAGAGATC |
| | | | | TCTGGAGCATTGATGCCGCTTGTGGATACAATAAATGTTAATAGTG |
| | | | | AGGATAAGTTCCGTTCTATACTACATGGTACATTTTATGCTAGTGA |
| | | | | TAATCCTTATAAAGTGCTTGCTATGTATAAAGTTGGTCAAACATAT |
| | | | | AGTTTAAAAAGGGGGCAGGAAGAAGAAGGAGAAAGGGTAATACT |
| | | | | CACAAGAATTACAGAACAGAGATTAGATCTTTTATTATTAAGACA |
| | | | | ACCTAGAGAGAATGACCTAGATACTCACCCAATTGGATATGTGTT |
| | | | | AAGACTTGCTAATAATGCAGAAGAAGTAGGACAACAGCAAAATG |
| | | | | ATGCGAGACAAGAAATCGGAAGACTTAAGAAACAACACAGAGGA |
| | | | | TTTATACCTATTACTTCTGGAAATGAGGTGGTTTTGTTTCCTATTGT |
| | | | | GTTTAATAGAGATGCACACGAAGCAGGTAATCTTATACTTTTCCCA |
| | | | | GAAGGGATAGGAAGAGAAGAGCATGTACACAGGCTTGATCGTCAT |
| | | | | GTTCGCAGCTCAAGACCAGGAGGATTAGTGGGACCTGAAAGTGTT |
| | | | | ATTGATGAAAATCCACCAGAAGGTCTATTATCAGATCAGACTCGT |
| | | | | GAAAACTTTAGGCGTTTTTACGAAGAAAAAGCACCAGGACAAAAT |
| | | | | TCGATTTTTTTGCTTGATATAGGCGACAATCTACATGTTCCCTTTAG |
| | | | | TTACTTGCAAGGTACTAGAGCACAGGTAATAGAAACATTAAAGTC |
| | | | | AAGAATAAGGGGAGGTGGTACTCCTACAGCACAAGGAATATTACA |
| | | | | ACAAATAAATGCTATCCTTCGTAGAAACAACGCTCGTGAGATAGA |
| | | | | AGATGTGCATGATCTACTTGCACTAGACTTTGCAACAGATAATCAA |
| | | | | AATTATCGTTATTGGCTACAAACTCATGACATGTTTTTCGCTGCAC |
| | | | | GACAATATACTTTCCTTGATAATCAATCTCATTCAACTAATGATCA |
| | | | | TTATGGTTTTGAAATAACTTCAGTAGGAGTCGATGGAAATCAAAA |
| | | | | TGATCCAACAGGTAGGGGCTTATTAAGTAGTCACATAACTAACTTT |
| | | | | AAACAAAAAGTAGATTCGGGTGAAAAAGATAGATTAATTGCTATT |
| | | | | ATTAATGTAGGTAATCGTCATTGGGTTACATTAGTTATTGTACACC |
| | | | | AAAATGGAAATTATTATGGGTATTATGCTGATTCACTTGGTCCAGA |
| | | | | TAGTGGTATTGACAATAATATTCGAGGAGCTTTAAGAGAATGTGA |
| | | | | TATTAACGATGATAATGTCCATAATATTTCCGTTCATCAGCAAACA |
| | | | | GATGGCCATAATTGTGGCATATGGGTATACGAAAATGCTAGGGAT |
| | | | | ATTAACCAAGCTATTGATCAAGCTTTACAGGGAAATAATAACTTTG |
| | | | | GAGAGAAAGGTGAAGGTATTATAGGTTATATACGTGGTCTTCTTA |
| | | | | GTGCAGGCATTGGAAATGACACTAGACAACCTCGAAGAAATGAAC |
| | | | | AATACTTTGAAGATCGGAGAAGAGATATTTCACAATTACTCCAAA |
| | | | | ATGATCCTAACTTACCTTCTCGCCGGAGTGATTTAATTCAAGCTCA |
| | | | | TCCAGGAATTCAACATGAAATTGATCCATTACTATTACAATTTTTA |
| | | | | GGACTCCAATACCCACAGCGTGGAGGTGGAGGAGCATTACAATTA |
| | | | | GGCGGAGAAAGAGTGATATCAATTGATTTTGGTAACCCGCAGTCT |
| | | | | GCATTAGATAAAATTGATGGAGTGAGTAGAGTTTATAACCATAGC |
| | | | | AATAGTAGAGGTAGTAGGTAG (SEQ ID NO: 7) |
| cidB | WP02 | amino | 1174 | MSNGDGLIRSLVDGDLEGFRQGFESFLDQCPSFLYHVSAGRFLPVFFF |
| | 83 | acid | aa | SMFSTAHDANILNANERVYFRFDNHGVNPRNGENRNTANLKVAVYR |
| | | | | DGQQVVRCYSISDRPNSDGLRFSTRERNALVQEIRRQNPNLREEDLNF |
| | | | | EQYKVCMHGKGKSQGEAIATVFEVIREKDRQGRDKFAKYSASEVHF |
| | | | | LRQLFRNHRLTIKEIEGRQLNQNQLRQLGRSVNFTRVEPGQQRIDNFM |
| | | | | EMLASNQRQDVRDSLRGDILEYVTDTYNNYRAQIENNIEGRSQKFES |
| | | | | HGFLLGFLANFSHRYTIGVDLDLSPRNSHVAFLVRHQVERENIPIVINL |
| | | | | ATRAPPYIALNRARSHAERLHVFSFIPIHTESRNTVCVGLNFNLNLDPF |
| | | | | SVDTVGLQQDRFPLVQRLFECLENEGIRENIRDFLLHHLPAEIPRNAEN |
| | | | | YDRIFDCITGFAFGNSAFDRHPLELEEEDEAPITKYIFRHGDEGLRCLT |
| | | | | MVFHAEGSDIVILHIRAHDAQQQGAINLQTLNVNGNDVHVWEVSCTL |
| | | | | NNQLELDIDLPNDLGLYHDYQNNNANNFLAGDLVQVPNTENVHNTL |
| | | | | NQVVNDGWKNIAQHRGLFQEISGALMPLVDTINVNSEDKFRSILHGT |
| | | | | FYASDNPYKVLAMYKVGQTYSLKRGQEEEGERVILTRITEQRLDLLL |
| | | | | LRQPRENDLDTHPIGYVLRLANNAEEVGQQQNDARQEIGRLKKQHR |
| | | | | GFIPITSGNEVVLFPIVFNRDAHEAGNLILFPEGIGREEHVHRLDRHVRS |
| | | | | SRPGGLVGPESVIDENPPEGLLSDQTRENFRRFYEEKAPGQNSIFLLDI |
| | | | | GDNLHVPFSYLQGTRAQVIETLKSRIRGGGTPTAQGILQQINAILRRNN |
| | | | | ARELEDVHDLLALDFATDNQNYRYWLQTHDMFFAARQYTFLDNQSH |
| | | | | STNDHYGFEITSVGVDGNQNDPTGRGLLSSHITNFKQKVDSGEKDRLI |
| | | | | AIINVGNRHWVTLVIVHQNGNYYGYYADSLGPDSGIDNNIRGALREC |
| | | | | DINDDNVHNISVHQQTDGHNCGIWVYENARDINQAIDQALQGNNNF |
| | | | | GEKGEGIIGYIRGLLSAGIGNDTRQPRRNEQYFEDRRRDISQLLQNDPN |
| | | | | LPSRRSDLIQAHPGIQHEIDPLLLQFLGLQYPQRGGGGALQLGGERVIS |
| | | | | IDFGNPQSALDKIDGVSRVYNHSNSRGSR (SEQ ID NO: 8) |
| cixA | wRi_06720 | nucleotide | 1371 | ATGCCAAAAAAGATGGAGCGTCATGCTGCAGTGCTTAGTAAGTTA |
| | | | bp | AAGAGTGTTATTCAACATACAGATTCCAAGGTCATGGCTGAAAGG |
| | | | | CGTTCAGCTATTGAAAGATGGGTAAAAACTTACATTAGGCAGGTA |
| | | | | GAATATCTTAAAGATGATAAGCTACAATTCTTATACAACATATTTC |
| | | | | GCGATGAAAGTTGTTGGTCAGGTACGAGATTGAACAATACAATCT |
| | | | | TAGGACAGAGGTTTACTGAAGAAAAAATAGGCGAAATAAAGAAC |
| | | | | CCTCTTCCTATATATGATATGGCATGTCGATACTGCGTGATAGATA |
| | | | | AAATTCCTTTGCTCTTTCAGAAGCAGTTTGAATCTTACAAAAGTAG |
| | | | | CTTCTCTTCTGAAGAGATAGATGATGATGGTAAGCCTGCAACTAGC |
| | | | | AATAACAAATATGTAAAGAGTGAGTTGTTGGGTTATATGAAGAGT |
| | | | | CAAGACCCTGTATTTAGCTTTTGGGTTGATAAAAAATCTGGAGAAT |
| | | | | TTAAGAAGCATGTCAGCGCAACAGAAGGATTTAAGAAAGCTATAG |
| | | | | AACTTAAGTGGAGCGAAGGAGTAGAATATTTTTATAGCCTTCTAA |
| | | | | ATGAAAAAGAAAGAGAAAGAGAAAGGAAAATTACTGATGCAGTT |
| | | | | ACTATATTATCCTCTGTTCAATGTGACCATAATGGTGCTGTTACTTT |
| | | | | AGACTTTTGTCTTAGTAAAATGAGCGATCAAGCAAAAAACAAGCT |
| | | | | GTTTAAAGATTCTGAGCTATCAAAAAAAGATAAAGGAGTGTACTC |
| | | | | TCTCTTTAGCGCGTTGATACATCAAGGTTTTTTTGATACGATGCAA |
| | | | | GCTATACTTCCGATGTTTAAAGATAAAATACTGGAGGATAAGATA |
| | | | | CTTTCACCTAGGAGTTATACTCTTCTTCTCTCCTCACTTTCGGACAT |
| | | | | GATGCTCGAAAATTCTGAGTCAACTATTCAAGCTAGGGAAGCTAT |
| | | | | AATGAACCTTATAAAGTGTGGTAATTTCAATAATCATGAGGGGCG |
| | | | | TGAGGAAAAAGCTGCGGTATTTTTTTCTAATGGAAGGGTTCCGATT |
| | | | | AAGCGTGCGCTTGCAGGATTGATTGTCGATTGGCAACTTGGTTGTA |
| | | | | CAAAAAAGGAAGAGGTGTTAAAGGTACTACAGTTTGCCAAAGAGT |
| | | | | TTTGTGCAGTTGAAAGTTTTATGTATTTTAAAAAATCTGTTGTTGAT |
| | | | | AACCTAAAAATGGTTGGTAGGGATGGTATGAGAAAAAATATAGAC |
| | | | | TATGGTAAATTAGCAGAAAAGTTGTTTGCTGAATTAGATACGGTAT |
| | | | | CCGTGCCTAACGGAAGAGGTGATTTTGGTGGAGCTGGTGACCCAC |
| | | | | AGTCTACACTAGGAAGCACTGAAGTTAGTAGTTTTTCTGGTCGCAA |
| | | | | TAAGTAG (SEQ ID NO: 9) |
| cixA | wRi_06720 | amino | 456 aa | MPKKMERHAAVLSKLKSVIQHTDSKVMAERRSAIERWVKTYIRQVE |
| | | acid | | YLKDDKLQFLYNIFRDESCWSGTRLNNTILGQRFTEEKIGEIKNPLPIY |
| | | | | DMACRYCVIDKIPLLFQKQFESYKSSFSSEEIDDDGKPATSNNKYVKS |
| | | | | ELLGYMKSQDPVFSFWVDKKSGEFKKHVSATEGFKKAIELKWSEGV |
| | | | | EYFYSLLNEKERERERKITDAVTILSSVQCDHNGAVTLDFCLSKMSDQ |
| | | | | AKNKLFKDSELSKKDKGVYSLFSALIHQGFFDTMQAILPMFKDKILED |
| | | | | KILSPRSYTLLLSSLSDMMLENSESTIQAREAIMNLIKCGNFNNHEGRE |
| | | | | EKAAVFFSNGRVPIKRALAGLIVDWQLGCTKKEEVLKVLQFAKEFCA |
| | | | | VESFMYFKKSVVDNLKMVGRDGMRKNIDYGKLAEKLFAELDTVSVP |
| | | | | NGRGDFGGAGDPQSTLGSTEVSSFSGRNK (SEQ ID NO: 10) |
| cixB | wRi_06710 | nucleotide | 2265 | ATGCATGGGTTAGTTAGAAGTTTAATAAATGGAAATTGTGGAGAA |
| | | | bp | TTCACGGAAAAGTTTGAATATTTCTTGGATTCATGTCCATCTTTTCT |
| | | | | GCATTCAGTTGGCAAAGATCACTTTTTTCCTGCGTTCTTTTTTGGCA |
| | | | | TGTTTGCTACTGCACATGATTCTGGTGTTGCAAACAATGATGAAAG |
| | | | | AATCTTCTTTCGTTTTGATAATGATCCAGGTAGTCCTGGAAGGGGA |
| | | | | AATCTAAAGGTTGCAATTCTAACAACTGATGGAAATAACAGAAGA |
| | | | | GTTGTAAGGTGCTATACTATTGCTGACAGAGAGAATAGCTACGGT |
| | | | | TCTAGGTTTAGCCAGCAGGAAAGGGAGCAGCTGGAAGGTATCCTG |
| | | | | CGAGATGAAGAGCTTGAATGGCAAGAGTATAAAACATTTATATGG |
| | | | | GCGGATAATCAAGGTGAAGATGAAGAAGAGGAAGCAGTAAGATG |
| | | | | TAGGATATTTCAGGCAGGACAAGGGCCGTTTACTGGAAATCATGC |
| | | | | ATCTTATTTAACTCGTAGACATAGTTTTCAAGAGATTACCAGAACA |
| | | | | CCTGGGCTGCAAAATAATTATTTACCGGATTTGATGAATCAGCTAG |
| | | | | AAAGTGATGATGCAGATGATGTACACGACACTACTGAGGAAGTGT |
| | | | | TTCAGCATATTATTGGTGTCTACGATAGATATAGTCAGGCATTGGA |
| | | | | CTTCTATGGTAGAGAGTCTGACTATCATGGTTTTGTTTCCGGTGTTT |
| | | | | TGATGCATTTTAGATATCGCAATGTAGCCAATATTTACCTTGAGCT |
| | | | | GTTTGTAGGTGGTGGATATGCAGATATTACTTCTATTGTACGTGGT |
| | | | | ACACAGAGGTTAATTAATTCTGTTCCCTGTGTAACTGAACTTAAGG |
| | | | | CAGGCAGAAGAGCAGATAGGAATGCTGGCCGTGCATTAGAGCAG |
| | | | | GCTGGAAATTATGTTAATGGATGTCCCGTTTCATCCATATCTATTC |
| | | | | CAACATTATCACCAAGAGCTGTCTCCGCTGGAGTGAATTTCGATTT |
| | | | | TGGTAACCCAGGACGTTTACAGCTTGGTGTGAGGGCTTTTTTAGCA |
| | | | | AAAGGTTCTTCTTTAATGGAAAGATTATTTGAACCTGTAGAGGATG |
| | | | | AGGAGATTGGAGAAAATGTTAGGGATTATCTACTCCATCCAGCCT |
| | | | | TTGGTGTACCTGCTGTACCAGGTATTAGGAATAGGGGTGGTGTTAA |
| | | | | CGCTAGAGATAGAAGAATATTTCTCTATACAAGTGGATTTGCTTTC |
| | | | | GCAAGTATTGCATTTGCAAAAGGAACTGTGCCAATAGAAGGAAAT |
| | | | | CGTGCAATAGTAGATAAGCACTTGTTTCACTATGACGGTAATGCA |
| | | | | AAAATGTTAGATGAGCAAAGATACAATACACAAGTAAATATTGGA |
| | | | | GATCGTGCTTTGACTATGGTTTTGCATGTATCACGAGGTAGAGACC |
| | | | | AGAAGGAGGAGGTGATCGTATTTCATGTTCGCCACGTATTGGCTA |
| | | | | ATCAACTTTTTCCGGACAATGGATTGGATCTATCGCGTTGGCCGAA |
| | | | | TGCTATGGTACATGAAGTGGTGTGTAATTTGACCATAAATAGAAG |
| | | | | GACAAGAGGAGTAAATGATAATCTTGGTTTAACTGTTAATGTAGA |
| | | | | AACATTTGACTCGCCTGCTGACTACCTGCTTGATAGAGGTAATCAG |
| | | | | CCTTTTCAAGGTGAGCTTTTACGAATAGGTGGCGTTAGTAATGTGC |
| | | | | ATCGCGCTGCAAATGTAATGATGAATACTGGCTGGGAAAATGAAG |
| | | | | ATCCAGACAGTCATGAACGGTTTTACCAAGCAATTTCCAACGTGCT |
| | | | | AAATCCACCCCAGCCAAATAATGCAGGACTCCAATCATTAGCATG |
| | | | | GGTAGTGAACAGAGATAATGCTAGAGAAGCTGGGTTTCATGCTGC |
| | | | | ATTGCATGGATTATTTTACACTTGCGATAATCCTGCTAGGGTAGTT |
| | | | | AGTGAATTTCAGGTTGGAGGAGGAGGAAAGTTAGACTTAGTATTG |
| | | | | TCACGAGCTATAGGAAGGATGGGAGGTACTTATCCGATTGGAACA |
| | | | | GAGCTAAAGTTTGCTGCCACTGAAGCAGACGTACAAAATAGAGAA |
| | | | | GAAGAAGCAGATGAACAGGTGGAGGGTTATCTGCAGAGTAGAGG |
| | | | | GTTTGATCGCATTACTGATGGAGATAAAATGGTTTTCTCGTATGCC |
| | | | | GTATTTAATGATCAAGCGCCAGCACCAGCACAAAATGTCCCAAAT |
| | | | | ACCCTTATAGCAGTTAGTAATGTTCTACGCATAAAAGATAACTTAG |
| | | | | GAATTGACACTGTGGACGACTTTCCTTATAGATAA (SEQ ID NO: 11) |
| cixB | wRi_06710 | amino | 754 aa | MHGLVRSLINGNCGEFTEKFEYFLDSCPSFLHSVGKDHFFPAFFFGMF |
| | | acid | | ATAHDSGVANNDERIFFRFDNDPGSPGRGNLKVAILTTDGNNRRVVR |
| | | | | CYTIADRENSYGSRFSQQEREQLEGILRDEELEWQEYKTFIWADNQGE |
| | | | | DEEEEAVRCRIFQAGQGPFTGNHASYLTRRHSFQEITRTPGLQNNYLP |
| | | | | DLMNQLESDDADDVHDTTEEVFQHIIGVYDRYSQALDFYGRESDYH |
| | | | | GFVSGVLMHFRYRNVANIYLELFVGGGYADITSIVRGTQRLINSVPCV |
| | | | | TELKAGRRADRNAGRALEQAGNYVNGCPVSSISIPTLSPRAVSAGVNF |
| | | | | DFGNPGRLQLGVRAFLAKGSSLMERLFEPVEDEEIGENVRDYLLHPAF |
| | | | | GVPAVPGIRNRGGVNARDRRIFLYTSGFAFASIAFAKGTVPIEGNRAIV |
| | | | | DKHLFHYDGNAKMLDEQRYNTQVNIGDRALTMVLHVSRGRDQKEE |
| | | | | VIVFHVRHVLANQLFPDNGLDLSRWPNAMVHEVVCNLTINRRTRGV |
| | | | | NDNLGLTVNVETFDSPADYLLDRGNQPFQGELLRIGGVSNVHRAANV |
| | | | | MMNTGWENEDPDSHERFYQAISNVLNPPQPNNAGLQSLAWVVNRD |
| | | | | NAREAGFHAALHGLFYTCDNPARVVSEFQVGGGGKLDLVLSRAIGR |
| | | | | MGGTYPIGTELKFAATEADVQNREEEADEQVEGYLQSRGFDRITDGD |
| | | | | KMVFSYAVFNDQAPAPAQNVPNTLIAVSNVLRIKDNLGIDTVDDFPY |
| | | | | R (SEQ ID NO: 12) |
| cinA | wNo_01990 | nucleotide | 1473 | ATGCCAAAAAGTAAAACTAAACGTGGAACGGAAGATTTGAAGGG |
| | | | bp | TAATGCAGGCCCAAGCAAAAGATCTCGTCTCAGTTCTGATCCTAA |
| | | | | AAAAAATAAAGAGATTATCTCTAGCAAAGTAATAAGTAAGCTGAA |
| | | | | GGATGTTGTTAAAGGTGATAGAACTTCAGCTATTGAGGAATGGGT |
| | | | | CAAGGCTCACCCTGTCACAGTAGAGGGTCTAATCGTTGAGCAATC |
| | | | | GGACCTCTTATGTAATGCGTTTCGTGATGAATCTTGTTGGTCAGGT |
| | | | | GCGACACTAGATGTTGCTAAATTGGTAGGAGAATTAGCTAAATCA |
| | | | | GGTGTGTTGAATCCATTTGCTATATATAAAATAGCATGTATTGAGT |
| | | | | GTGTAGAGAGTGAAATTAAGCAATTATTTGACAAGGCGTTAGAGT |
| | | | | CTTTTAGATCTGACTTATCTCATAAAGGTGCATGTGAGGAAGATAG |
| | | | | GAATTTAGCTTGCAGTGATAAGCTTGCAAGAGTTGAATTGTTAAGT |
| | | | | TCCATGGGAAGACGTGATCCTGTTTTCAATTTCTGGATTGATCAAG |
| | | | | AATCAGGTAACCTTAGAGAAAATATAGAAGCAGAAGATGGATTTA |
| | | | | ATAAGGCTGTAGATTTCAAGTGGAGTAAGGGAGTGGAACACTTCT |
| | | | | ATAATCGTCTGTGTTCTGAAGAAAAATTAGTGAAAGAAGAGAGAG |
| | | | | AAAAATTGCTAGTTTCTGCTATTGCAAAATTATCTCCATTGCAATC |
| | | | | TAGCTATAAACTTGCTTCTACCTTAAATTCCCTTCTAGGTAAAGTC |
| | | | | ATAAGCGCAAAAGTAGATCATAAGTCACTACTTGGGCTACCGAAT |
| | | | | AAGAGAGATAGGGGTGTGATCTATCGTCCTCTTAGTTACTTAGTAG |
| | | | | AGCACGGTTTTCTTTGCACAACTAAGTATGTTATCCAGTACTTGAG |
| | | | | CGAGGGATGTTCAAGATCTGAAGTAGAGAAAATGCTTTCACCTAG |
| | | | | AGGATATGCACATCTTCTCTCATCGCTTTCATTTGTTGTAGTTTCTA |
| | | | | AAGATTATGACTTGGATAACAGGAATGAAGCAAGGTCAGCTATTA |
| | | | | GCAGTCTTTGGGAATCTAGTGTATTTAACCAAAATAAAATAAATGT |
| | | | | TGTCGATCCTTTTAAAGATAGGATTGCTTTTGTTGCAATGGAAAAT |
| | | | | GCAATTTCAAATTTGATTGTAGATCAGGAGAACAGTAAGGATACT |
| | | | | CAAAGTGCTGGCGATGGTGAAAAAGTTGATTTGGTCTTGAGTATTT |
| | | | | TAAAGTTTGCTAAAGATTGTTGTTCAGACAAAAGCTTTAAATCATT |
| | | | | AAAAGCGAGGATAGCAAATAGTTTAGATAAAACAAGGAATTCTAA |
| | | | | GATGATAGATGCAACTAGCTCCTGCAATTTAATAGAAGAGTTGTG |
| | | | | TAAGTCAGCGAGAAATTTGAATTTATTCTCTGCTAGCACTGAAGGT |
| | | | | CCTCAATCTACGTTAGTGGGTACTAATGTTAGTATTTCGCCTGCTG |
| | | | | CAGTTGTTAACAAATAG (SEQ ID NO: 13) |
| cinA | wNo_01990 | amino | 490 aa | MPKSKTKRGTEDLKGNAGPSKRSRLSSDPKKNKEIISSKVISKLKDVV |
| | | acid | | KGDRTSAIEEWVKAHPVTVEGLIVEQSDLLCNAFRDESCWSGATLDV |
| | | | | AKLVGELAKSGVLNPFAIYKIACIECVESEIKQLFDKALESFRSDLSHK |
| | | | | GACEEDRNLACSDKLARVELLSSMGRRDPVFNFWIDQESGNLRENIE |
| | | | | AEDGFNKAVDFKWSKGVEHFYNRLCSEEKLVKEEREKLLVSAIAKLS |
| | | | | PLQSSYKLASTLNSLLGKVISAKVDHKSLLGLPNKRDRGVIYRPLSYL |
| | | | | VEHGFLCTTKYVIQYLSEGCSRSEVEKMLSPRGYAHLLSSLSFVVVSK |
| | | | | DYDLDNRNEARSAISSLWESSVFNQNKINVVDPFKDRIAFVAMENAIS |
| | | | | NLIVDQENSKDTQSAGDGEKVDLVLSILKFAKDCCSDKSFKSLKARIA |
| | | | | NSLDKTRNSKMIDATSSCNLIEELCKSARNLNLFSASTEGPQSTLVGT |
| | | | | NVSISPAAVVNK (SEQ ID NO: 14) |
| cinB | wNo_01980 | nucleotide | 2091 | ATGCATGGTAATAATGAAGATCGTGAATTAGTTAGGGCTTTATTAA |
| | | | bp | GTGGAGGTTGTGATGAGTTTAGTAGACAATTTGTAGGTTTTTTAAA |
| | | | | CAACTGTCCATCTTTTTTGCATTCGGCTAATAAGCCTGGCTTTTTTC |
| | | | | CTACATTCTTTTTTGGTATGTTTTCTACTGCACATGATGCAGGTATA |
| | | | | TTAGTTGAAGGTGAAAGAGTCTATTTTCGTTTTGACAATTATGGAA |
| | | | | ATCTAAAAGTTGCTGTTCTCACTAATAAAGAAAATAGAAGAATAG |
| | | | | TCAGGTGTTATACTGTTGCTGATAATGAGAACAGCCCTGGGTCAA |
| | | | | GGTTTAGTGCAGAAGAGAAGCAGCAGGTAGAAGAGAATCTTCCAC |
| | | | | AAGAATTACAGGAAGATGAGGATCTGGATTGGGAAGAGTATAAA |
| | | | | ATATTTCGGTTTGGAGAAGAATGTAGGTTTATTCATGAAATAGATA |
| | | | | GATTTCCTCAACGTGATGAACCTGGAGCTCCAATTTTTCATGAAAT |
| | | | | TAACCCAATCAGAGAACAAGGTGAATTGTTAGACCTGATGAGTGA |
| | | | | GTTGGCAAATGACGATACAGGAGAAGTGCGTACTAATGTTAAAAG |
| | | | | AATTTTGGAATATGTTATTGATATCCATGATGAACATGAAGATAGC |
| | | | | TTAGTGTTTCGTGCAGAGTCTGACTACCACGGTTTTCTGTGTGGGT |
| | | | | TTTTAGTAAATTTTAGATACCGAGCTTTGGCTGATTTCTACCCAGA |
| | | | | GCTACTTATAGGAAAAGGTTATGCAGATGTTGTTTTGCTTGTTCGT |
| | | | | GGTGTTGATCAGACAAATGATTCGGTTCCAATTATAATTGAGTTGA |
| | | | | AGGTTGGTGATGAGGAAGGATTAGAGCAAGCTAAAGATTATGCTA |
| | | | | AAAGTTGTTCTGTTTCGTCTTTGCCTATTCATACCTCATCACCAAGT |
| | | | | GCTGTTTGTGTAGCGTTAAATTTTCAATTACGTGGAGGTGCTGGTC |
| | | | | TCCGAACTTCTGTGCAGGCCTTTTCAGAAGGTGGTCTTTCCTTAAT |
| | | | | ACCGGGTTTACTACATCCTCATGGAAATGGAGTTAGGGGAAATGT |
| | | | | AAAACGTTTTTTACAACCCATAGCATCAGAGTTCACTCAATCGCCT |
| | | | | CATTGTAACACTTTTTCCTGTACTTCATCGTTTGTTTTTGGAAATGT |
| | | | | TTTATCTACAAGGAGGGACTTAGAAACAAATGATGGGCGGGAGGT |
| | | | | AAGGGTTACCAAGTATCTATTTAACCACTCTCAGGGAGAGAAAAT |
| | | | | GAAACGTACAGGTGGTAGAGGAGATGCAGCAGATATTGTAAGCCA |
| | | | | TGCGTTAACTTTAGCTCTATTTTTATCAAATATTGGTTTTGTTGTGC |
| | | | | TTCACATTTTTCGTCGTTTAAAGTGGCAGACTTTACCAGACAAGGC |
| | | | | ATTGAACCTGTCGTTACTGCCTCAAGCCACAGATGATGCTAAGGTG |
| | | | | CGTCAAGTACTTTGTGAAGTAGATGTCCAGGGTCATCTGGAAGTG |
| | | | | GCTTCTGCAAAGAAATTCGAATCACTACGTGCTTACTCACGTTCTC |
| | | | | ATAGTGAAGGTTATTTCGAGGGAAGGTTTTCAGAACAAATGGGTA |
| | | | | ATGTTAGGAATTTACATCAACTTGCAGATCAGTTGATGAGTGCTGA |
| | | | | GCCTAATTTTGGTAATGATGGTAATGTTAATGGTGAGTACAGGGCT |
| | | | | AGGTATGAAGTTTTATTTAATGAGATTTCTCGTCTGTTGTCTCCGTT |
| | | | | ATTAAATGGAAACCGTCTACTCGTGAACAATGAAGCTAAATTTCA |
| | | | | GGCTTTGTTGCGTGGAATATTTCAAAATTGCGATAATCCTGCCAAG |
| | | | | GTAATTATTGAGTTCCAGCTACAGAGAGGAAGGAAAATAGACCTA |
| | | | | GTATTATCAAAATCTGCGGAAAATGATGATACTCATCCAATTGGA |
| | | | | ATAGAGTTGAAGTATGCTAACACCGCAGAACAAGTTGAACGAAAA |
| | | | | AGGGTGGAGGCAAATCGACAGTTAAGTGAATACGAATTTTGTGGA |
| | | | | GGATGCAAGCGTATTACTGGGGGAGATGCGATGGTTTTGTTATAC |
| | | | | GCTATATTAAATGCTGTAGGACAAGAGCAGGATCTGATATTGATT |
| | | | | GGTGGGCTTCGTAGAGCATCTGGGTTTTCTAGATGA (SEQ ID |
| | | | | NO: 15) |
| cinB | wNo_01980 | amino | 696 aa | MHGNNEDRELVRALLSGGCDEFSRQFVGFLNNCPSFLHSANKPGFFP |
| | | acid | | TFFFGMFSTAHDAGILVEGERVYFRFDNYGNLKVAVLTNKENRRIVR |
| | | | | CYTVADNENSPGSRFSAEEKQQVEENLPQELQEDEDLDWEEYKIFRF |
| | | | | GEECRFIHEIDRFPQRDEPGAPIFHEINPIREQGELLDLMSELANDDTGE |
| | | | | VRTNVKRILEYVIDIHDEHEDSLVFRAESDYHGFLCGFLVNFRYRALA |
| | | | | DFYPELLIGKGYADVVLLVRGVDQTNDSVPIIIELKVGDEEGLEQAKD |
| | | | | YAKSCSVSSLPIHTSSPSAVCVALNFQLRGGAGLRTSVQAFSEGGLSLI |
| | | | | PGLLHPHGNGVRGNVKRFLQPIASEFTQSPHCNTFSCTSSFVFGNVLST |
| | | | | RRDLETNDGREVRVTKYLFNHSQGEKMKRTGGRGDAADIVSHALTL |
| | | | | ALFLSNIGFVVLHIFRRLKWQTLPDKALNLSLLPQATDDAKVRQVLCE |
| | | | | VDVQGHLEVASAKKFESLRAYSRSHSEGYFEGRFSEQMGNVRNLHQ |
| | | | | LADQLMSAEPNFGNDGNVNGEYRARYEVLFNEISRLLSPLLNGNRLL |
| | | | | VNNEAKFQALLRGIFQNCDNPAKVIIEFQLQRGRKIDLVLSKSAENDD |
| | | | | THPIGIELKYANTAEQVERKRVEANRQLSEYEFCGGCKRITGGDAMV |
| | | | | LLYAILNAVGQEQDLILIGGLRRASGFSR (SEQ ID NO: 16) |
| CinA | wPa_0294 | nucleotide | | ATGGAATCTGGTTTGGATCACAATTACAATAAAATACTTGATATAT |
| wPip | | | | TAAAAGGTGCTATTAAAGGCGACGATAATCAAGTTAAAGCAAGAA |
| | | | | AACACCTTAGAGTAGAAAGATGGTTGAGGGCTTATATTCAATTAA |
| | | | | TTGAAGATTTTGATGAGGAAAAACTAATTTTTTTTTCTGATATATT |
| | | | | CTCTGATAATTCTTGTTGGGATGGAATAAAATTAAAGAATAAAGC |
| | | | | TGTTGGTGAAAGGCTAACTGAAGAAAAAAATAAAAATGGAAAAG |
| | | | | AAAATCCGCTTGATCTTGCAGATAGATATTACTTGGCATGTAAATA |
| | | | | TTGTCTAGAAGATAAGATTCCTGGATTATTTGAACAAGTATTTATG |
| | | | | AGATTTAAGAGAAGTGCCTTTGAAGAAGATGGATCTGATGATGAT |
| | | | | CTGAGAAGAGAATTATTGGAAAATATCGAAGAAACTAGCCCTATA |
| | | | | GAAGCTTTCTGGTCTTTTCTTATTGATAAGCAGATTGGAAAACTAA |
| | | | | ACGAATATAAATCAGTTGAAGGTTTGCAAAAATCCATACAGATAA |
| | | | | ATTCTAATAAAAACTGGGAAGAAGGTATAGAGTTCTTCTATAATA |
| | | | | AATTACACAATGATTCCAGTATTTCTAGTCAAGATAAAGATGATCT |
| | | | | GTTAATTGAAGCAGCTTTATCTGCAGTAAAGGGTTACAAAGAAGT |
| | | | | AGACACCATAGAGTTTTGCCTGTCTAAAATGGATGATGAGCAAAA |
| | | | | GAAAAAATTACTAGATAGAGATTATAAGGAAAATACTTATTATGC |
| | | | | AGTGTTGAATGTGCTAGTAGGTCAGTATTACTTTGATTCTTTTATG |
| | | | | GAATTAAGCCGATTGTGTAGTCAGATTGAATGTGAACGTTACACA |
| | | | | ACTTTTTTATCTTCATTATCAGATCAAGTACTGAAGAATCCAGATC |
| | | | | TGTCTGAAGAAACAAAAAAATGTATGATGAATGTTTGGGAACGTA |
| | | | | TAATAAAATTAAAAACTCAAGACCGCGGGGAGCAATCTATTTCCT |
| | | | | CTATTTTTGTAGACTATTCAGTTACATATACAATAGCAAATTTAAT |
| | | | | TGTGGATCCAAGTAGACAAGGGGTAAGTAAAGAAGAAATATTAG |
| | | | | GGAAGATATTAAAGCACGTAAAAGAAATGAGTGGTGAAGAGATG |
| | | | | ATAAAGGTTAAAGATTCTGTATTAAGTAAAATTCAGTTATTTCATG |
| | | | | GGGGTAAAAAATTGCAGTTAGGAGAACAAGTATTTTCTAAATTAG |
| | | | | CTCAAGAAGCTTCTAAAGAATCAATTTTGCGTGAAGCTGGTGATA |
| | | | | CTTTGCCACAGTCAAGTCTCAGTACGACTGATACCCCATATAATAT |
| | | | | AAAATCTTTAAGCCATAGCAAATAG (SEQ ID NO: 17) |
| CinA | wPa_0294 | amino | | MESGLDHNYNKILDILKGAIKGDDNQVKARKHLRVERWLRAYIQUE |
| wPip | | acid | | DFDEEKLIFFSDIFSDNSCWDGIKLKNKAVGERLTEEKNKNGKENPLD |
| | | | | LADRYYLACKYCLEDKIPGLFEQVFMRFKRSAFEEDGSDDDLRRELL |
| | | | | ENTEETSPIEAFWSFLIDKQIGKLNEYKSVEGLQKSIQINSNKNWEEGIE |
| | | | | FFYNKLHNDSSISSQDKDDLLLEAALSAVKGYKEVDTIEFCLSKMDDE |
| | | | | QKKKLLDRDYKENTYYAVLNVLVGQYYFDSFMELSRLCSQIECERYT |
| | | | | TFLSSLSDQVLKNPDLSEETKKCMMNVWERIIKLKTQDRGEQSISSIFV |
| | | | | DYSVTYTIANLIVDPSRQGVSKEEILGKILKHVKEMSGEEMIKVKDSV |
| | | | | LSKIQLFHGGKKLQLGEQVFSKLAQEASKESILREAGDTLPQSSLSTTD |
| | | | | TPYNIKSLSHSK (SEQ ID NO: 18) |
| CinB | wPa_0295 | nucleotide | | ATGCCAAGTAATGTCAAGCCGCTTGAGTTGGTACAGCTTCTGTTAA |
| wPip | | | | TGAGAAATAAATCAAAAGACGAGTTCCTAGATTTTCAAAAAAGGT |
| | | | | TCCAATCGTTTATCAATCAATCTCCTTCTTTTTTGCATTCAGTTGGA |
| | | | | AAGCCAGGCTTTTTCCCTAGTTTCTTTTTTGGTATGTTTGCTACTGT |
| | | | | ATTAGACACAGAACTTGCTACTAAAATTGGTATTAAAAAACTTCAT |
| | | | | TTTCGTTTTGATGATAATAGAACTTTAAAAATAGCTATATTAACTA |
| | | | | ATGAGGGACTTAAGTGTATAACGATGTCTGATCAAGTTGATGGTA |
| | | | | ACATGCATCTAAAGTTCTCTCAAGGAGAGTTAGAAAAAATAGCAC |
| | | | | AGAAATGGAAAATGGGAGCAGAGTTTGATAAACTAGAAAAAGAA |
| | | | | GAGCATGAAATAACAATTACAGGAAAAGAAGTAAAGCACGGAAA |
| | | | | GGTTGATCCAGCTTTTAGTAAAAAGACTGATTATTCACAAAAAGG |
| | | | | TTTTACAGAAATAGAAAAAGATCGTGACCAACAAGACCTAGAGAG |
| | | | | CTTAATTTCAAAATTGAGTAATCAAGATTTCGAAGAAGTAAAAAA |
| | | | | GAACGCTAGAAGAATGTTTAATTATATTACAAATGTCTATAAGAA |
| | | | | ATATGAAAAAGAAACTCTATTTAGCGGTAAAGAATCAAGTCATCA |
| | | | | TGGGTTTTTAGCTGGGTTTTTGATAAATTTTAAGTATCGTTTTCACC |
| | | | | TAAAACTTTATCTCGAATTATTTGCTGGAAAAGGTTACGCAGACAT |
| | | | | TATTTTGCTTGTGCGCGGTTCTGATAAGTCGCTAAGCTCTATTCCTA |
| | | | | TTATTATTGAGCTTAAAGCAGGTACTGGTGAGATAAGTACAGTGA |
| | | | | TAAAAGCATTGAAGCAAGCACAAGATTATGTTAAGGGCTCTTTTTC |
| | | | | TAACTCTATAAGAATGATTACTATAGCTAATGAAGCTATTTGTGTA |
| | | | | GGATTAAATTTTGACATGGTTCATCACGAAAATGTTAAAATTGATG |
| | | | | TAGAAAATTTTCTTAGTCGAGAAGGTAATTCTGTAATAGAAAAGTT |
| | | | | ACTTGGCACTGAAGCAACGAATGCTGAGGTGATAAGAACACAGCT |
| | | | | AGAGTATCTTTACTATGGAATTGTTTGGAGCAATGGTGGAAGTGAT |
| | | | | AATATTAATTATGTCAGCAGAATGATCTTAGGTCAGCTAGTACTTA |
| | | | | TTTCTAATATTATTAAGCGTGAAAAGTTAGGTAAACATATTTTTAT |
| | | | | TTATGATCAAAATGATAAAATGGTTACTGGATCACAGAAACGCCC |
| | | | | AGAAGCAGCAAAAGAAAGTATTGAGGATTGTGTTACAACTATAGT |
| | | | | GCTAACTTTAGGTAAGAAGGTGCTTATACTCAACATAAATGAAAA |
| | | | | AAATGAATTTGCATTGAGAGTGCCAGATAATAAAGGAATTCCTAT |
| | | | | TGAAAATATTAGGAGAATTCAAAACGTCAATGACATAAAGATACA |
| | | | | AGAAATAACCTGTAACTTATACAGTACGCCTAGTAATAAGAATCC |
| | | | | ATTTGATCAGTACTGTAATAAGAATAAGGGAATTACAGTAAATAC |
| | | | | GTATGACTCATTGGACAAATACAAAAGAGGTAAAGAAATTTTACA |
| | | | | AGGTAATTTTACTCGAATTGTGGAAAATAAAAAATTTAAAGCAGC |
| | | | | TTTGAGCAAAGCTATAGAATCTGGTAAATATGATGATTACAAAAA |
| | | | | ACTATTTGAAGAAATTTCTCATATACTACATCCTTTCAAATCATTA |
| | | | | ATAAGCAATGAGGCTACATTTCAAGCTGTATTGCATGGTTTATTTA |
| | | | | GTAGCTACGGAGAAGATAATATAAAAGTTATTACTGAATTTCAAA |
| | | | | TAGGTGGTGGAGAGAAGTTGGATGTTATGTTGGTTATAAATGCTA |
| | | | | CTGATCAAAAAAAAGAATACCCCCCAGTTGGAATAGAGCTAAAAT |
| | | | | TTGCTAAGAAAGGAGAATTGGATAAAAAAGAAAAAGATGCTAAG |
| | | | | GACCAGTTGAAAAGATATAAAGAAGGTGAAGCGTATAAGGTAATT |
| | | | | ACTGATGCTGGCAAAGTGAAACTGATATATGCTGTTTTTAATAAAG |
| | | | | GTGCAACAGATGAAGGTTCCCTTATAAAAATTGGTAATGAGTTTGT |
| | | | | AGAGGTAGATGTAAGACATAGCTCTGTGGTTGCTTTTGGTCAACA |
| | | | | GCCAGGTAGTCTCCAACAACCTTATGTTAAACAAGCAGGTCTATCT |
| | | | | CGAGCAGTTAATCAGTGA (SEQ ID NO: 19) |
| CinB | wPa_0295 | amino | | MPSNVKPLELVQLLLMRNKSKDEFLDFQKRFQSFINQSPSFLHSVGKP |
| wPip | | acid | | GFFPSFFFGMFATVLDTELATKIGIKKLHFRFDDNRTLKIAILTNEGLK |
| | | | | CITMSDQVDGNMHLKFSQGELEKIAQKWKMGAEFDKLEKEEHEITIT |
| | | | | GKEVKHGKVDPAFSKKTDYSQKGFTEIEKDRDQQDLESLISKLSNQD |
| | | | | FEEVKKNARRMFNYITNVYKKYEKETLFSGKESSHHGFLAGFLINFK |
| | | | | YRFHLKLYLELFAGKGYADIILLVRGSDKSLSSIPIIIELKAGTGEISTVI |
| | | | | KALKQAQDYVKGSFSNSIRMITIANEAICVGLNFDMVHHENVKIDVE |
| | | | | NFLSREGNSVIEKLLGTEATNAEVIRTQLEYLYYGIVWSNGGSDNINY |
| | | | | VSRMILGQLVLISNIIKREKLGKHIFIYDQNDKMVTGSQKRPEAAKESI |
| | | | | EDCVTTIVLTLGKKVLILNINEKNEFALRVPDNKGIPIENIRRIQNVNDI |
| | | | | KIQEITCNLYSTPSNKNPFDQYCNKNKGITVNTYDSLDKYKRGKEILQ |
| | | | | GNFTRIVENKKFKAALSKAIESGKYDDYKKLFEEISHILHPFKSLISNE |
| | | | | ATFQAVLHGLFSSYGEDNIKVITEFQIGGGEKLDVMLVINATDQKKEY |
| | | | | PPVGIELKFAKKGELDKKEKDAKDQLKRYKEGEAYKVITDAGKVKLI |
| | | | | YAVFNKGATDEGSLIKIGNEFVEVDVRHSSVVAFGQQPGSLQQPYVK |
| | | | | QAGLSRAVNQ (SEQ ID NO: 20) |

1. A genetically modified arthropod, said arthropod comprising:
(i) at least one bacterial gene encoding a cytoplasmic incompatibility factor or a variant thereof;
(ii) a promoter operably linked to the at least one bacterial gene, wherein the promoter comprises a Gal4 binding site; and
(iii) a nos-Gal4:VP16 gene driver;
wherein the expression of the cytoplasmic incompatibility factor in a male arthropod causes a reduction in viable offspring in comparison to a male arthropod lacking the cytoplasmic incompatibility factor.

2. The arthropod of claim 1, further comprising an additional gene driver.

3. The arthropod of claim 2, wherein the additional gene driver is a nos-GAL4-tubulin gene driver.

4. The arthropod of claim 2, wherein the additional gene driver is an otu-Gal4:VP16 gene driver.

5. The arthropod of claim 1, further comprising a nos-GAL4-tubulin gene driver and an otu-Gal4:VP16 gene driver.

6. The arthropod of claim 1, wherein the at least one bacterial gene is from Wolbachia.

7. The arthropod of claim 1, wherein the at least one bacterial gene is from wMel.

8. The arthropod of claim 1, wherein the at least one bacterial gene encodes the cytoplasmic incompatibility factor CifA (WD0631).

9. The arthropod of claim 1, wherein the at least one bacterial gene encodes the cytoplasmic incompatibility factor CifB (WD0632).

10. (canceled)

11. The arthropod of claim 1, wherein the at least one bacterial gene is from Wolbachia pipientis.

12. The arthropod of claim 1, wherein the at least one bacterial gene encodes the cytoplasmic incompatibility factor CidA^{wPip} (wPa_0282).

13. The arthropod of claim 1, wherein the at least one bacterial gene encodes the cytoplasmic incompatibility factor CidB^{wPip} (wPa_0283).

14. (canceled)

15. The arthropod of claim 1, wherein the at least one bacterial gene encodes the cytoplasmic incompatibility factor CinA^{wPip} (wPa_0294).

16. The arthropod of claim 1, wherein the at least one bacterial gene encodes the cytoplasmic incompatibility factor CinB^{wPip} (wPa_0295).

17. (canceled)

18. The arthropod of claim 1, wherein the reduction in viable offspring is greater than 50%.

19. The arthropod of claim 1, wherein the arthropod is an insect.

20. The arthropod of claim 19, wherein the insect is selected from the genera consisting of Aedes, Culex and Anopheles.

21. (canceled)

22. The arthropod of claim 19, wherein the insect is Drosophila suzukii.

23. A method for controlling a population target arthropods, comprising:
providing at least one bacterial gene encoding a cytoplasmic incompatibility factor or a variant thereof, and a promoter operably linked to the at least one bacterial gene, wherein the promoter comprises a Gal4 binding site;
(ii) transforming a population of male arthropods with the at least one bacterial gene, wherein the male arthropods comprise a nos-Gal4:VP16 gene driver; and
(iii) releasing the male arthropods amongst a population of target arthropods, wherein the release of the male arthropods reduces the population of target arthropods.

24.-43.(canceled)

44. A method for controlling a population of target arthropods, comprising:
(i) providing a genetically modified bacterium comprising:
a. at least one bacterial gene encoding a cytoplasmic incompatibility factor or a variant thereof, and
b. a promoter operably linked to the at least one bacterial gene, wherein the promoter comprises a Gal4 binding site;
(ii) infecting a population of replacement arthropods with the genetically modified bacterium, wherein the replacement arthropods comprise a nos-Gal4:VP16 gene driver; and
(iii) releasing the replacement arthropods amongst a population of target arthropods, wherein the release of the replacement arthropods reduces the population of target arthropods.

45.-64. (canceled)
